(19) United States
(12) Patent Application Publication
(10) Pub. No.: US 2023/0357757 A1
(43) Pub. Date: Nov. 9, 2023

(54) SELF-PURIFIED NUCLEIC ACID ENCODED LIBRARIES

(71) Applicant: ETH ZURICH, Zurich (CH)

(72) Inventors: Dario Neri, Zurich (CH); Jörg Scheuermann, Zurich (CH); Michelle Keller, Zurich (CH); Dimitar Petrov, Zurich (CH); Yuichi Onda, Osaka (JP); Gabriele Bassi, Otelfingen (CH)

(21) Appl. No.: 18/032,513

(22) PCT Filed: Oct. 21, 2021
(86) PCT No.: PCT/EP2021/079294

§ 371 Date: Apr. 18, 2023

(30) Foreign Application Priority Data
Oct. 23, 2020 (EP) 20203475.7

(51) Int. Cl. C12N 15/10

(52) U.S. Cl. CPC C12N 15/1068

(57) ABSTRACT

This invention relates to methods for producing nucleic acid encoded compounds and libraries of nucleic acid encoded compounds. A nascent compound that comprises a scaffold connected to a solid support by a linker is covalently attached to one or more chemical building blocks to form a chemical portion attached to the scaffold. Coding oligonucleotides encoding the one or more chemical building blocks are covalently attached to the nascent compound to form a coding nucleic acid portion attached to the scaffold. A cleaving group is attached to the chemical portion, nucleic acid portion, or scaffold of the compound. The linker is then reacted with the cleaving group, such that the linker is cleaved and the compound released from the solid support. Nucleic acid encoded compounds and libraries and methods for their production are provided.

### FIELD

The present invention relates to nucleic acid encoded chemical libraries, particularly self-purified nucleic acid encoded chemical libraries, and methods for production and application thereof.

### BACKGROUND

DNA encoded chemical libraries (DEL) are powerful tools for drug discovery. The first methods proposed for the production of DNA-encoded chemical libraries employed alternating stepwise synthesis of a polymer (e.g. a peptide) and an oligonucleotide sequence (serving as a coding sequence) on a common linker (e.g. a bead) in split and pool cycles (Brenner, S. and Lerner, R. A. PNAS USA 89 (1992), 5381-5383; U.S. Pat. No. 5,573,905; WO93/20242). After affinity capture on a target protein, the population of identifier oligonucleotides of the selected library members would be amplified by PCR. The structures of the chemical entities would be decoded by sequencing the PCR products. It was postulated that encoding procedures could be implemented by a variety of methods, including chemical synthesis, DNA polymerization or ligation of DNA fragments (Brenner, S. and Lerner, R. A. PNAS USA 89 (1992), 5381-5383; U.S. Pat. No. 5,573,905; WO93/20242). Various methods of generating DNA-encoded chemical libraries have subsequently been described in the art (see for example Mannocci, L. et al. PNAS USA 105(46):17670-17675; Brenner, S. and Lerner, R. A. supra; Nielsen, J., et al., J. Am. Chem. Soc. 115 (1993); Needels et al., M. C., PNAS USA 90 (1993), 10700-10704; Gartner, Z. J., et al., Science 305 (2004), 1601-1605; Melkko, S., et al., Nat. Biotechnol. 22 568-574 (2004); Sprinz, K. I., et al., Bioorg. Med. Chem. Lett. 15 (2005), pp. 3908-3911; Leimbacher et al Chemistry. 2012 Jun. 18; 18(25):7729-37; Clark et al Nat Chem Biol. 2009 September; 5(9):647-54; WO2009/077173; WO2003/076943; EP3284851; EP3184674).

The established use of DEL technology enables the screening of a large number of compounds (typically in the order of 1 to 100 million) which are individually encoded by a specific nucleic acid tag and affinity-based screening of the entire DEL for a protein of interest can be performed in a single experiment. DEL technology is now widely used in the pharmaceutical industry.

To date, the variable yields of the individual synthesis steps in the construction of DELs have restricted the number of consecutive synthesis steps and the nature of building blocks which can be incorporated. Methods that allow the construction of DELs of increased size and/or purity would be useful.

### SUMMARY

The present inventors have developed a method of producing nucleic acid encoded libraries which self-purifies intact or complete library members from intermediates through selective cleavage from a solid support. This may, for example, facilitate the production of large and/or pure nucleic acid encoded libraries and/or nucleic acid encoded libraries in which the individual members have a complex structure and/or a large number of building blocks. Libraries produced by these methods may for example display improved screening performance and/or contain members capable of binding to large surfaces on target proteins.

A first aspect of the invention provides a method for producing a nucleic acid encoded compound which includes the steps of;
providing a nascent compound that comprises a scaffold connected to a solid support by a linker,
covalently attaching one or more chemical building blocks to the nascent compound to form a chemical portion attached to the scaffold,
covalently attaching coding oligonucleotides encoding the one or more chemical building blocks to the nascent compound to form a coding nucleic acid portion attached to the scaffold,
attaching a cleaving group to the chemical portion, nucleic acid portion, or scaffold of the compound, and
reacting the linker and the cleaving group, such that the linker is cleaved and the compound released from the solid support.

A second aspect of the invention provides a method for producing a nucleic acid encoded chemical library comprising, for each library member, the steps of;
providing a nascent member that comprises a scaffold connected to a solid support by a linker,
covalently attaching one or more chemical building blocks to the nascent member to form the chemical portion attached to the scaffold,
covalently attaching coding oligonucleotides encoding the one or more chemical building blocks to the nascent member to form a coding nucleic acid portion attached to the scaffold,
attaching a cleaving group to the chemical portion, nucleic acid portion, or scaffold,
reacting the linker and the cleaving group, such that the linker is cleaved and the member is released from the solid support.

In some embodiments of the first and second aspects, the cleaving group may be attached to the chemical portion (FIG. 1A). The selective release from the solid support may be initiated by the presence of a complete chemical portion, or a complete segment of the chemical portion.

In other embodiments of the first and second aspects, the cleaving group may be attached to the coding nucleic acid portion (FIG. 1B). The selective release from the solid support may be initiated by the presence of a complete nucleic acid portion, or a complete segment of the nucleic acid portion.

In other embodiments of the first and second aspects, the cleaving group may be attached to the scaffold (FIG. 1C). The selective release from the solid support may be initiated by the presence of a complete scaffold.

In some embodiments of the first and second aspects, the scaffold of the nascent compound or member may be additionally connected to the solid support by a second linker, such that the scaffold is connected to the solid support by a first linker and a second linker. The method may comprise;
providing a nascent compound or member that comprises a scaffold connected to a solid support by a first linker and a second linker,
covalently attaching one or more chemical building blocks to the nascent compound or member to form the chemical portion attached to the scaffold,
covalently attaching coding oligonucleotides encoding the one or more chemical building blocks to the nascent compound or member to form a coding nucleic acid portion attached to the scaffold,
attaching a first cleaving group to one of the coding nucleic acid portion chemical portion or scaffold,
attaching a second cleaving group to another of the coding nucleic acid portion, chemical portion or scaffold,
reacting the first linker and the first cleaving group, such that the first linker is cleaved, and
reacting the second linker and the second cleaving group, such that the second linker is cleaved and the compound or member is released from the solid support.

The first and second linkers may be cleaved sequentially in any order or simultaneously.

The compound or member is released from the solid support if it comprises (i) both a complete chemical portion or a complete segment of the chemical portion and a complete nucleic acid portion, or a complete segment of the nucleic acid portion (ii) both a complete chemical portion or a complete segment of the chemical portion and a complete scaffold, or (iii) both a complete coding nucleic acid portion, or a complete segment of the coding nucleic acid portion and a complete scaffold.

In some embodiments, the cleaving group or first cleaving group may be covalently attached to the chemical portion or the scaffold. Preferably, the cleaving group is covalently attached to the terminal chemical building block of the chemical portion.

In other embodiments, the cleaving group may be non-covalently attached to the chemical portion or scaffold. For example, by attaching an anchor oligonucleotide to the chemical portion or the scaffold, preferably to the terminal chemical building block of the chemical portion, and hybridizing said anchor oligonucleotide with an auxiliary oligonucleotide which is linked to a cleaving group. For example, a method of producing a nucleic acid encoded compound or chemical library may comprise the steps of;
attaching an anchor oligonucleotide to the chemical portion or scaffold,
providing an auxiliary oligonucleotide attached to a cleaving group,
hybridizing the attachment oligonucleotide with the auxiliary oligonucleotide, and
reacting the linker and the cleaving group, such that the linker is cleaved.

In some embodiments, the cleaving group may be covalently attached to the nucleic acid portion. Preferably, the cleaving group is covalently attached to the end of the nucleic acid portion.

In other embodiments, the cleaving group may be non-covalently attached to the nucleic acid portion, for example, by hybridizing said coding nucleic acid portion with an auxiliary oligonucleotide which is linked to a cleaving group. Preferably, the auxiliary oligonucleotide is hybridized to the end of the coding nucleic acid portion. For example, a method of producing a nucleic acid encoded compound or chemical library may comprise the steps of;
providing an auxiliary oligonucleotide covalently attached to a cleaving group,
hybridizing the auxiliary oligonucleotide to the coding nucleic acid portion, and
reacting the linker and the cleaving group, such that the linker is cleaved.

The linker may be transformed or activated after attachment to the chemical portion, scaffold, or the coding nucleic acid portion and before reaction with the cleaving group.

In some embodiments, the cleaving group may not require further transformation after attachment to the chemical portion, scaffold, or the coding nucleic acid portion and before reaction with the linker. In other embodiments, the cleaving group may be activated after attachment to the chemical portion, scaffold, or the coding nucleic acid portion and before reaction with the linker.

In some preferred embodiments, a capping step may be performed after each chemical building block addition, and optionally each coding oligonucleotide addition. This prevents the cleaving group from attaching to incomplete nucleic acid or peptide nucleic acid encoded compounds.

In the aspects described herein, in addition to cleaving the linker and releasing the compound or member, the cleaving group may form a covalent bond that links the end of chemical portion to the scaffold to generate a macrocycle. For example, the reaction of the linker and the cleaving group may generate a cyclisation element or cleavage moiety that covalently links the chain of chemical building blocks to the scaffold, such that the chemical entity displayed by the member is macrocyclic.

A third aspect of the invention provides a method for producing a nucleic acid encoded compound which includes the steps of;
providing a nascent compound that comprises a scaffold connected to a solid support by a first linker,
covalently attaching one or more chemical building blocks to the nascent compound to form a chemical portion attached to the scaffold,
covalently attaching coding oligonucleotides encoding the one or more chemical building blocks to the nascent compound to form a coding nucleic acid portion attached to the scaffold,
connecting the chemical portion of the compound to the solid support with a second linker,
cleaving the first linker,
cleaving the second linker, such that the compound released from the solid support.

First and second linkers according to the third aspect may be orthogonally cleavable.

In methods of the third aspect, compounds with complete chemical portions are covalently connected to the solid support by the second linker. Compounds with incomplete chemical portions are not covalently connected to the solid support by the second linker. Compounds with incomplete chemical portions are thus selectively released from the solid support by cleavage of the first linker. Compounds with complete chemical portions remain attached to the solid support by the second linker. Cleavage of the second linker selectively releases these compounds from the solid support.

Preferably, a capping step is performed after the addition of each chemical building block. Compounds with incomplete chemical portions may remain capped to prevent connection to the second linker.

In all of the first to third aspects described herein, the chemical building blocks may be added sequentially to the nascent member or compound to form a linear chemical portion (i.e. a chain of chemical building blocks) with an end attached to the nascent member and a free end. The coding oligonucleotides encoding each chemical building block may be added sequentially to the nascent member to form a linear coding nucleic acid portion. Methods of the invention may comprise covalently attaching a chemical building block to the nascent compound or member and covalently attaching a coding oligonucleotide encoding the chemical building block to the nascent member. This may be repeated one or more times to produce the chemical portion and the coding nucleic acid portion. Following the attachment of a chemical building block, any unreacted species which lack the attached chemical building block may be capped before addition of the next chemical building block.

A fourth aspect provides a nucleic acid encoded library produced by a method of the first to the third aspects.

These and other aspects and embodiments of the invention are described in more detail below.

### BRIEF DESCRIPTION OF THE FIGURES

FIG. 1 shows a schematic representations of possible solid support compounds. The solid support compounds in (A), (B) and (C) each comprise a solid support attached to the scaffold by a linker. In each of (A), (B), and (C), the chemical portion, as well as the nucleic acid portion, is attached to the scaffold. In (A), the cleaving group is attached to the chemical portion. In (B), the cleaving group is attached to the nucleic acid portion, In (C), the cleaving group is attached to the scaffold. Abbreviations: Cleaving group (CG).

FIG. 2 shows a schematic representation of the self-purification of a nucleic acid or peptide nucleic acid encoded compound or chemical library member. In the embodiment shown, the cleaving group is attached to the chemical portion. The reaction of the cleaving group with the chemical portion releases the self-purified compound from the solid support. In this embodiment shown, the reaction of the cleaving group with the linker results in a product (CG/Linker Product; or cleavage moiety) which connects the chemical portion to the scaffold. A portion of the cleaved linker may remain attached to the solid support compound. Abbreviations: Cleaving group (CG).

FIG. 3 shows a schematic representation of the self-purification of a nucleic acid or peptide nucleic acid encoded compound or chemical library member. In the embodiment shown, the cleaving group is attached to the chemical portion. The reaction of the cleaving group with the chemical portion releases the self-purified compound from the solid support. In this embodiment shown, the reaction of the cleaving group with the linker results in a cleaving group product (CG product) which is attached to the chemical portion. A portion of the cleaved linker may remain attached to the solid support compound. Abbreviations: Cleaving group (CG).

FIG. 4 shows a schematic representation of the self-purification of a nucleic acid or peptide nucleic acid encoded compound or chemical library member, wherein two orthogonal linkers for separate chemical portion and coding nucleic acid portion purification are used. Two orthogonal cleaving groups are used, wherein cleaving group 1 (CG1) is attached to the chemical portion and the cleaving group 2 (CG2) is attached to the nucleic acid portion. In the embodiment shown, the cleaving group 1 (CG1) reacts with linker 1 to cleave linker 1 and result in a product (CG1/Linker 1 Product) which connects the chemical portion to the scaffold. In the embodiment shown, cleaving group 2 (CG2) reacts with linker 2 to cleave linker 2 and result in a cleaving group product (CG2 product) which is attached to the nucleic acid portion. Upon cleavage of both linkers, the self-purified compound is released from the solid support. Portions of each of the cleaved linkers may remain attached to the solid support compound. Abbreviations: Cleaving group (CG), cleaving group 1 (CG1), cleaving group 2 (CG2).

FIG. 5 shows a schematic representation of two possible arrangements of chemical building blocks in a solid support compound. In (A), a linear arrangement of chemical building blocks is shown. In (A), building block 1 (chemical building block1) is attached to the scaffold. In (A) building block 2 (chemical building block2) is attached to building block 1 (chemical building block1). In (A), building blocks are arranged to form a linear chain of building blocks, ending with the terminal building block (chemical building blockn), which is then attached to the cleaving group (CG) in the embodiment shown. In (B), a crosslinked structure of the chemical portion is showed, which is formed by first attaching three building blocks to form a linear chain, and then attaching building block 4 (chemical building block4) both to building block 3 (chemical building block3) and to building block 1 (chemical building block1). In the embodiment shown in (B), further building blocks may be attached to building block 3 (chemical building block3), ending with the terminal building block (chemical building blockn), which is in turn attached to the cleaving group (CG). Abbreviations: Cleaving group (CG), building block (chemical building block), building block 1 (chemical building block1), building block 2 (chemical building block2), building block 3 (chemical building block3), building block 4 (chemical building block4), terminal building block (chemical building blockn).

FIG. 6 shows a schematic representation of two possible structures of self-purified compounds. The self-purified compounds each comprise a scaffold attached to a nucleic acid portion, as well as a chemical portion. In embodiment (A), a self-purified compound of linear structure is shown. In (A), building blocks are arranged in a linear fashion, and the self-purification reaction results in a cleaving group product (CG Product) attached to the terminal building block (chemical building blockn). In (A), the scaffold, building blocks, and cleaving group product (CG Product) are arranged in a linear fashion. In embodiment (B), a self-purified compound of cyclic structure is shown. In (A), building blocks are arranged in a linear fashion, and the self-purification reaction results in a product (CG/Linker Product) which connects the terminal building block (chemical building blockn) to the scaffold. In (B), the scaffold, building blocks, and cleaving group/linker product (CG/Linker Product) are arranged in a cyclic fashion.

FIG. 7 shows a schematic representation of two possible structures of self-purified compounds. The self-purified compounds each comprise a scaffold attached to a nucleic acid portion, as well as a chemical portion. In embodiment (A), a self-purified compound of branched structure is shown. In (A), building block 1 (chemical building block1), building block 2 (chemical building block2), building block 4 (chemical building block4) and the terminal building block (chemical building blockn) are arranged in a linear structure. In (A), building block 3 (chemical building block3) is attached to building block 2 (chemical building block2) as an appendage. In (A), building blocks are arranged in a branched fashion, and the self-purification reaction results in a cleaving group product (CG Product) attached to the terminal building block (chemical building blockn). In (A), the scaffold, building blocks, and cleaving group product (CG Product) are arranged in a branched fashion. In embodiment (B), a self-purified compound of bicyclic structure is shown. In (B), building blocks 1 to the terminal building block are arranged in a linear fashion, and additionally, building block 2 (chemical building block2) and building block 4 (chemical building block4) are cross-linked. In (B) the self-purification reaction results in a product (CG/Linker Product) which connects the terminal building block (chemical building blockn) to the scaffold. In (B), the scaffold, building blocks, and cleaving group/linker product (CG/Linker Product) are arranged in a bicyclic fashion. Abbreviations: Cleaving group (CG), building block (chemical building block), building block 1 (chemical building block1), building block 2 (chemical building block2), building block 3 (chemical building block3), building block 4 (chemical building block4), terminal building block (chemical building blockn).

FIG. 8 shows a schematic representation of selective cleavage of pure nucleic acid or peptide nucleic acid encoded compounds or chemical library members from solid support. Solid support compounds which comprise a completely synthesised structure between the linker and the cleaving group (CG), are released from the solid support (top panel). Truncated products, which are capped in the synthesis, are not cleaved from the solid support as they do not comprise a cleaving group (CG) (bottom panel). Abbreviations: Cleaving group (CG).

FIG. 9 shows a schematic representation of selective ligation of sequential nucleic acid codes. Within the nucleic acid portion, code 1 can only be attached to code 2, and not to code 3. Compounds which comprise complete code 1 ligated to code 2 may be attached to code 3 (top panel). In contrast, compounds which are truncated due to unsuccessful attachment of code 2 to code 1, may not be ligated to code 3 in a subsequent ligation (bottom panel).

FIG. 10 shows a schematic representation of the selective installation of Linker 2 for a completely synthesized solid support compound. For complete solid support compounds, the chemical portion may be linked to the solid support, or a branching region between the solid support and Linker 1, through Linker 2 (top panel). In contrast, Linker 2 may not be successfully installed for a compound with an incomplete chemical portion, such as a capped compound (bottom panel).

FIG. 11 shows a schematic representation of the selective release of nucleic acid or peptide nucleic acid encoded compounds or chemical library members with incomplete chemical portions from the solid support. The cleavage of linker 1 releases compounds without a second linker (Linker 2) from the solid support. Nucleic acid encoded compounds with truncated chemical portions are released from the solid support by the cleavage of Linker 1 (bottom panel). Nucleic acid encoded compounds or library members comprising both Linker 1 and Linker 2 remain attached to the solid support after the cleavage of Linker 1 (top panel). The selective release of compounds with truncated or capped chemical portions provides a self-purifying effect.

FIG. 12 shows a schematic representation of the release of nucleic acid or peptide nucleic acid encoded compounds or chemical library members with complete chemical portions from the solid support by the cleavage of the second linker (Linker 2). Linker 2 follows the cleavage of Linker 1 and washing of the solid support to remove any undesired impure compounds.

FIG. 13 shows analytical LCMS data for the preparation of a 5′-azido modified single-stranded oligonucleotide (Example 1). In (A), the chromatogram measuring 260 nm absorbance is shown for the starting material oligonucleotide (Sequence 1). The deconvoluted (decon.) mass spectrum is shown in (C) for the starting material oligonucleotide (Sequence 1). In (B), the chromatogram measuring 260 nm absorbance is shown for the product, 5′-azido modified single-stranded oligonucleotide. (D) shows the deconvoluted mass spectrum for the product peak.

FIG. 14 shows analytical LCMS data for the cleavage of a nascent library member (Example 2). (A), and (B) show chromatograms measuring 260 nm absorbance. (C), and (D) show the mass spectrum of the product peak at 3.84 min, and the deconvoluted mass spectrum, respectively.

FIG. 15 shows chemical structures detailing the activation of the MeDbz linker in a nucleic acid encoded library member (Example 4, Example 5). Transformation (A) shows the reaction of MeDbz with p-nitrophenyl chloroformate. Reaction (B) shows the final step in the linker activation. This is an example for the linker activation step for self-purification.

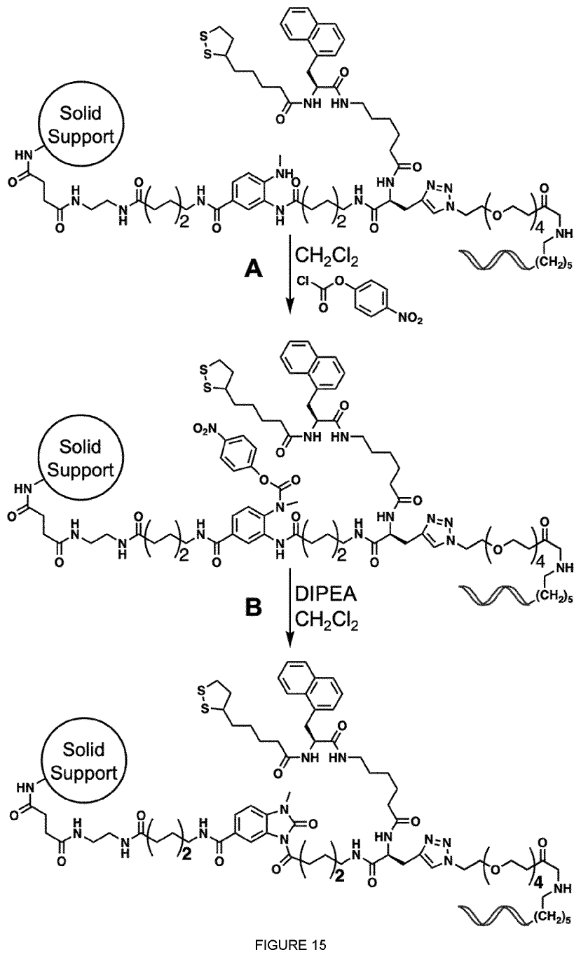

FIG. 16 shows chemical structures for the reduction of the disulfide bond in the lipoic acid cleaving group (Example 4, Example 5). This is an example for the cleaving group deprotection step for self-purification.

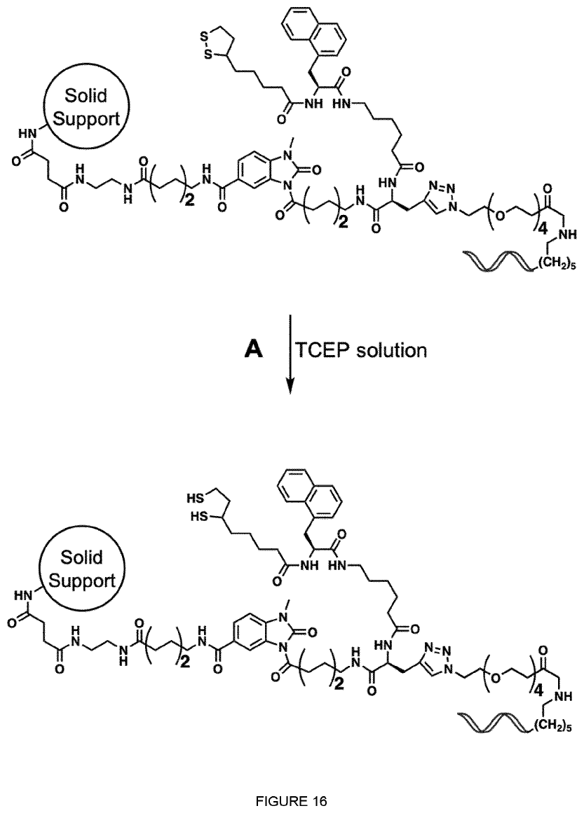

FIG. 17 shows the self-elution of a nucleic acid encoded library member (Example 4, Example 5). The chemical structures shown illustrate the reaction of the cleaving group comprising a thiol with the activated MeNbz linker. This releases the self-eluted library member from solid support. This step yields cyclic compounds with a thioester. Two possible cyclic compounds may be obtained, one with either thiol of the cleaving group having reacted with the linker.

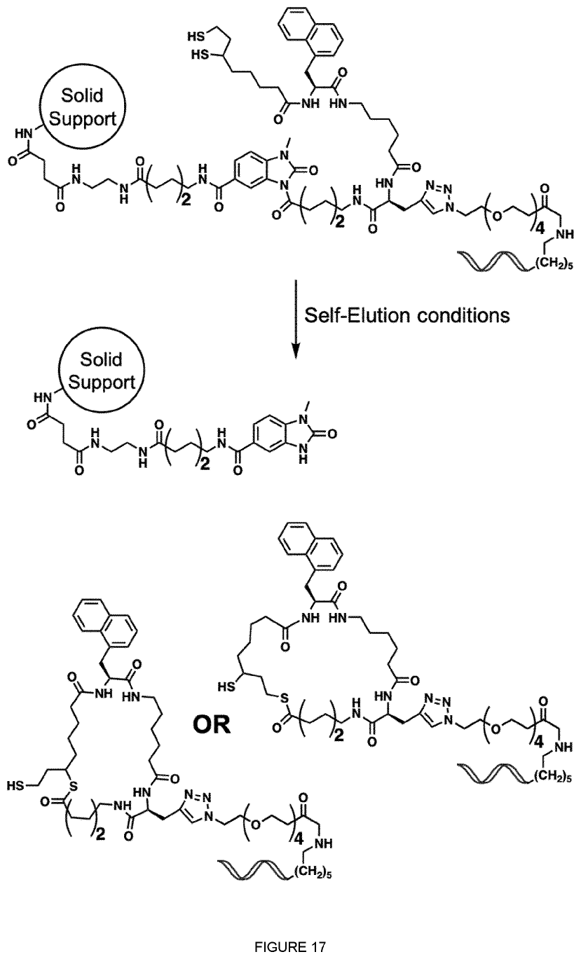

FIG. 18 shows chemical structures for the hydrolysis of cyclic self-purified library members with a thioester bond (Example 4). The product obtained after hydrolysis of the thioester is linear in this case.

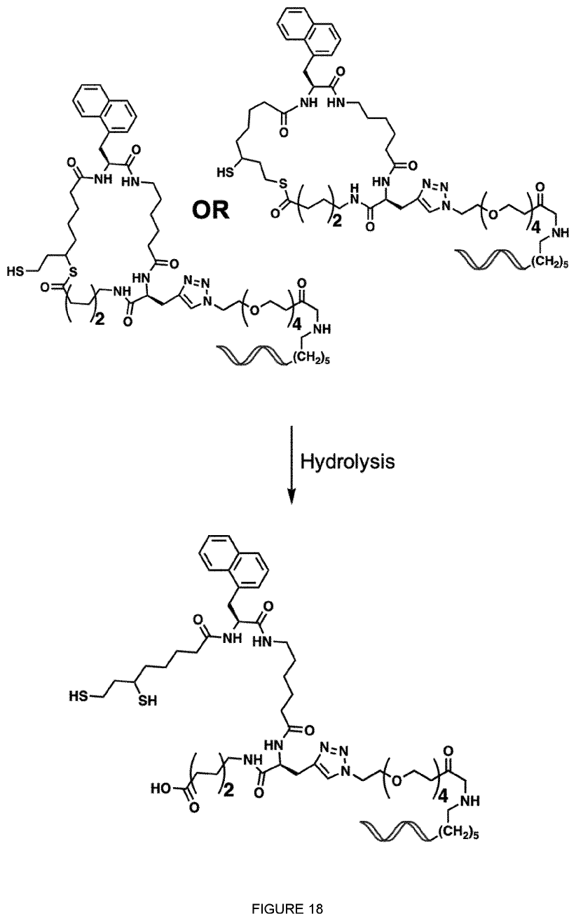

FIG. 19 shows chemical structures and analytical LCMS spectra for self-eluted model nucleic acid encoded library members observed after precipitation of the supernatant of an additional incubation step after TCEP reduction (Example 4). The chemical structures (A), and (B) were observed in the LCMS spectra in (C), and (D). (B) is the linear self-eluted product formed by the hydrolysis of the cyclic thioester intermediate. (A) is the linear self-eluted product formed by the ring opening of the cyclic thioester intermediate by ethanol, which is added for the precipitation step. (C) is the chromatogram for absorbance at 260 nm, and (D) is the chromatogram for absorbance at 280 nm.

FIG. 20 shows mass spectra obtained from LCMS analysis of a self-eluted model nucleic acid encoded library member (Example 4). (A) and (B) show the mass spectrum and the deconvoluted mass spectrum, respectively, for the self-eluted library member compound illustrated in FIG. 19A. (C) and (D) show the mass spectrum and the deconvoluted mass spectrum, respectively, for the self-eluted library member compound illustrated in FIG. 19B.

FIG. 21 shows chemical structures and LCMS spectra for the self-eluted nucleic acid library member prepared in Example 5. (A) shows the structures of the two possible cyclic self-eluted nucleic acid encoded library members. (B) shows the chromatogram at 260 nm, and (C) is the total ion current (TIC). The peak with the mass corresponding to the cyclic self-eluted library members is observed at 5.88 min.

FIG. 22 shows chemical structures and LCMS spectra for the self-eluted nucleic acid library member prepared in Example 5. (A) shows the structures of the two possible cyclic self-eluted nucleic acid encoded library members. (B), and (C) are the mass spectrum for the peak for the self-eluted nucleic acid library member, and the corresponding deconvoluted mass spectrum, respectively. The mass of the desired cyclic self-eluted nucleic acid encoded library member was observed.

FIG. 23 shows a reaction scheme for steps in the synthesis of a nascent library member (Example 6, Example 7). First, amine-functionalized solid support was coupled to Fmoc-Lys(Mtt)-OH (A). Then, Fmoc deprotection and coupling to HMBA follow (B). Thereafter, amide coupling to Fmoc-Pra-OH is shown (C).

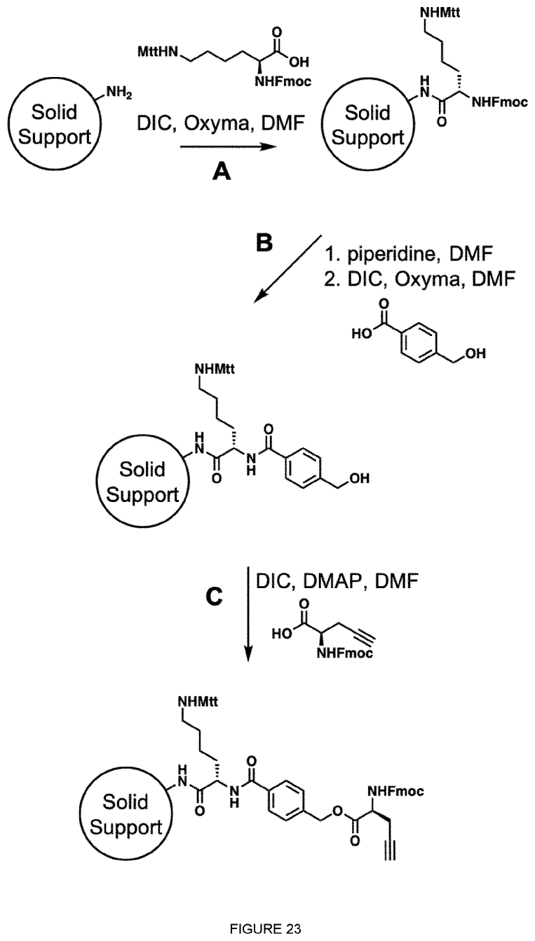

FIG. 24 shows a reaction scheme for steps in the synthesis of a nucleic acid encoded library member (Example 6). First, Fmoc deprotection of the product in FIG. 23 was followed by coupling to a 1-(1,1-Dimethylethyl) butanedioate building block (A). Then, the distal group of building block was tBu deprotected, and the lysine side chain was Mtt deprotected with TFA in dichloromethane (B).

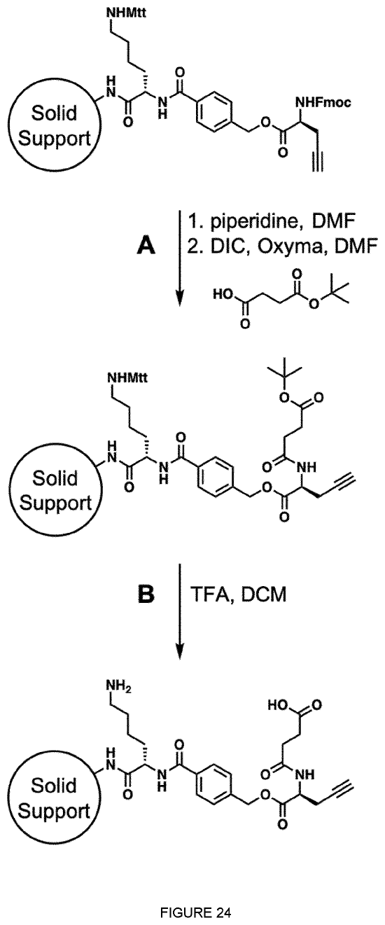

FIG. 25 shows a reaction scheme for steps in the synthesis of a nucleic acid encoded library member (Example 6). Oligonucleotide attachment by copper-catalyzed azide-alkyne cycloaddition (CuAAC) is shown in (A). (B) Shows an amide coupling reaction performed on solid support in the presence of DNA. (B) is the first step in the installation of the second linker.

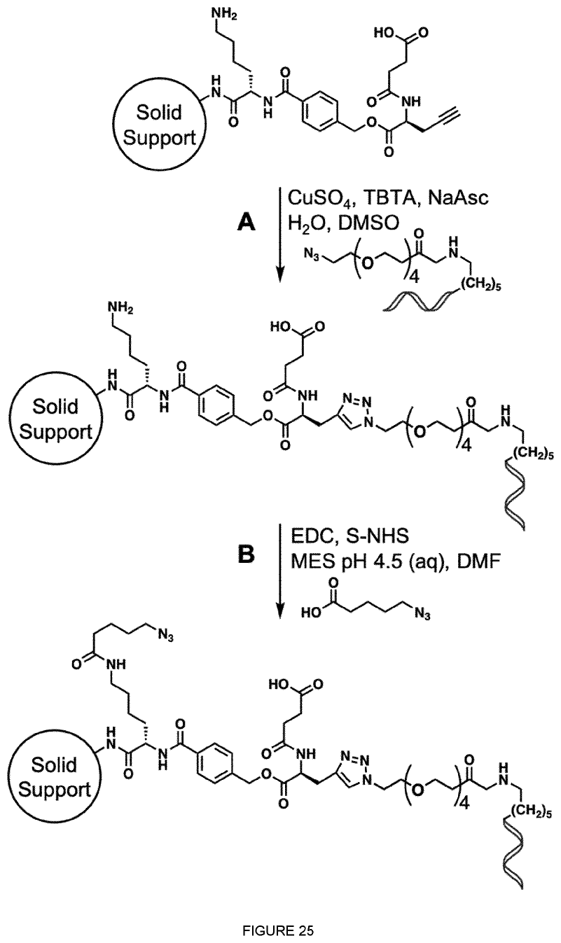

FIG. 26 shows a reaction scheme for steps in the synthesis of a nucleic acid encoded library member (Example 6). The product shown in FIG. 25 was reacted with a Dbz linker derivative with an alkyne by amide coupling (A). Then, a CuAAC reaction was performed in order to obtain a cyclic solid support compound (B). Cyclized compounds comprise a first linker, HMBA linker, and a second linker, a Dbz linker.

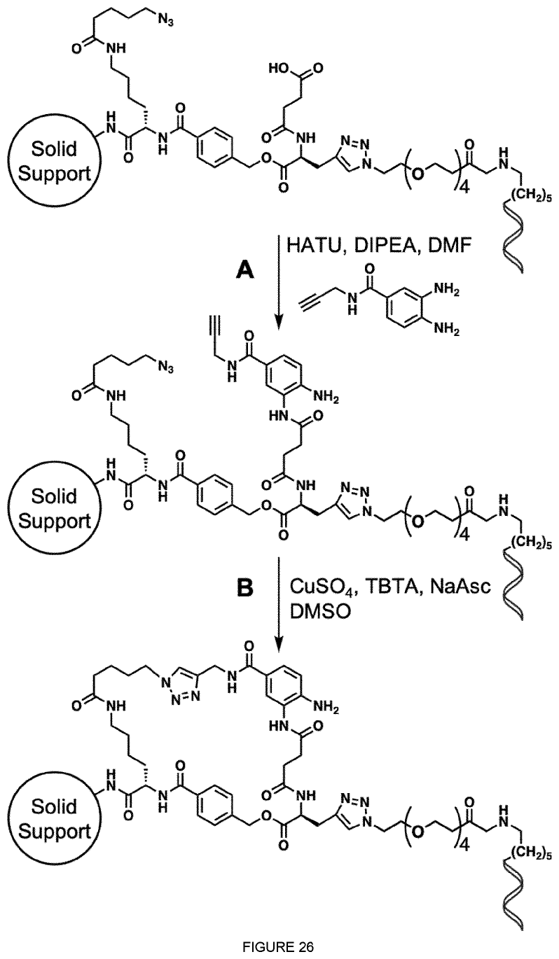

FIG. 27 shows a reaction scheme for the first step in the self-purification of a nucleic acid encoded library member (Example 6). The first linker of the product in FIG. 26 was cleaved in basic conditions. In this step, non-cyclized compounds were released from solid support. In this step, undesired side-products may be washed away from the solid support.

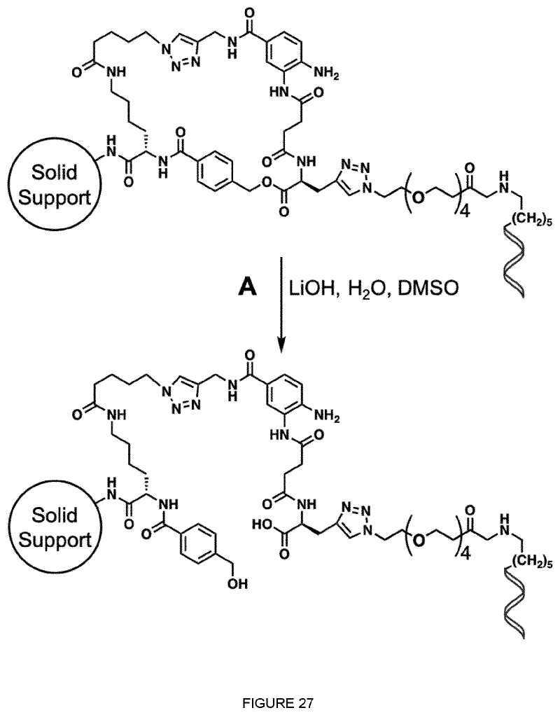

FIG. 28 shows a reaction scheme for the cleavage of the second linker for the self-purification of a nucleic acid encoded library member (Example 6). First, the second linker, Dbz, was activated using isopentyl nitrite (A). In (B), the activated linker was then cleaved in basic conditions in water and DMSO. This released the self-purified nucleic acid encoded library member from solid support.

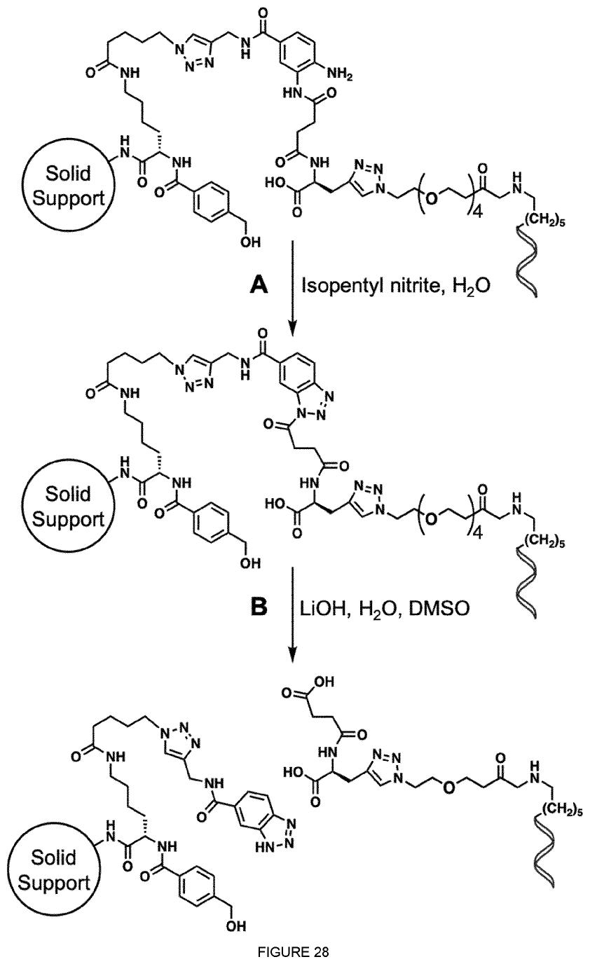

FIG. 29 shows LCMS spectra for samples prepared in Example 6. (A) shows an LCMS spectrum obtained by cleaving an intermediate in the synthesis of a nucleic acid encoded library member from solid support. The intermediate analyzed is after coupling to the Dbz linker derivative, and before the CuAAC. The cleavage of the HMBA linker yields the desired intermediate, shown at 4.01 min in chromatogram (A). The chemical structure of the desired intermediate is shown in (A). Additionally, the sample analyzed comprises undesired intermediate products. These may, for example, be compounds which did not undergo a previous amide coupling step in the synthesis. (B) shows a chromatogram obtained by cleaving the first linker, an HMBA linker, after the final CuAAC step in the synthesis of a nucleic acid encoded library member (Example 6). This is the first step in the self-purification of the nucleic acid encoded library member. In this step, uncyclized undesired products are released from solid support. Comparison of chromatogram (B) with chromatogram (A) shows that all compounds are cleaved from solid support in (B), except the desired intermediate in (A), since this compound comprises an alkyne and has undergone an CuAAC before the cleavage in (B). This illustrates that the CuAAC reaction has been successful and only undesired products are released from solid support in this step. (C) shows a chromatogram of the sample obtained after cleaving the second linker, Dbz. The chromatogram shows the self-purified nucleic acid encoded library member. The structure of the desired, self-purified nucleic acid encoded library member is shown in (C).

FIG. 30 shows LCMS spectra for the self-purified nucleic acid encoded library member prepared in Example 6. (A) shows the chromatogram for 260 nm absorbance. (B) shows the chromatogram for 280 nm absorbance. (C) shows the mass spectrum of the product peak at 3.94 min. (D) shows the deconvoluted mass spectrum of the product peak at 3.94 min. The mass corresponding to the desired self-purified nucleic acid encoded library member is observed.

FIG. 31 shows a reaction scheme for steps in the synthesis of a model nucleic acid encoded library member (Example 7). (A) shows Fmoc deprotection followed by amide coupling to Fmoc-Dbz-OH. (B) shows Mtt deprotection of the lysine side chain in the solid support compound.

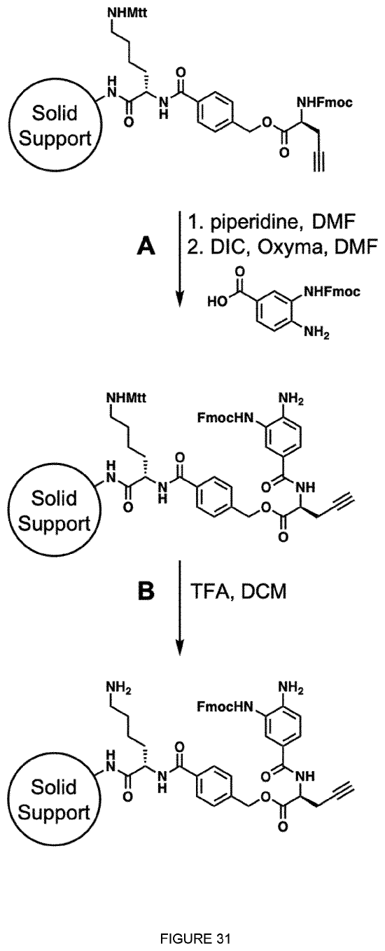

FIG. 32 shows a reaction scheme for steps in the synthesis of a model nucleic acid encoded library member (Example 7). Oligonucleotide attachment by copper-catalyzed azide-alkyne cycloaddition (CuAAC) is shown in (A). (B) Shows an amide coupling reaction performed on solid support in the presence of DNA. (B) is the first step in the installation of the second linker.

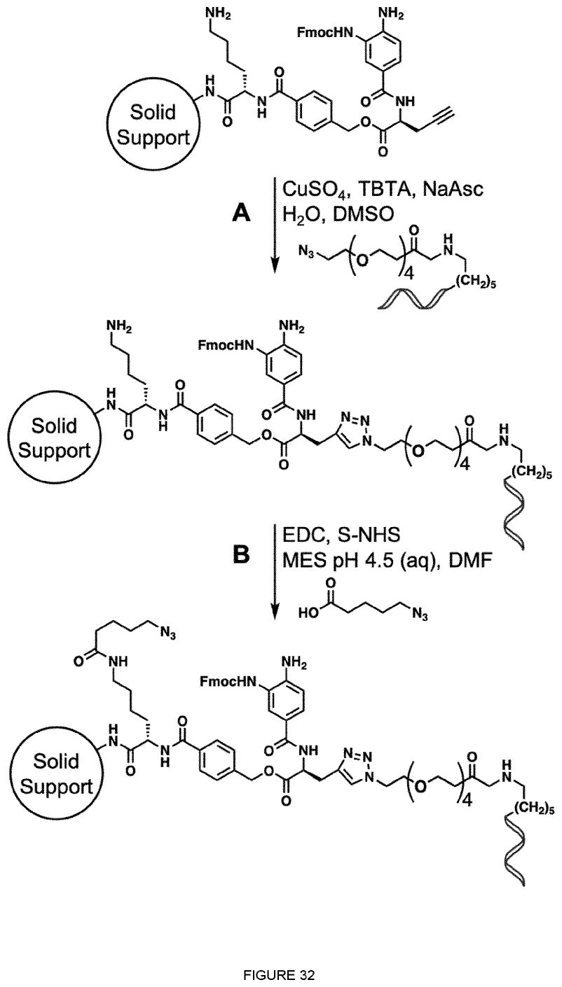

FIG. 33 shows a reaction scheme for steps in the synthesis of a model nucleic acid encoded library member (Example 7). Fmoc deprotection of the product shown in FIG. 32 was followed by amide coupling to hexynoic acid (A). Then, a CuAAC reaction was performed in order to obtain a cyclic solid support compound (B). Cyclized compounds comprise a first linker, HMBA linker, and a second linker, a Dbz linker.

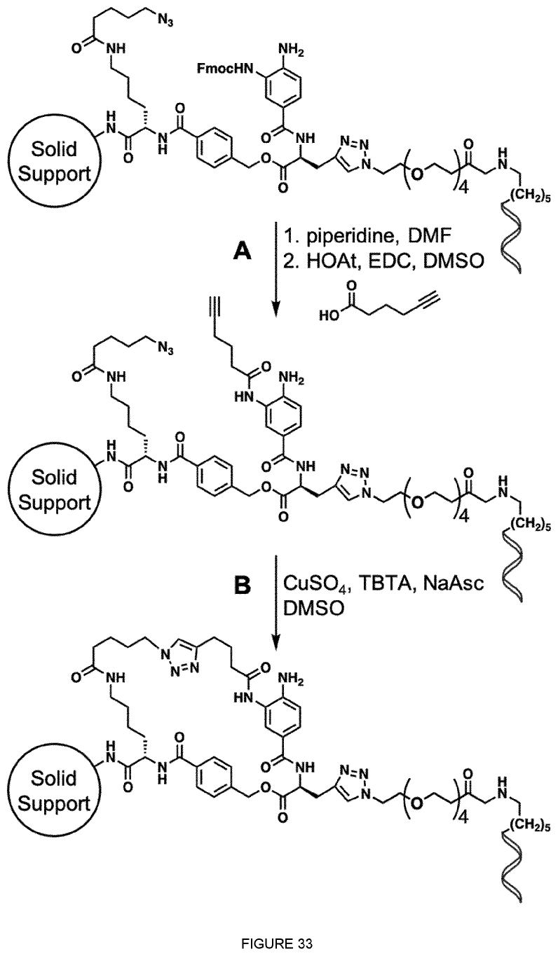

FIG. 34 shows a reaction scheme for the first step in the self-purification of a nucleic acid encoded library member (Example 7). The first linker of the product in FIG. 33 was cleaved in basic conditions. In this step, non-cyclized compounds were released from solid support. In this step, undesired side-products may be washed away from the solid support.

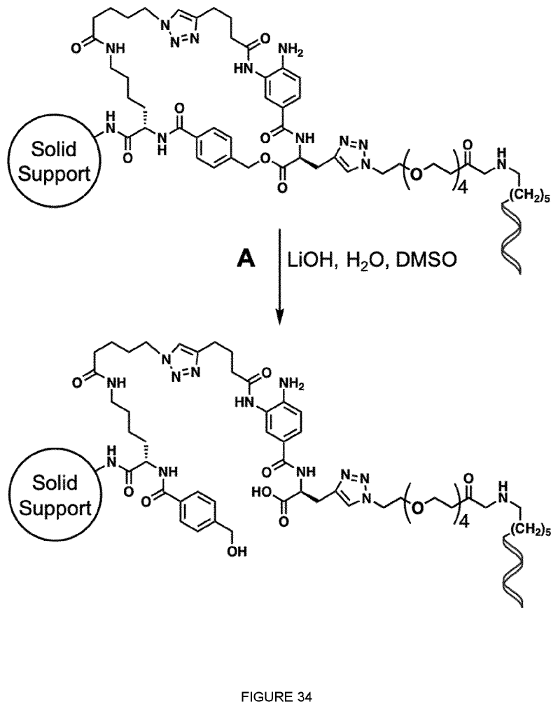

FIG. 35 shows a reaction scheme for the cleavage of the second linker for the self-purification of a nucleic acid encoded library member (Example 7). First, the second linker, Dbz, was activated using isopentyl nitrite (A). In (B), the activated linker was then cleaved in basic conditions in water and DMSO. This released the self-purified nucleic acid encoded library member from solid support.

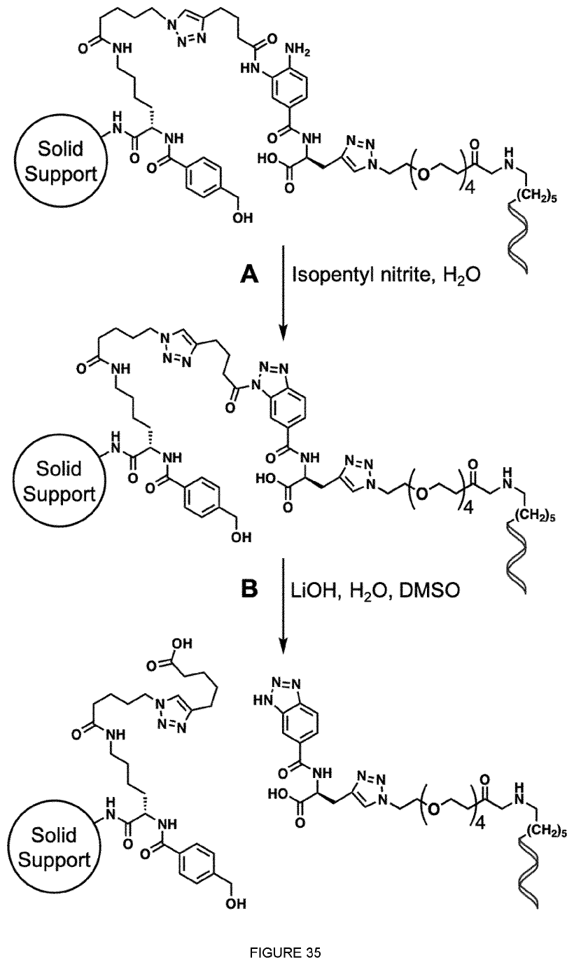

FIG. 36 shows LCMS spectra for the self-purified nucleic acid encoded library member prepared in Example 7. (A) shows the chromatogram for 260 nm absorbance. (B) shows the chromatogram for 280 nm absorbance. (C) shows the mass spectrum of the product peak at 3.97 min. (D) shows the deconvoluted mass spectrum of the product peak at 3.97 min. These data show that the desired self-purified nucleic acid encoded library member was obtained. These data additionally show that the Dbz linker has been activated.

FIG. 37(A) shows a schematic representation for DNA ligation on solid support (Example 8). The code is ligated to the oligonucleotide attached to the solid support by using an adaptor oligonucleotide and T4 DNA ligase. The LCMS chromatogram for 260 nm absorbance (B) was obtained by cleaving the ester linker after the ligation conditions. The ligation product was observed at 4.47 min. Remaining adaptor, code, and non-ligated starting material was additionally observed. (C) shows the deconvoluted mass spectrum for the ligation product peak. The mass for the desired ligation product was observed.

FIG. 38 shows deconvoluted mass spectra for (A) the adaptor oligonucleotide, (B) the code, and (C) the starting material peaks for the LCMS chromatogram shown in FIG. 37B.

FIG. 39 shows a reaction scheme for the transformations performed in Example 9.

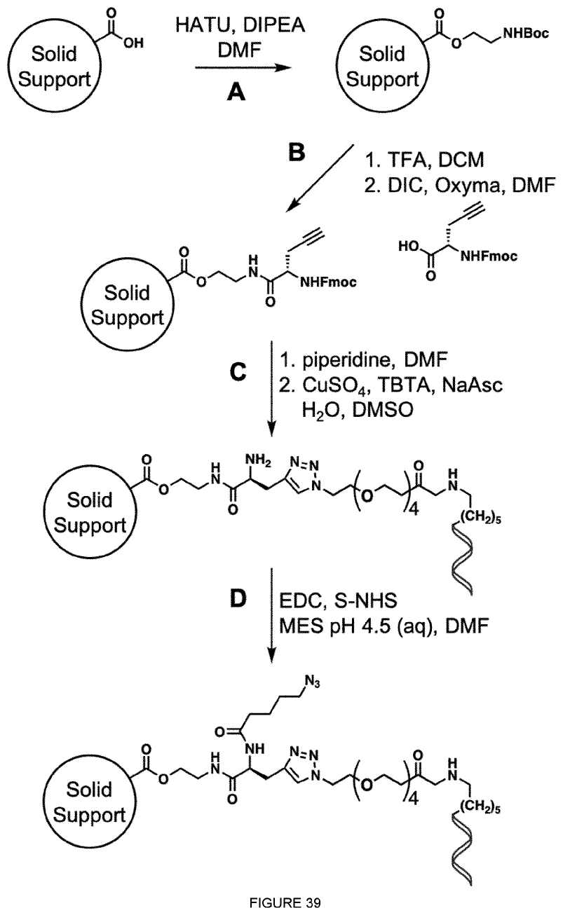

FIG. 40 shows LCMS spectra for samples prepared in Example 9. FIG. 40A shows the chromatogram at 260 nm for the sample prepared in step 9.6, and the chemical structure of the starting material. The major peak observed in FIG. 40A corresponds to the desired starting material. The deconvoluted mass spectrum at the retention time of the major peak in the chromatogram (FIG. 40A) is shown in FIG. 40C. The mass corresponding to the desired starting material is observed. FIG. 40B shows the chromatogram at 260 nm for the sample prepared in step 9.8, and the chemical structure of the desired product. The major peak observed in FIG. 40B corresponds to the desired product. The deconvoluted mass spectrum at the retention time of the major product in the chromatogram (FIG. 40B) is shown in FIG. 40D. The mass corresponding to the desired product is observed. This example shows that chemical transformations can be performed on solid support on DNA with a high conversion.

### DETAILED DESCRIPTION

In some aspects, nucleic acid encoded libraries may be produced as described herein by a method that involves preparing for each library member a solid support compound comprising a scaffold attached to a solid support via a linker, a chemical portion attached to the scaffold, a nucleic portion attached to the scaffold, as well as a cleaving group attached to the chemical portion, scaffold, or nucleic acid portion. The method further comprises reacting the cleaving group with the linker to release the compound from the solid support to form the library member.

Cleavage of the linker by the cleaving group may generate a macrocycle. The macrocycle may comprise the chemical portion and the scaffold. The end of the chemical portion may be covalently connected to the scaffold in the macrocycle by a cyclisation element generated by the reaction of the cleaving group with the activated linker.

Members with a complete chemical portion, scaffold, or coding nucleic acid portion cleaving group (i.e. species in which all of the intended chemical building blocks, coding oligonucleotides or other elements are present) are selectively released from the solid support through cleavage of the linker by the cleaving group. For example, the complete chemical portion may be a chain of linked chemical building blocks (i.e. species in which all of the intended chemical building blocks are present in the chemical portion). The cleaving group does not attach to compounds or members with an incomplete or partial chemical portion, scaffold, or coding nucleic acid portion cleaving group (e.g. unreacted or partially reacted intermediates which are capped), so these compounds or members are not released from the solid support. For example, members with an incomplete or partial chemical portion that does not contain all of the intended chemical building blocks are not released from the solid support. This self-purifies complete compounds or library members and may avoid the need for further purification steps, for example using chromatographic techniques such as HPLC. Self-purification as described herein allows the production of highly pure members and facilitates the production of complex library members with many synthetic steps. The methods described herein may be rapid compared to existing DEL production techniques. They may be amenable to automation and allow the production of high quality DELs that are highly diverse.

The process of separating complete members or compounds from incompletely synthesized compounds which are not cleaved from the solid support may be referred to herein as self-purification. The compound or member which is released from the solid support after cleavage of the linker or linkers and contains the scaffold, the chemical portion, and nucleic acid portion, and may contain parts of linker and parts of cleaving group reacted together in the cleavage reaction, may be referred to as a self-purified compound or member.

In other aspects, nucleic acid encoded libraries may be produced as described herein by a method that involves preparing for each library member a solid support compound comprising a scaffold attached to a solid support via a first linker, a chemical portion attached to the scaffold, a nucleic acid portion attached to the scaffold and a second linker connecting the chemical portion to the solid support. The method further comprises cleaving the first linker, optional washing, and then cleaving the second linker to release the compound from the solid support to form the library member.

The second linker does not attach to compounds or members with an incomplete or partial chemical portion, (e.g. unreacted or partially reacted intermediates which are capped). These compounds or members are attached to the solid support only by the first linker and are released from the solid support by cleavage of the first linker. For example, members with an incomplete or partial chemical portion that does not contain all of the intended chemical building blocks are released from the solid support by cleavage of the first linker and may be removed. Members with a complete chemical portion (i.e. species in which all of the intended chemical building blocks are present) are attached to the solid support by both the first and second linkers. For example, the complete chemical portion may be a chain of linked chemical building blocks (i.e. species in which all of the intended chemical building blocks are present in the chemical portion). These members are selectively released from the solid support through cleavage of the second linker. This self-purifies complete compounds or library members and may avoid the need for further purification steps, for example using chromatographic techniques such as HPLC. Self-purification as described herein allows the production of highly pure members and facilitates the production of complex library members with many synthetic steps. The methods described herein may be rapid compared to existing DEL production techniques. They may be amenable to automation and allow the production of high quality DELs that are highly diverse.

The process of separating complete compounds from incompletely synthesized compounds which are released from the solid support by cleavage of the first linker may be referred to herein as self-purification. The compound or member which is released from the solid support after cleavage of the second linker and contains the scaffold, the complete chemical portion, and nucleic acid portion, and may contain parts of the linker retained following in the cleavage reaction, may be referred to as a self-purified compound or member.

The first and second linker may be orthogonally cleavable. For example, the reaction conditions required to cleave the first linker may be different to the reaction conditions required to cleave the second linker. The first and second linker are therefore independently cleavable by altering the reaction conditions. The first linker 1 must be stable during the synthesis of the solid support compound. The second linker must be stable to the cleavage conditions of the first linker (i.e. the second linker must not be cleaved under conditions that will cause cleavage of the first linker. The cleavage conditions of the first and second linkers must not cause degradation or destruction of the nucleic acid portion.

In some embodiments, the first and/or the second linker may be activated before cleavage.

Suitable first and/or second linkers may include base-cleavable linkers, such as ester linkers, photocleavable, amino (methyl) aniline (MeDbz), amino aniline (Dbz), masked thioester, sulfonamide, oxidatively cleavable, reductively cleavable and enzymatically cleavable linkers.

Suitable first and/or second linkers may include linkers which may be cleaved by nucleophiles. Examples of linkers which may be cleaved by nucleophiles may include thioesters, sulfonamides, benzimidazolones (for example, MeNbz), and benzotriazoles (for example, Dbz linker activated by isopentyl nitrite).

Suitable base-cleavable linkers, such as ester linkers, may be cleaved at high pH. Examples of base-based linkers may include esters, benzyl esters, and 4-(Hydroxymethyl)benzoic acid (HMBA) (Usanov, D. L. et al Nat. Chem. 10, 704-714 (2018); Soural, M. et al Linkers for Solid-Phase Peptide Synthesis. in Amino Acids, Peptides and Proteins in Organic Chemistry vol. 3 273-312 (Wiley-VCH, 2011))

Photocleavable linkers may be cleaved by photoirradiation. Examples of photocleavable linkers may include ortho-nitrobenzyloxy and ortho-nitrobenzylamino, ortho-nitrovetaryl, phenacyl, pivaloyl, benzoin linkers, and other photolabile linkers (Mikkelsen, R. J. T. et al Photolabile Linkers for Solid-Phase Synthesis. (2018) doi:10.1021/acscombsci.8b00028.)

Oxidatively cleavable linkers include geminal diols, such as linkers based on L-tartrate, seramox, and isoseramox linkers (Usanov, D. L. et al Nat. Chem. 10, 704-714 (2018); Pomplun et al Angew. Chemie—Int. Ed. 59, 11566-11572 (2020) These may be cleaved, for example, by sodium periodate.

Other suitable first and/or second linkers are available in the art and include sulfonamide linkers (Mende, F et al. J. Am. Chem. Soc. 132, 11110-11118 (2010)) and cleavable linkers (Scott, P. J. H. Linker Strategies in Solid-Phase Organic Synthesis (2009); Hermanson, G. T., Bioconjugate Techniques: Third Edition (2013); Leriche, G., Chisholm, L. & Wagner, A., Cleavable linkers in chemical biology (2012)). In some embodiments, the first and second linker may be incorporated in a single chemical entity. For example, a single first and second linker may be based on iminodiacetic acid. The first linker may be cleaved in a deprotection-mediated cyclization to yield a diketopiperazine second linker, which is then cleaved at high pH. (Pá Tek, M. & Lebl, M. Safety-Catch and Multiply Cleavable Linkers in Solid-Phase Synthesis. Biopoly vol. 47 (1998); Kočiš, P., Krchňa\acute{k}, V. & Lebl, Tetrahedron Lett. 34, 7251-7252 (1993)).

Other suitable first and/or second linkers may comprise two or more binding groups before attachment to the solid support and to the scaffold and/or to the chemical portion and/or to the nucleic acid portion. For example, a linker may bind to the solid support through a first binding group and may bind to the scaffold and/or to the chemical portion and/or to the nucleic acid portion through a second binding group. The binding groups of linkers may be functional groups. Suitable first and/or second linkers may be homo- or heterobifunctional, referring to their binding groups. Suitable heterobifunctional linkers may include 3-[[(9H-fluoren-9-ylmethoxy)carbonyl]amino]-4-(methylamino)benzoic acid (Fmoc-MeDbz-OH), wherein the carboxylic acid group may for example bind to the solid support and the amine group, after Fmoc deprotection, may for example bind to the scaffold. Other suitable heterobifunctional linkers may include 4-amino-3-[[(9H-fluoren-9-ylmethoxy)carbonyl]amino]benzoic acid (Fmoc-Dbz-OH) and 4-(hydroxymethyl)benzoic acid (HMBA).

A DNA encoded chemical library (DEL) is a collection of chemically diverse library members that each comprise (i) a chemical portion formed from a set of chemically linked chemical building blocks and (ii) a nucleic acid that encodes the set of chemical building blocks that form the chemical portion. The number of different members in a library represents the complexity of a library and is defined by the number of building blocks that form each chemical portion, and by number of different variants of each building block.

A chemical portion is a chemical entity or molecular structure that is displayed by a library member and comprises one, two or more chemical building blocks. The chemical portion is covalently linked to the scaffold and is created by the consecutive covalent addition of the one or more chemical building blocks to form a linear chain or backbone having an end attached to the scaffold and a free end. A cleaving group may be attached to the chemical portion. The different chemical portions displayed by members of a DEL library are formed from different combinations of chemical building blocks. The chemical portions displayed by a DEL may be linear, macrocyclic, bicyclic, multicyclic or branched compounds of different sizes (e.g. Lipinski-like small compounds and larger compounds). In some embodiments, a chemical portion may be any small molecule (i.e. a molecule that has a molecular weight below about 1,000 Daltons). In other embodiments, a chemical portion may be any medium-sized molecule (i.e. a molecule that has a molecular weight below about 5,000 Daltons). Small molecules may be organic or inorganic, isolated (e.g., from compound libraries or natural sources), or obtained by derivatization of known compounds. A chemical portion may be designed or built to have one or more desired characteristics, e.g., capacity to bind a biological target, solubility, availability of hydrogen bond donors and acceptors, rotational degrees of freedom of the bonds, positive charge, negative charge, In vivo stability, cell permeability, and/or oral availability.

Chemical portions displayed in a nucleic acid encoded chemical library may be attached to a single strand of nucleic acid (“single pharmacophore libraries”) or two different strands of nucleic acid hybridised together, one or more building blocks being attached to each strand (“dual pharmacophore libraries”).

The coding nucleic acid portion is a nucleic acid tag or peptide nucleic acid tag which identifies the chemical building blocks in the chemical portion. The coding nucleic acid portion may be a linear nucleic acid molecule which comprises an end connected to the compound or member and a free end. Preferably, the coding nucleic acid portion is attached to the scaffold. In order to record a previously or subsequently introduced chemical building block, the coding nucleic acid portion may be elongated by covalent addition of a coding oligonucleotide to the free end. The coding nucleic acid portion can be used for the identification of the self-purified compound after amplification and nucleic acid sequencing. In some embodiments, a cleaving group may be attached to the nucleic acid portion, for example at the free end.

In the methods described herein, members of a nucleic acid encoded chemical library may be constructed on a solid support by the addition of chemical building blocks and coding oligonucleotides to nascent members.

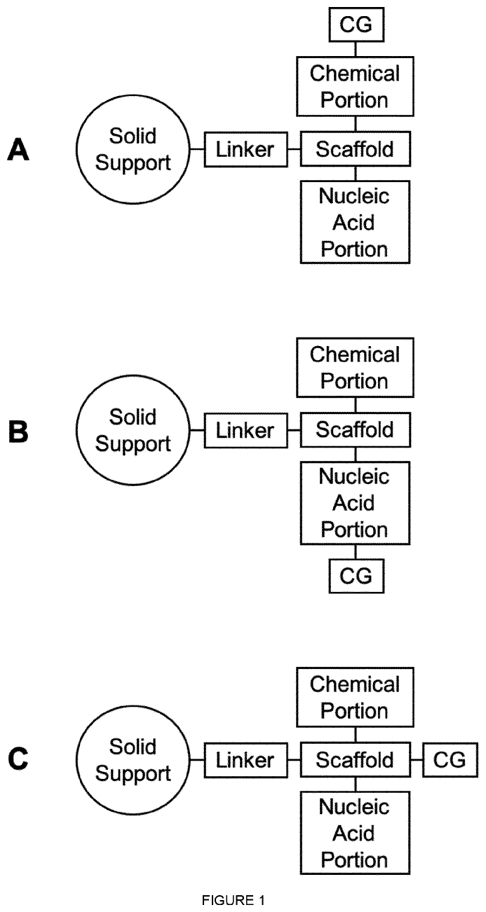

When attached to a solid support, a nascent member may be referred to as a solid support compound. A solid support compound is a compound that comprises a solid support that is connected via the linker to the scaffold, chemical portion, nucleic acid portion, cleaving group and other elements of the nascent member. Examples of solid support compounds according to some embodiments, are shown in FIG. 1. For example, a nucleic acid encoded chemical library may be produced as described herein by a method comprising the steps of;
providing a set of nascent members, each comprising a scaffold connected to a solid support by a linker,
covalently attaching one or more chemical building blocks to the nascent members in the set to form a chemical portion attached to the scaffold, different chemical portions being attached to different nascent members in the set,
covalently attaching to each nascent member coding oligonucleotides encoding the one or more chemical building blocks attached to the nascent member to form coding nucleic acid portions attached to the scaffolds of the nascent members in the set,
attaching a cleaving group to the chemical portions, nucleic acid portions, or scaffolds of the nascent members, and
reacting the linkers and the cleaving groups of each members, such that the linkers are cleaved and the members of the library are released from their respective solid supports.

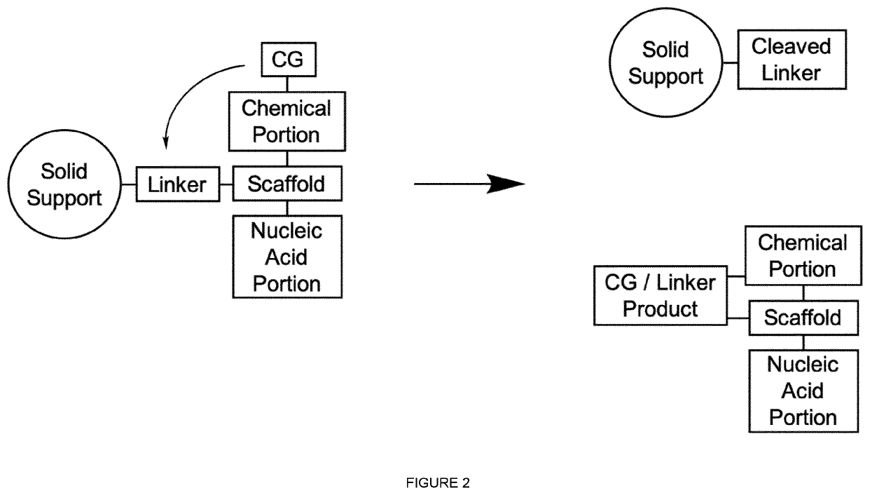

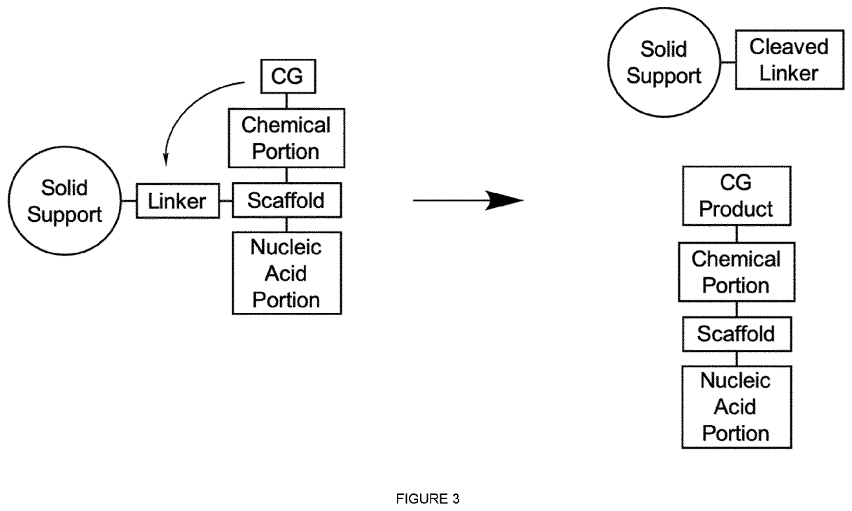

Examples of self-purification reactions (i.e. the reaction of the cleaving group with the linker) are exemplified in FIG. 2 and FIG. 3. In FIG. 2 and FIG. 3, self-purification is exemplified using a solid support compound with the cleaving group attached to the chemical portion.

The reaction of the cleaving group with the linker may result in the release of a compound or member from the solid support that comprises a cleavage moiety (also referred to as a cyclisation element) formed by the reaction of the linker and the cleaving group (i.e. a cleaving group/linker product, see FIG. 2). A portion of the linker (i.e. the cleaved linker) may remain attached to the solid support.

In some embodiments, the cleavage moiety may be connected to the chemical portion where the cleaving group was connected to the chemical portion, and may be connected to the scaffold where the linker was connected to the scaffold. The reaction of the cleaving group with the linker may result in a cyclisation reaction, which yields a cyclic compound attached to the coding nucleic acid portion (FIG. 2). The cyclic compound may comprise the chemical portion, scaffold and cleavage moiety.

In other embodiments, the cleavage moiety may be connected to the chemical portion where the cleaving group was connected to the chemical portion. The reaction of the cleaving group with the linker may not yield cyclisation during the self-purification (see FIG. 3), such that the cleavage moiety is not connected to the scaffold. In some embodiments, all or part of the linker (i.e. the cleaved linker) may remain attached to the scaffold.

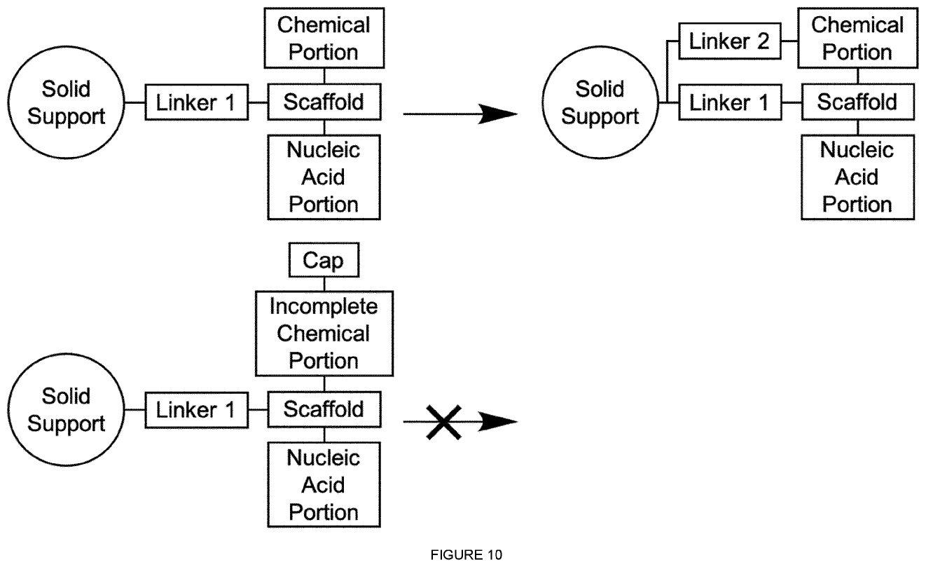

Examples of solid support compounds according to other embodiments are shown in FIG. 10. For example, a nucleic acid encoded chemical library may be produced as described herein by a method comprising the steps of;
providing a set of nascent members, each comprising a scaffold connected to a solid support by a first linker,
covalently attaching one or more chemical building blocks to the nascent members in the set to form a chemical portion attached to the scaffold, different chemical portions being attached to different nascent members in the set,
covalently attaching to each nascent member coding oligonucleotides encoding the one or more chemical building blocks attached to the nascent member to form coding nucleic acid portions attached to the scaffolds of the nascent members in the set,
connecting the chemical portions of the nascent members to the solid supports with second linkers,
cleaving the first linkers and
cleaving the second linkers, such that the members of the library are released from their respective solid supports.

As described above, the first and second linkers may be orthogonally cleavable.

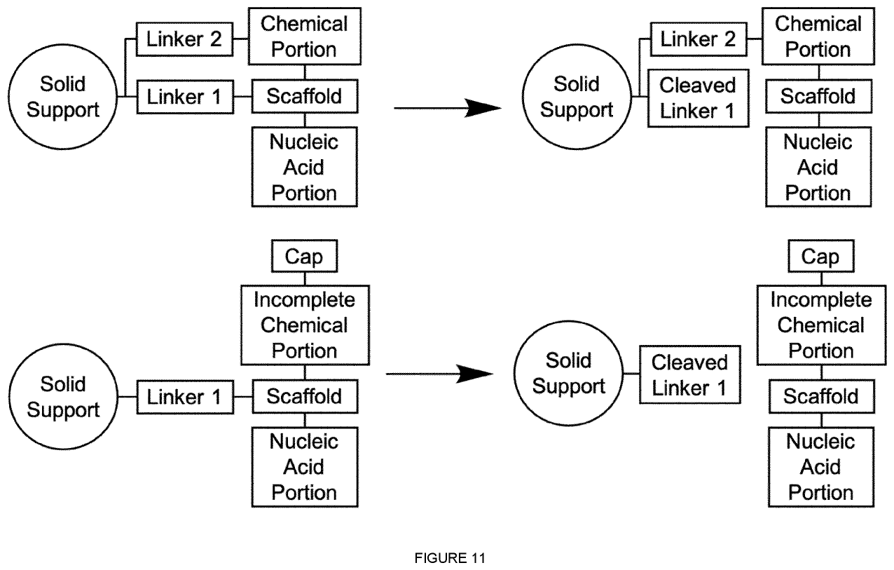

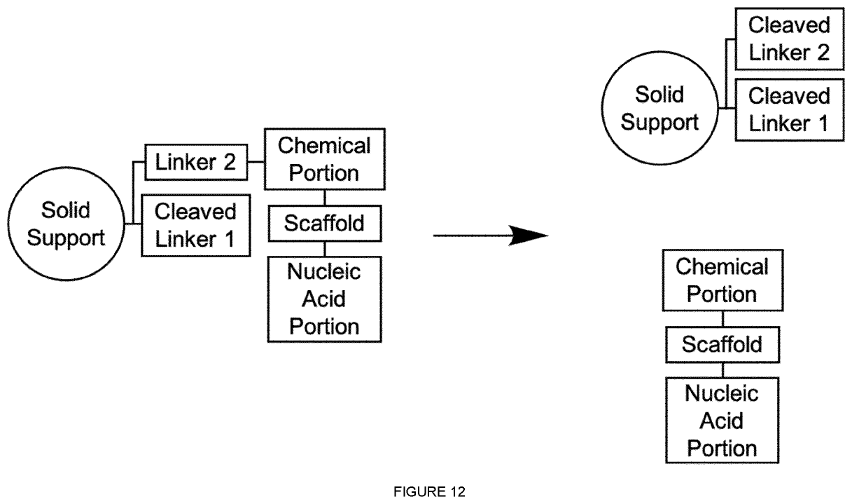

Examples of self-purification reactions (i.e. the reaction of the cleaving group with the linker) are exemplified in FIGS. 10 to 12. In FIGS. 10 to 12, self-purification is exemplified using a solid support compound with a second linker attached to the chemical portion.

The cleavage of the first linker may result in the release from the solid support of compounds or members that have an incomplete or partial chemical portion. These compounds or members may be removed for example by washing. The cleavage of the second linker may result in the release from the solid support of compounds or members that have a complete chemical portion. The released compounds or members may comprise a first and/or a second cleavage moiety (also referred to as a cyclisation element) formed by the cleavage of the first and/or a second linker, respectively. All or part of the first and/or second linker (i.e. the cleaved linker) may remain attached to the solid support.

In some preferred embodiments, the nucleic acid encoded chemical library may be synthesized by consecutive split-and-pool steps, each step comprising the incorporation of a chemical building block to the chemical portion preceded, followed by or simultaneous with the incorporation of a coding oligonucleotide.

Individual nucleic acid encoded compounds or library members may be synthesised by a method which includes first preparing a solid support compound.

In some embodiments, the solid support compound is prepared by attaching a scaffold to a solid support through a linker, attaching a coding nucleic acid portion to the scaffold, attaching a chemical portion to the scaffold, and attaching a cleaving group to the chemical portion, scaffold, or nucleic acid portion. Following the preparation of the solid support compound, the cleaving group is reacted with the linker such that the linker is cleaved and the nucleic acid encoded compound or library member released from the solid support. For example, a nucleic acid encoded compound or library member may be produced as described herein by a method comprising the steps of;
providing a nascent compound that comprises a scaffold connected to a solid support by a linker,
covalently attaching one or more chemical building blocks to the nascent compound to form the chemical portion attached to the scaffold,
covalently attaching coding oligonucleotides encoding the one or more chemical building blocks to the nascent compound to form the coding nucleic acid portion,
attaching a cleaving group to the chemical portion, nucleic acid portion, or scaffold, and
reacting the linker and the cleaving group, such that the linker is cleaved and the self-purified compound or library member released from the solid support.

In other embodiments, the solid support compound is prepared by attaching a scaffold to a solid support through a first linker, attaching a coding nucleic acid portion to the scaffold, attaching a chemical portion to the scaffold, and attaching a second linker to the chemical portion to connect the chemical portion to the solid support. Following the preparation of the solid support compound, the first linker is cleaved, and then the second linker is cleaved and the nucleic acid encoded compound or library member released from the solid support. For example, a nucleic acid encoded compound or library member may be produced as described herein by a method comprising the steps of;
providing a nascent compound that comprises a scaffold connected to a solid support by a first linker,
covalently attaching one or more chemical building blocks to the nascent compound to form the chemical portion attached to the scaffold,
covalently attaching coding oligonucleotides encoding the one or more chemical building blocks to the nascent compound to form the coding nucleic acid portion,
connecting the chemical portion to the solid support with a second linker, and
cleaving the first linker, and
cleaving the second linker, such that the second linker is cleaved and the self-purified compound or library member released from the solid support.

The first chemical building block may be attached to the scaffold before, after or simultaneous with the attachment of the first coding oligonucleotide to the scaffold.

The solid support compound may be prepared, for example, by first attaching the linker to the solid support, and subsequently attaching the scaffold to the linker on the solid support; or by first attaching the scaffold to the linker to form a linker-scaffold conjugate, and subsequently attaching the linker-scaffold conjugate to the solid support.

The coding nucleic acid portion or a fragment of the coding nucleic acid portion may be attached to the scaffold before, after or simultaneous with the attachment of the scaffold to the solid support.

The chemical portion or a fragment of the chemical portion, such as a chemical building block, may be attached to the scaffold before, after or simultaneous with the attachment of the scaffold to the solid support.

A scaffold is a chemical moiety to which the chemical building blocks that form the chemical portion are attached. In some embodiments, preferably, the same chemical moiety forms the scaffold for all of the members of the library. The scaffold may be an at least trifunctional chemical moiety that connects the solid support, the nucleic acid portion, and the chemical portion.

The scaffold may comprise a capture group. The capture group is a reactive chemical group that is capable of reacting with a chemical building block to form a covalent bond linking the chemical building block to the scaffold. This allows the chemical building blocks that form the chemical portion to be attached to the scaffold.

The cleaving group is the chemical reagent or enzyme which can cleave the linker, with or without prior cleaving group activation. In order to provide self-purified compound or member, the cleaving group may in some embodiments, be either covalently or non-covalently linked to the chemical portion, the nucleic acid portion, or the scaffold. The cleaving group may need to be activated after incorporation. The compound or member may be purified by selective release from solid support through the cleavage of the linker by the cleaving group.

In some embodiments, the cleaving group may be attached to the scaffold. Compounds or library members with a complete scaffold and cleaving group may be selectively released from solid support through the cleavage of the linker by the cleaving group.

In other embodiments, the cleaving group may be attached to the chemical portion. Compounds or library members with a complete scaffold, chemical portion, and cleaving group may be selectively released from solid support through the cleavage of the linker by the cleaving group.

In other embodiments, the cleaving group may be attached to the nucleic acid portion, preferably the end of the nucleic acid portion. Compounds or library members with a complete scaffold, nucleic acid portion and cleaving group may be selectively released from solid support through the cleavage of the linker by the cleaving group.

In other embodiments, the scaffold may be connected to the solid support by two different linkers (a first and a second linker), and two different cleaving groups may be present in the solid support compound (a first cleaving group that cleaves the first linker and a second cleaving group that cleaves the second linker). One of the cleaving groups may be attached to the chemical portion or scaffold, and the other cleaving group may be attached to the nucleic acid portion. For example, the method may further comprise;
attaching a first cleaving group to the chemical portion or scaffold,
attaching a second cleaving group to the nucleic acid portion,
reacting the first linker and the first cleaving group, such that the first linker is cleaved, reacting the second linker and the second cleaving group, such that the second linker is cleaved and the compound or member released from the solid support. For example, the first linker may comprise or consist of a substituted-quinoxaline group and the first cleaving group may comprise or consist of an ortho-dithiophenol; and the second linker may comprise or consist of an amino (methyl) aniline (MeDbz) group and the second cleaving group may comprise or consist of a thiol cleaving group.

The selective release from the solid support may be initiated by the presence of both a complete chemical portion and a complete scaffold, both a complete coding nucleic acid portion and a complete scaffold, or more preferably both a complete chemical portion and a complete nucleic acid portion.

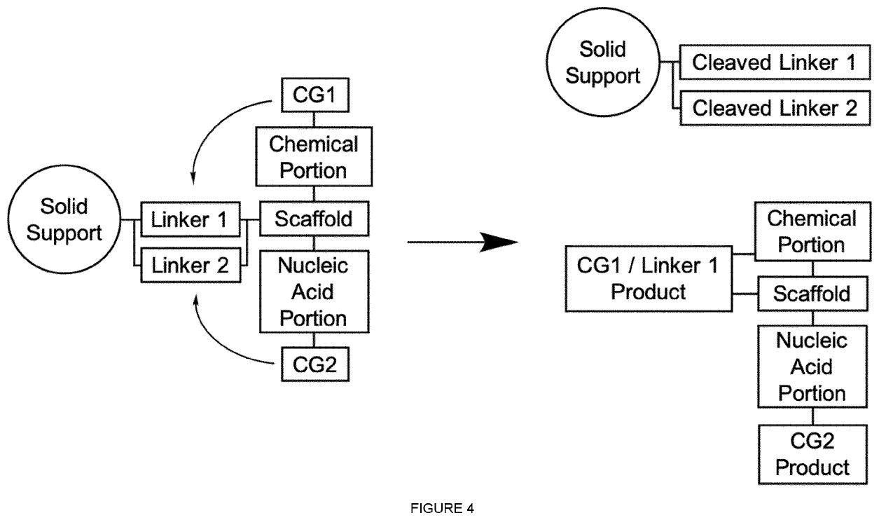

An example of the selective release of a compound or member prepared using two different linkers and two different cleaving groups is shown in FIG. 4. In FIG. 4, the first cleaving group (CG1) is attached to the chemical portion, and the second cleaving group (CG2) is attached to the nucleic acid portion. Two different linkers connect the scaffold to the solid support. CG1 may only react with Linker 1, whereas CG2 may only react with Linker 2. The reaction of CG1 with Linker 1 may occur before, during, or after the reaction of CG2 with Linker 2. Only when both linkers are cleaved is the compound released from the solid support. In the example shown in FIG. 4, the reaction of CG1 with Linker 1 is a cyclisation reaction which results in the formation of a first cleavage moiety (i.e. a CG1/Linker 1 reaction product) which is connected to the chemical portion and the scaffold. CG2 reacts with Linker 2 to yield a second cleavage moiety (i.e. a CG2 reaction product) which is attached to the nucleic acid portion.

In some embodiments, the compound or member may be released from the solid support only when it contains both a complete chain of chemical building blocks of the chemical portion and a complete nucleic acid molecule comprising coding oligonucleotides for all of the chemical building blocks in the chemical portion.

In some embodiments, the cleaving group may be covalently attached to the chemical portion, or scaffold. Preferably, the cleaving group may be covalently attached to the terminal chemical building block in the chemical portion (i.e. at the free end of the chain of chemical building blocks that form the chemical portion). This allows self-purification of members or compounds comprising the whole of the chemical portion.

In other embodiments, the cleaving group may be non-covalently attached to the chemical portion or to the scaffold by hybridization of two oligonucleotides. An anchor oligonucleotide may be covalently attached to the chemical portion or scaffold, preferably the terminal chemical building block of the chemical portion. An auxiliary oligonucleotide which is covalently attached to a cleaving group may then be hybridized to the anchor oligonucleotide. This non-covalently attaches the cleaving group to the chemical portion or scaffold. The cleaving group may then react with the linker to cleave the linker and release the member or compound from solid support. For example, the cleaving group may be attached to the chemical portion or scaffold by a method comprising;
attaching an anchor oligonucleotide to the chemical portion or scaffold,
providing an auxiliary oligonucleotide covalently attached to a cleaving group,
hybridizing the auxiliary oligonucleotide with the anchor oligonucleotide,
reacting the linker and the cleaving group, such that the linker is cleaved.

In some embodiments, the anchor oligonucleotide, which is attached to the chemical portion or scaffold, may be synthesised sequentially on the chemical portion or scaffold, respectively. The anchor oligonucleotide may also be a nucleic acid analogue, such as a peptide nucleic acid. The peptide nucleic acid may, for example, be synthesised sequentially on the scaffold or chemical portion.

Preferably, the anchor oligonucleotide may be covalently attached to the terminal chemical building block of the chemical portion.

The auxiliary oligonucleotide covalently attached to the cleaving group may be a single stranded polynucleotide that is capable of specific hybridisation to the anchor oligonucleotide. Preferably, the auxiliary oligonucleotide covalently attached to the cleaving group comprises 10 or more base pairs.

In some embodiments, the cleaving group may be covalently attached to the nucleic acid portion, for example to the free end of the nucleic acid portion.

In some preferred embodiments, the cleaving group may be linked to the coding nucleic acid portion by hybridization of an auxiliary oligonucleotide covalently attached to a cleaving group to the coding nucleic acid portion in the solid support compound. For example, the cleaving group may be attached to the coding nucleic acid portion by a method comprising;
providing an auxiliary oligonucleotide covalently attached to a cleaving group,
hybridizing the oligonucleotide covalently attached to the cleaving group to the coding nucleic acid portion, and
reacting the linker and the cleaving group, such that the linker is cleaved.

Preferably, the auxiliary oligonucleotide is hybridized to the end portion of the coding nucleic acid (i.e. the region, segment or portion of the nucleic acid which is furthest away or distal from the scaffold). This allows self-purification of members or compounds comprising the whole of the nucleic acid portion.

The auxiliary oligonucleotide may be a single stranded polynucleotide that is capable of specific hybridisation to the coding nucleic acid portion. Preferably, the auxiliary oligonucleotide covalently attached to the cleaving group comprises 10 or more base pairs. Preferably, the auxiliary oligonucleotide covalently attached to the cleaving group hybridises to the end region of the coding nucleic acid portion, for example within 10 bases, 20 bases or 30 bases of the free end of the coding nucleic acid portion.

The coding, attachment, anchor and auxiliary oligonucleotides described herein and the coding nucleic acid portion may be, independently, a natural nucleic acid, such as DNA or RNA, or a nucleic acid analogue, such as a peptide nucleic acid (PNA), a phosphorodiamidate morpholino oligomer (PMO), a phosphorothioate oligomer (PTO), locked nucleic acid (LNA), a glycol nucleic acid (GNA) or a threose nucleic acid (TNA). The auxiliary oligonucleotide may be attached to the cleaving group by any convenient chemistry.

In other embodiments, the scaffold may be connected to the solid support by a first linker and the chemical portion may be connected to the solid support by a second linker. The first and second linker are orthogonally cleavable i.e. the first linker is cleaved under first reaction conditions and the second linker is cleaved under second reaction conditions. For example, the method may further comprise;
exposing or subjecting the solid support compound to the first reaction conditions, such that the first linker is cleaved, and
exposing or subjecting the solid support compound to the second reaction conditions, such that the second linker is cleaved and the compound or member is released from the solid support.

Suitable first and second linkers are described elsewhere herein.

Reactive groups, such as capture groups, binding groups and cleaving groups, may be protected during one or more steps in which the reactive group is not required to react. A reactive group may be conveniently protected by being covalently linked to a protecting group. The reactive group may be deprotected by removing the protecting group before a step in which the reaction of the reactive group is required.

A protecting group is a chemical group that reversibly protects a capture group, binding group, cleaving group or other reactive group described herein against undesirable reactions during one or more steps in which reaction of the capture group, binding group, cleaving group or other reactive group is not required. Commonly used protecting groups are disclosed in Greene, “Protective Groups in Organic Synthesis,” 4th Edition (John Wiley & Sons, New York, 2007). Examples of suitable protecting groups include ester groups (e.g., (methoxyethyl)ester, isovaleryl ester, and -levulinyl ester), trityl groups (e.g., dimethoxytrityl and monomethoxytrityl), xanthenyl groups (e.g., 9-20 phenylxanthen-9-yl and 9-(p-methoxyphenyl)xanthen-9-yl), acyl groups (e.g., phenoxyacetyl and acetyl), silyl groups (e.g., t-butyldimethylsilyl), 2-nitrobenzyl, allyloxycarbonyl-aminomethyl, Allocam oNv, tert-butyl group, tert-butylsulfenyl (StBu) group, sulfonate group 9-fluorenylmethyl group, 9-fluorenylmethoxycarbonyl group or an intramolecular disulfide.

For example, the capture group of the scaffold may be protected e.g. by covalent linkage to a protecting group. The capture group may be deprotected before reaction with the first chemical building block, for example by removing the protecting group.

Protecting groups may be added and removed by any convenient method. Suitable techniques are well established in the art. In some embodiments, the protecting groups may be added and removed by means of a nucleic acid compatible chemical reaction, or more generally, a reaction compatible with the encoding system. In other embodiments, protecting groups may be added or removed by enzymatic transformation. For example, an enzyme-catalysed reaction may be used to protect or deprotect binding groups in the nascent member.

Preferably, the chemical portion is produced by sequentially adding chemical building blocks to the nascent member to produce a linear sequence of chemical building blocks (i.e. a chain) that is attached at a proximal end to the scaffold. The chemical building blocks in the chain may form the chemical portion that is displayed by the member to which it is attached. For example, a first chemical building block may be covalently attached to the capture group of the scaffold. A second chemical building block may be attached to the first chemical building block to form a linear sequence or chain consisting of two chemical building blocks. The chain of chemical building blocks may have a proximal end that is attached to the scaffold and a distal end that is free. The first chemical building block may be at the proximal end position and the second chemical building block may be at the distal end position (i.e. the second chemical building block is the end or terminal chemical building block in the chain). The chain may be extended by sequential attachment of further chemical building blocks to the distal end of the chain e.g. by reaction with the chemical building block at the end position. Following completion of the chain of chemical building blocks, the cleaving group may be attached to the distal end of the complete chain, for example by reaction with the chemical building block at the distal end (the terminal chemical building block).

A chemical building block is a chemical group that forms a structural unit of a chemical portion displayed by a library member. A chemical building block may be any chemical group that comprises one, two, or more binding groups. If a chemical building block is incorporated in a chain of chemical building blocks, this chemical building block may be any chemical group that comprises two or more binding groups.

Preferably, chemical building blocks may comprise two or more binding groups that allow for covalent linkage to the scaffold or to other chemical building blocks. The two or more binding groups may display different or orthogonal reactivity. For example, a chemical building block may comprise a proximal and a distal binding group (e.g. a bifunctional building block). For example, a chemical building block may be covalently attached to the nascent member through the proximal binding group. The distal binding group of the chemical building block may be protected and/or used to attach further chemical building blocks to the nascent member. Each binding group may be a reactive functional group capable of reacting with a binding group from another chemical building block. The proximal and distal binding groups on two different building blocks; or the binding group on a building block and the capture group of the scaffold should be complementary, i.e., capable of reacting together to form a covalent bond. Any reaction compatible with the encoding system, the solid support, and linker integrity may be employed. In some embodiments, any suitable DNA compatible chemistry may be employed, for example amidation, Sonogashira coupling, Suzuki coupling, or copper (I)-catalyzed azide alkyne cycloaddition (CuAAC) or other click reactions. For example, one of the first and second binding groups may be carboxyl group and the other may be an amine group.

Suitable binding groups include carboxylic acids, alkynes, aryl halides, alkyl halides, aldehydes, ketones, nitriles, sulfonyl halides, thiols, alcohols, acetylenes, primary amines, secondary amines, azides, amidines, diamines, epoxides, isocyanates, sulfonamides and boronic acids. Suitable chemical building blocks comprising two or more binding groups include unnatural amino acids, D-amino acids, N-alkylated amino acids, and acid alkynes.

In some embodiments, chemical building blocks may comprise additional binding groups that allow cross-linking between different chemical building blocks within a chain of chemical building blocks, for example to produce macrocyclic, bicyclic or multicyclic chemical entities. For example, a trifunctional building block may be an amino acid with a side-chain with a functionality such as an alkyne, azide, amine, carboxylic acid, thiol, alcohol, or alkyl halide. In some embodiments, chemical building blocks may be crosslinked using a CuAAC reaction or other click reaction, for example between a chemical building block comprising an alkyne binding group and a chemical building block comprising an azide binding group.

In some preferred embodiments, a chemical building block may be covalently attached to the nascent member or compound in a reaction that employs multiple rounds of reagent addition and washing. For example, the solid support may be washed in order to remove the reaction mixture, and a new reaction mixture could be added, for example comprising fresh solvent and reagents. This may be useful for example in driving the reaction of the chemical building block and the nascent member towards completion, increasing the incorporation of the chemical building block and reducing the proportion of unreacted chemical building blocks and nascent members. Multiple rounds of reaction may allow for the incorporation of chemical building blocks which normally are associated with poor reaction yields, such as N-methylated amino acids.

A chemical building block may be covalently connected to the nascent member through its proximal binding group. The distal binding group of the chemical building block may be used to connect further chemical building blocks or the cleaving group to the end of the chain in subsequent steps. During the reaction of the proximal binding group of the chemical building block with the nascent member, the distal binding group of the chemical building block may be protected for example by covalent linkage to a protecting group. Before, for example, the sequential addition of the next chemical building block in a chemical building block chain, the distal binding group may be deprotected, for example by removing the protecting group.

After every chemical building block incorporation step, molecular species of the nascent member which have failed to incorporate the chemical building block (i.e. unreacted members) may be capped. For example, a capping group may be covalently attached to the unreacted capture groups after incorporation of the first chemical building block, and unreacted distal binding groups after incorporation of further chemical building blocks, to prevent the cleaving group from attaching to the unreacted capture or distal binding group.

A capping group is a chemical group that irreversibly caps a reactive group, such as a capture group or a distal binding group described herein and prevents it from taking part in any further chemical reactions. Capping groups are used in the methods described herein to stop unreacted species from one step of a method described herein from reacting in subsequent steps of the method. In particular, capping groups prevent the attachment of the cleaving group to intermediate species. For example, in a chain of chemical building blocks, the distal binding group of the chemical building block at the end of the complete chain remains uncapped and available to react with the cleaving group. This allows the cleaving group to be selectively attached to the end of complete chains of chemical building blocks.

Suitable capping reagents may include monofunctional carboxylic acid derivative reactive groups, such as acetic anhydride, for capping amines; azides for capping alkyne reactive groups; and monofunctional amines for capping carboxylic acid reactive groups.

In some embodiments, capping may not be required. For example, in some embodiments, the distal binding group of a chemical building block (e.g. building block 1) may only be used to react with the subsequent building block (e.g. building block 2) due to the nature of the functional group. Any building blocks thereafter (e.g. building block 3, building block 4, etc.) may not comprise a functional group compatible with binding to the second last building block (e.g. building block 1). Any compounds which failed to incorporate the intermediate building block (e.g. building block 2) therefore may not incorporate any further building blocks.

The same effect as with capping may be achieved thereby.

The methods described herein may be repeated one or more times using different combinations of chemical building blocks to generate a library that comprises diverse members that display different chemical portions. For example, the number, identity and/or order of the chemical building blocks may be different in the chemical portions of different members of the library.

The chemical building blocks and coding oligonucleotides may be conveniently added to nascent library members by a split and pool procedure, as described herein. Alternatively, parallel synthesis for each library member is also possible for sufficiently small libraries.

A split and pool procedure for nucleic acid encoded chemical library synthesis may comprise the steps of;
splitting nascent members or nucleic acid encoded library intermediates into separate compartments,
attaching one or more chemical building blocks,
attaching one or more coding oligonucleotides encoding the chemical building blocks,
and pooling members or intermediates from separate compartments into one or more compartments. This procedure may be repeated one or more times.

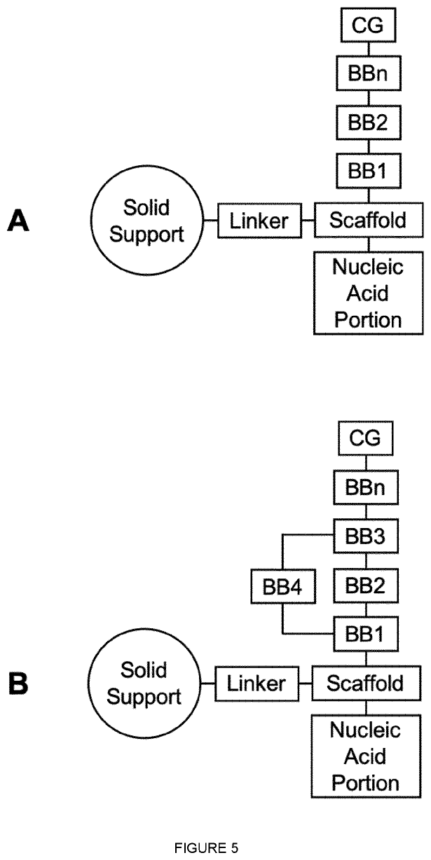

Preferably, the chemical building blocks added to the nascent member form a linear chain of chemical building blocks attached to the scaffold at a proximal end. Compounds and members comprising a complete chain of chemical building blocks can be purified by the method for self-purification described herein. A chain of chemical building blocks is best suited for maximum self-purification of the chemical portion. For chemical building blocks to be incorporated in a chain of chemical building blocks, they must be at least bifunctional (i.e. contain at least two binding groups). The proximal and distal binding groups of a chemical building block may display different or orthogonal reactivity. An example of a solid support compound with a linear arrangement of building blocks is shown in FIG. 5A.

In other embodiments, the chemical building blocks added to the nascent member may form a branched structure attached to the scaffold. The scaffold may be at least tetrafunctional (i.e. it may comprise two or more capture groups) or one or more chemical building blocks may be at least trifunctional to allow for attachment points for two or more additional chemical building blocks to form a branched structure.

In other embodiments, the chemical building blocks added to the nascent member may form a cyclic or macrocyclic structure attached to the scaffold. This may require the cross-linking of a chemical building block with another chemical building block within the chemical portion or with the scaffold. The cyclic structure may be macrocyclic. Additional cross-linking of chemical building blocks with other chemical building blocks or the scaffold may yield chemical portions with bicyclic, macrocyclic or polycyclic structures formed by chemical building blocks attached to the scaffold. An example of a solid support compound with a cyclic and branched arrangement of building blocks formed by crosslinking chemical building block1 and chemical building block3 using an additional building block, chemical building block4 is shown in FIG. 5B.

A capping step may be performed after a synthetic step in the synthesis of the member or solid support compound. Preferably, a capping step may be performed after the addition of each chemical building block and optionally each coding oligonucleotide. Chemical capping steps may include reaction with a monofunctional moiety, or ligation with a non-extendable nucleic acid molecule, such as an oligonucleotide. Functionalised solid support, linker, scaffold, chemical building blocks, the chemical portion, the cleaving group, as well as the coding nucleic acid portion may be capped. A capping step may comprise reacting an unreacted functional group, such as a binding group or capture group with a capping reagent, to form an unreactive capped group. Suitable capping reagents include activated carboxylic acid derivatives.

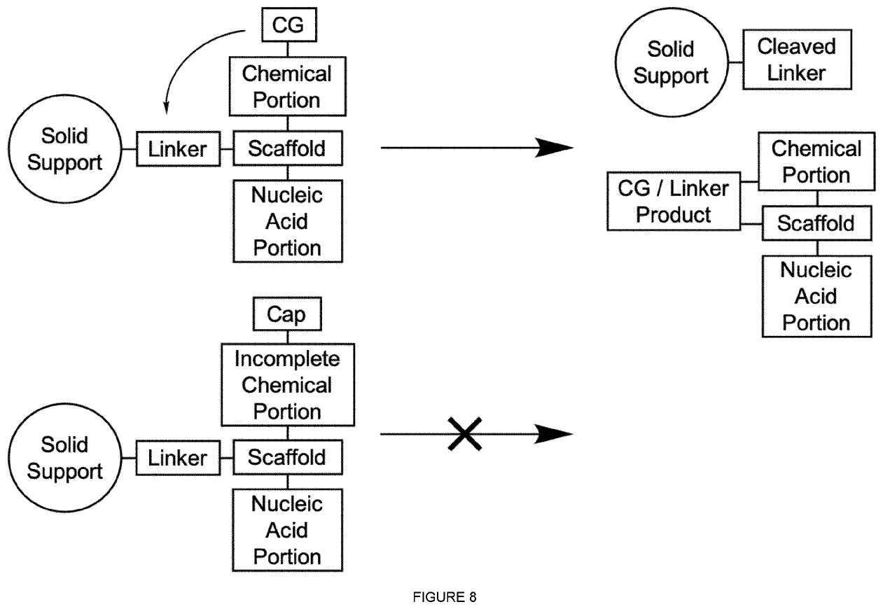

An example of the selective release of complete compounds or members from the solid support is shown in FIG. 8. In the bottom panel of FIG. 8, a compound which is capped in the chemical portion is not released from solid support because it is incompletely synthesised, capped, and therefore does not comprise a cleaving group.

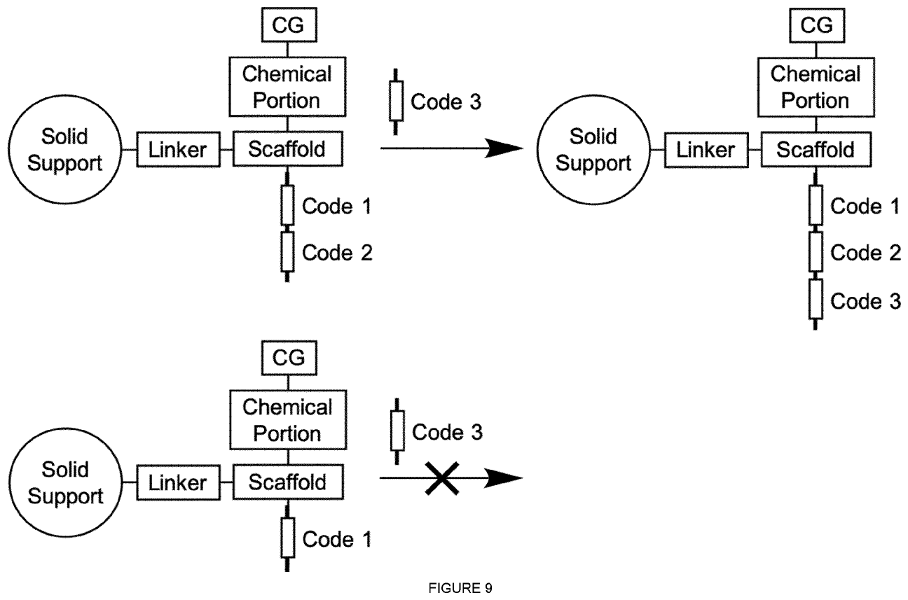

The coding nucleic acid portion may be capped through the use of coding oligonucleotides with ends that are only compatible with the immediately preceding coding oligonucleotide and cannot ligate to other coding oligonucleotides added previously to the nucleic acid portion. An example of the ‘capping’ of the coding nucleic acid portion is shown in FIG. 9. The solid support compound comprising all of the desired nucleic acid codes, Code 1 and Code 2, may be ligated to a further code, Code 3. Code 1 may only be ligated to Code 2, and Code 2 may only be ligated to Code 3. Code 1 may not be ligated with Code 3 as these codes may not contain appropriate sequences for ligation. Therefore, the incompletely synthesised solid support compounds, which, in this example, only comprise Code 1 and not Code 2, may not be ligated to a further code, in this example, Code 3.

Preparation of the member or compound as described herein may include any reaction compatible with the encoding system and the solid support, as well as linker integrity. In some embodiments, possible reactions may include DNA-compatible reactions. Possible reactions may include amide bond formation, Suzuki coupling, Sonogashira coupling, reductive amination, and copper-catalyzed alkyne-azide cycloaddition. DNA compatible reactions are described in literature including (Malone, M. L, Paegel, B. M., ACS Comb. Sci. 2016, 18 (4), 182-187).

In some embodiments, macrocyclization reactions may be used in the reaction of the cleaving group with the linker, the installation of the second linker, the crosslinking of other chemical entities in the solid support compound, or during additional transformations in solution after release from solid support. For example, in some embodiments, the second linker may first be attached to the solid support, and then be connected to the chemical portion in a macrocyclization reaction. Macrocyclization reactions may preferably include reactions which display a high yield, such as copper-catalyzed azide-alykne cyloaddition, for example. Macrocyclization reactions are known in the art and may include reactions described in (Wang, W., Khojasteh, S. C. & Su, D., Mol. (2021); Zhang, R. Y., Thapa, P., Espiritu, M. J., Menon, V. & Bingham, J. P., Bioorg. Med. Chem. (2018)).

A member or compound may be further transformed during and after the preparation. For example, a method may comprise;
crosslinking between two or more of the chemical building blocks, the scaffold, or cleaving group, for example to generate cyclic or multicyclic member or compound,
incorporation of one or more further chemical building blocks, and/or
modification or transformation of one or more chemical building blocks, or the scaffold, linker or cleaving group.

A member or compound may be recaptured onto a new solid support.

In some embodiments, covalent bond formation may be mediated by a nucleic acid templated reaction.

In some embodiments, a macromolecule capable of mediating a transformation, such as an enzyme, may be employed in or after the preparation of a self-purified compound. For example, an enzyme may be recruited by a nucleic acid templated reaction.

A member of a nucleic acid encoded library comprises a nucleic acid portion.

The coding nucleic acid portion may consist of a single-stranded or double-stranded nucleic acid, or a combination of single-stranded and double-stranded nucleic acid. In some embodiments, only one nucleic acid strand may be linked to the scaffold. In other embodiments, both nucleic acid strands of a double-stranded coding nucleic acid portion may be linked to the scaffold.

The scaffold, chemical portion, and/or cleaving group may be encoded by coding sequences in the one or more nucleic acid strands of the nucleic acid portion. Elongation of the coding nucleic acid portion to incorporate a coding sequence may be performed by enzymatic ligation of a coding oligonucleotide; chemical ligation of a coding oligonucleotide; elongation across a coding oligonucleotide template using a polymerase enzyme; or a combination of any of these three methods.

A synthetic step of producing or transforming the scaffold, chemical portion, or cleaving group may be preceded or followed by addition of coding oligonucleotides to the nucleic acid portion. Coding sequences may be present on only one nucleic acid strand, or may be present on two nucleic acid strands. Coding sequences on one nucleic acid strand may be conveniently amplified, for example by PCR. Coding sequences may be on one or two nucleic acid strands and may be transcribed onto one nucleic acid strand which can be PCR amplified. A coding sequence may encode the scaffold, one or more building blocks, a cleaving group, one or more linkers or a combination thereof.

A synthetic step of producing, extending or transforming the scaffold, chemical portion, or cleaving group may preceded or followed by addition of coding sequences to the coding nucleic acid portion.

In some embodiments, only chemical building blocks may be encoded. In other embodiments, the scaffold, linker, cleaving group, or other entities may also be encoded. Although chemical building blocks which are encoded are described below, it is to be understood that also other entities may be encoded in the same way that the below described chemical building blocks are encoded.

Preferably, a member of a nucleic acid encoded library comprises a coding nucleic acid portion that encodes all of the chemical building blocks in the chemical portion that is attached to the member. Sequencing of the coding nucleic acid portion attached to a member allows the identification of the chemical building blocks in the chemical portion that is displayed by the member.

Nascent members or compounds may comprise an attachment oligonucleotide (also referred to as a headpiece). In some preferred embodiments, the attachment oligonucleotide is attached to the scaffold of the nascent member.

The attachment oligonucleotide is a nucleic acid to which coding oligonucleotides encoding chemical building blocks are attached to form the nucleic acid portion. The attachment oligonucleotide may have the same nucleotide sequence in different members of the library (i.e. a constant nucleotide sequence). The combination of coding oligonucleotides and hence the sequence of the coding nucleic acid portion may be different in different members of the library.

The attachment oligonucleotide may have an end that is attached to the nascent binding member and a free end to which coding oligonucleotides are attached. The free end of the attachment oligonucleotide may be compatible with the attachment of coding oligonucleotides. For example, the free end may comprise a short 5′ or 3′ overhang (a “sticky end”) to facilitate ligation.

The attachment oligonucleotide may be a natural nucleic acid, such as DNA or RNA, or it may be a nucleic acid analogue, such as a peptide nucleic acid (PNA), a phosphorodiamidate morpholino oligomer (PMO), a phosphorothioate oligomer (PTO), a locked nucleic acid (LNA), a glycol nucleic acid (GNA) or a threose nucleic acid (TNA).

A first coding oligonucleotide encoding the first chemical building block may be attached to the attachment oligonucleotide. Suitable techniques for attachment of oligonucleotides are well-established and include enzymatic ligation. Subsequent coding oligonucleotides encoding the second and further chemical building blocks may be attached to the previous coding oligonucleotide to form a coding nucleic acid comprising coding oligonucleotides for the chemical building blocks in the chain attached to the member.

In some embodiments, the attachment oligonucleotide may be double-stranded.

A double stranded attachment oligonucleotide may be formed from the intramolecular hybridisation of a single nucleotide strand (i.e. a hairpin) or may be formed from the intermolecular hybridisation of two separate nucleotide strands. The double stranded nucleotide sequence may be denatured to produce a single stranded nucleic acid before cleavage of the linker. Hybridisation of the single stranded nucleic acid of a first released member with the single stranded nucleic acid of a second released member may be useful for example, in producing the members of an ESAC library. Alternatively, a double stranded attachment oligonucleotide in which the two oligonucleotide strands are covalently linked may be employed.

Double-stranded coding oligonucleotides may be attached to a double stranded attachment oligonucleotide by ligation using a ligase, such as T4 DNA ligase, in accordance with standard techniques.

In other embodiments, the attachment oligonucleotide may be single-stranded. Single-stranded coding oligonucleotides may be attached to the attachment oligonucleotide by splint ligation using an adaptor oligonucleotide, in accordance with standard techniques.

A coding oligonucleotide is a nucleic acid molecule that contains a nucleotide coding sequence that encodes a chemical building block and optionally the cleaving group, scaffold and/or linker. The coding sequence (or coding region) can be any sequence of nucleic acid bases that is uniquely associated with a particular chemical building block. This allows the identity of the chemical moiety to be determined by sequencing or otherwise ‘reading’ the coding sequence.

A coding sequence contains sufficient nucleotides to uniquely identify the chemical building block for which it is coding. For example, if the chemical portion has 20 variants, the coding sequence needs to contain at least 3 nucleotides (4²=16, 4³=64). The coding sequence may be longer than necessary. The benefit of employing coding sequences that are longer than necessary is that they provide the opportunity to differentiate codes by more than just a single nucleotide difference, which gives more confidence in the decoding process. For example, a first chemical building block from a population of 20 different chemical building blocks (20 compounds) may be encoded by 6 nucleotides, and a second chemical building block from a population of 200 different moieties may be encoded by 8 nucleotides. The size of the coding sequence therefore depends on the number of chemical building blocks to be encoded (i.e. the number of different chemical building blocks in the library). A sequence of nucleotides and/or its complement may be used as a coding sequence to encode a chemical building block. Suitable sequences for encoding chemical building blocks in a library are well-known in the art.

The coding sequences of the coding oligonucleotides may be flanked by constant regions. The constant regions may be of sufficient length to allow an efficient hybridization and ligation, for example 2-20 bases, preferably 9-15 bases.

Coding oligonucleotides are added in a sequential fashion to the member or compound, concurrently with the incorporation of the building blocks, resulting in a nucleic acid molecule (i.e. nucleic acid portion) containing a linear series of coding oligonucleotides that encode the combination of chemical building blocks that is present in the member or compound. The first coding oligonucleotide that encodes the first chemical building block may be linked to the attachment oligonucleotide and further coding oligonucleotides may each be linked to the preceding coding oligonucleotide in the series to form a nucleic acid molecule (i.e. the nucleic acid portion). The sequence of the nucleic acid portion of a library member encodes the chemical building blocks of the library member. Sequencing the coding nucleic acid portion thus allows the chemical building blocks of a member to be identified.

The first chemical building block and coding oligonucleotide may be attached to the nascent member or compound by a method comprising;
covalently attaching a first chemical building block to the scaffold of the nascent member or compound, and
covalently attaching a coding oligonucleotide encoding the first chemical building block to the attachment oligonucleotide of the nascent member.

After the reaction, unreacted species may be removed by washing or capped to prevent further reactions. For example, the method may further comprise capping scaffolds not covalently attached to the first chemical building block. Suitable methods of capping are described above.

The first chemical building block may be protected to prevent unwanted reactions. For example, the distal binding group of the first chemical building block may be covalently linked to a protecting group. The first chemical building block may be subsequently deprotected, for example after attachment of the coding oligonucleotide. A method may comprise removing the protecting group from the distal binding group of the first chemical building block attached to the scaffold. This allows the addition of a second chemical building block.

In some embodiments, the first chemical building block may be attached to the nascent compound or member by a method comprising;
covalently attaching a first chemical building block to the scaffold of the nascent member, wherein the first chemical building block comprises a proximal binding group that reacts with the capture group of the scaffold to form a covalent linkage and a protected distal binding group,
capping unreacted capture groups not attached to the first chemical building block,
covalently attaching a first coding oligonucleotide encoding the first chemical building block to the attachment oligonucleotide of the nascent member to form a coding nucleic acid, and
deprotecting the distal binding group of the first chemical building block.

Following deprotection of the distal binding group, a second chemical building block may be attached to the nascent member by a method comprising;
covalently attaching a second chemical building block to the first chemical building block of the nascent member, and
covalently attaching a coding oligonucleotide encoding the second chemical building block to the nascent member.

After the reaction, unreacted species may be removed by washing or capped to prevent further reactions. For example, the method may further comprise capping the distal binding group of any chemical building blocks not covalently attached to the further chemical building block.

The further chemical building block may be protected to prevent unwanted reactions. For example, the distal binding group of the further chemical building block may be covalently linked to a protecting group. The further chemical building block may be subsequently deprotected, for example after attachment of the coding oligonucleotide. A method may comprise removing the protecting group from the distal binding group of the further chemical building block attached to the scaffold. This allows the attachment of additional chemical building blocks or the cleaving group.

In some embodiments, a further chemical building block may be attached to the nascent member by a method comprising;
covalently attaching a further chemical building block to the chain of chemical building blocks, wherein the further chemical building block comprises proximal and distal binding groups, and said proximal binding group of the further chemical building block reacts with the distal binding group of the chemical building block at the distal end position to form a covalent linkage, such that the further chemical building block is added to the distal end of the chain of chemical building blocks,
covalently attaching a further chemical building block to the chain of chemical building blocks, wherein the further chemical building block comprises a protected distal binding group and a proximal binding group that reacts with the distal binding group of the chemical building block at the distal end of the chain to form a covalent linkage,
capping unreacted distal binding groups of chemical building blocks at the distal end not attached to the further chemical building block,
covalently attaching a further coding oligonucleotide encoding the further chemical building block to the coding nucleic acid, and
deprotecting the distal binding group of the further chemical building block.

Covalent attachment of the further coding oligonucleotide may be performed before, after or at the same time as deprotection of the distal binding group.

This process may be repeated one or more times to incorporate multiple further chemical building blocks to produce the chemical portion. For example, the chemical portion may comprise a chain of 1, 2, 3, 4, 5 or more chemical building blocks. In some preferred embodiments, the chemical portion may comprise up to 20 chemical building blocks. In other preferred embodiments, the chemical portion may comprise up to 10 chemical building blocks. In other preferred embodiments, the chemical portion may comprise up to 6 chemical building blocks.

In some embodiments, the chemical building block at the end of the chain of chemical building blocks (i.e. the terminal chemical building block) may comprise a protected distal binding group, for example a distal binding group that is covalently linked to a protecting group. A method may comprise removing the protecting group from the distal binding group of the chemical building block at the end of the chain of chemical building blocks before attachment of the cleaving group.

A solid support is an insoluble body which presents a surface on which the nascent member or compound can be attached during production as described herein. Examples of suitable supports include resins, beads, nanoparticles and polymers such as polystyrene-polyethylene glycol (PEG) composites, PEG and poly-ε-lysine (ε-PL) (see for example Albericio F (2000). Solid-Phase Synthesis: A Practical Guide. Boca Raton: CRC Press). Conveniently, the support may be in form of particles, such as beads. In some embodiments, the solid support may be a bead of a grafted copolymer consisting of a polystyrene matrix grafted with poly (ethylene glycol) (PEG). Solid supports may be produced using standard techniques or obtained from commercial suppliers (e.g. Tentagel®, Rapp Polymere GmbH, DE). The separation of the compounds on solid support from a solution may be achieved by any convenient method, such as filtration, by magnetic interactions (for magnetic beads), by centrifugation, etc.

Other suitable solid supports may include polystyrene beads, crosslinked polystyrene beads, polymer beads, glass beads, coated glass beads, controlled-pore glass beads, beaded controlled-pore glass beads, silica microparticles, coated silica microparticles, iron oxide particles, coated iron oxide particles, PEGA (polyethylene glycol-acrylamide) resin, and other commercially available or custom synthesized solid supports of different sizes, or combinations thereof. Suitable solid supports may be magnetic. Examples of magnetic solid supports include Magnefy™ and ProMag 1® microspheres (Bangs Laboratories, Inc.). Examples of solid supports may include co-polymers, such as acrylamide-PEG co-poymer, polymer particles which additionally comprise a material which is paramagnetic or ferromagnetic, core-shell particles, porous particles, non-porous particles, or other organic chemical materials in combination with a ferromagnetic material. Other suitable solid supports are known in the art (see for example, Pon, R. T. Curr. Protoc. Nucleic Acid Chem. (2000); Chaudhuri, R. G. & Paria, S., Chem. Rev. (2011); Wu, W., He, Q. & Jiang, C. Nanoscale Res. Lett. (2008); Hermanson, G. T., Bioconjugate Techniques: Third Edition (2013)).

In some embodiments, a binding entity may be used for capture onto solid support. For example, a small organic or inorganic entity may be used for capture onto a solid support to allow the physical separation of compounds bond to the solid support from a solution. Suitable small organic or inorganic binding entities may include biotin and quantum dots, such as magnetic quantum dots. In some embodiments, one or more steps in the synthesis of a nucleic acid encoded compound or library as described herein may be performed in solution before or after capture onto a solid support.

In some embodiments, solid supports may be additionally functionalized by a linear (for example, polyethylene glycol (PEG) spacer) or dendrimer structure (for example, polyamidoamine (PAMAM) dendrimer). Dendrimers and spacers are described in literature including (Hermanson, G. T., Bioconjugate Techniques: Third Edition (2013)). In some embodiments, the solid support surface may be modified by a small molecule. In some embodiments, for example, the small molecule may connect a part of the solid support with the first and/or second linker.

Preferably, the collection of solid support particles can be readily suspended in a solution to allow for splitting and pooling, if this is desired. In some embodiments, a small solid support particle size may be preferable for the facile synthesis of a library with a large number of distinct members. For example, microparticles or nanoparticles may be used.

The solid support allows the bound member or compound to be washed after one or more steps of construction as described herein to remove unbound reactants. Only complete members are capable of self-release. This may allow the separation of pure library members from incompletely synthesized library members, allowing the production of DELs with high purity. In some embodiments, the separation of the released members in solution from species that remain attached to the solid support allows the enrichment or purification of complete members, allowing the production of DELs with high purity. In other embodiments, the separation of the released members in solution is preceded by a step which may only or preferentially release incompletely synthesized species from the solid support. The subsequent release of members that remain attached to the solid support allows the enrichment or purification of complete members, allowing the production of DELs with high purity.

In some embodiments, members or compounds as described herein are released from the solid support by the reaction of the cleaving group and the linker. An electrophilic cleaving group and a nucleophilic linker may be employed or more preferably a nucleophilic cleaving group and an electrophilic linker. In some embodiments, the reaction between the linker and the cleaving group may be a substitution reaction, substituting the solid support with the cleaving group. The substitution reaction may be a nucleophilic substitution reaction, for example a nucleophilic aromatic substitution reaction. Other suitable reactions between the linker and the cleaving group may include metal-catalysed reactions or metathesis reactions.

In some preferred embodiments, one of the linker and the cleaving group may be a thiol or selenothiol group and the other may be a carbonyl group. This reaction may result in the formation of a thioester intermediate. The thioester intermediate may subsequently be cleaved intermolecularly or intramolecularly, which may result in the irreversible cyclisation of the library member (FIG. 2).

A linker is a cleavable chemical moiety that may, in some embodiments, connect the scaffold to the solid support. The linker may connect the scaffold directly to the solid support or indirectly, for example through an anchor. The linker may be cleaved through a chemical reaction mediated by specific reagents (e.g. cleaving group) or reaction conditions.

In other embodiments, a first and a second linker may be present. The first linker may be a cleavable chemical moiety that covalently connects the scaffold to the solid support. The second linker may be a cleavable chemical moiety that covalently connects the chemical portion to the solid support. The first and second linkers may be orthogonally cleavable i.e. the first linker may be cleaved by specific reagents (e.g. cleaving group) or reaction conditions that do not cleave the second linker.

In some embodiments, a linker may not require further transformation or activation after attachment to the solid support and the scaffold before reaction with the cleaving group. The linker may be incorporated into the nascent member in an active state (i.e. the linker is in a form that is reactive to the cleaving group). Suitable linkers include substituted quinoxalines or derivatives thereof, which may be cleaved by an ortho-dithiophenol cleaving group without further transformation or activation.

A substituted-quinoxaline may comprise a quinoxaline group with one or more substitutions, for example substitutions at positions 2, 3 and 5 or positions 2, 3 and 6. In some embodiments, position 2 of a substituted quinoxaline may be a halogen, such as F, Cl, Br or I, preferably CI, or an electron withdrawing group; position 3 may be —SR, —OR or —NR and position 5 or 6 may be —COOR, —CONR, or alkyne. In other embodiments, position 2 of a substituted quinoxaline may be —SR, —OR or —NR; position 3 may be a halogen, such as F, Cl, Br or I, preferably CI, or an electron withdrawing group and position 5 or 6 may be —COOR or —CONR, or alkyne. R may be independently selected from a hydrogen atom, or a C₁₋₆ alkyl group, C₆₋₂₀ aryl, a C₁₋₆ alkoxy group, a C₁₋₆ acyloxy group, or a C₁₋₆ reverse ester group; any of which may be linear or branched and optionally substituted. Examples of suitable substituted-quinoxalines may include 3-chloro-2-((2-hydroxyethyl)thio)quinoxaline-6-carboxylic acid; 3-chloro-2-(4-(hydroxymethyl)phenoxy)quinoxaline-6-carboxylic acid; and N-(3-aminopropyl)-3-chloro-2-(4-(hydroxymethyl)phenoxy)quinoxaline-6-carboxamide.

In other embodiments, the linker may require activation after attachment to the solid support and the scaffold and before reaction with the cleaving group i.e. the linker may be an activatable linker. The linker may be incorporated into the nascent member in an inactive state, (i.e. the linker is in a form that is not reactive to the cleaving group). Activation of the linker converts it from a non-activated form into an activated form. The activated form of the linker is selectively cleaved by the cleaving group, whereas the non-activated form of the linker is not cleaved by the cleaving group. For example, the inactive form of the linker may comprise a protecting group and the linker may be activated by removal of the protecting group. A method described herein may comprise activating the activatable linker. In embodiments in which the cleaving group requires activation, the linker may be activated before, after, or simultaneously with the activation of the cleaving group.

Suitable activatable linkers include masked thioesters, such as N-alkyl cysteine. Masked thioesters may be activated to produce a thioester that may be cleaved by a thiol cleaving group (native chemical ligation). The thiol in the masked thioester may for example be protected by a tert-butyl group, allyloxycarbonylaminomethyl group, 2-nitroveratryl group, 9-fluorenylmethyl group, or as an S-sulfonate. After deprotection of the masked thioester, the cysteine derivative may undergo an N to S rearrangement upon deprotection of the thiol to give a thioester.

Other suitable activatable linkers include diaminobenzoyl groups or derivatives thereof, such as methyl diaminobenzoyl groups. For example, the activatable linker may be amino (methyl) aniline (MeDbz). MeDbz may be activated by reaction with para-nitrophenyl choloroformate to produce N-acyl N′-methyl benzimidazoline (MeNbz), which may be cleaved by a thiol cleaving group. Other examples of suitable activatable linkers include 3,4-diaminobenzoic acid (Dbz), and derivatives thereof, which may be activated with isopentyl nitrite to produce a benzotriazole derivative (Selvaraj, A. et al, Chem. Sci., 2018, 9, 345-349).

Other suitable activatable linkers include enzyme substrates. For example, the activated linker may be an oligonucleotide that is cleaved by a nuclease cleaving group or a peptide that is cleaved by a peptidase.

In some embodiments, a linker is cleaved by a cleaving group to release the solid support compound. The cleaving group is a reactive chemical group, reagent, or enzyme that is capable of reacting with the linker to cleave the linker and release the nascent member from the solid support. In order to provide self-purified compound, the cleaving group may be either covalently linked to or reversibly associated with the chemical portion, the nucleic acid portion, or the scaffold. For example, the cleaving group may be attached to the distal end of a chain of chemical building blocks after completion of the chemical portion. The cleaving group may be attached to the distal binding group of the chemical building block at the distal end position of the chain of chemical building blocks in the chemical portion. In other embodiments, the cleaving group may be attached to any position in the chemical portion, to the scaffold, or to the nucleic acid portion.

In some embodiments, the cleaving group does not require further transformation or activation after attachment to the chemical portion, scaffold, or the coding nucleic acid portion and before reaction with the linker.

In other embodiments, the cleaving group requires activation after attachment to the chemical portion, scaffold, or the coding nucleic acid portion and before reaction with the linker i.e. the cleaving group may be an activatable cleaving group. The cleaving group may contain a functional group which is protected upon integration of the cleaving group into the member or compound, and which after deprotection cleaves the linker. For example, the cleaving group may comprise a protecting group and may be activated by removing the protecting group.

In some preferred embodiments, the cleaving group may be protected. For example, it may be covalently linked to a protecting group. A protected cleaving group may be inactive and may be activated by deprotection. A method may further comprise deprotecting the cleaving group, for example by removing the protecting group. The cleaving group may be deprotected before, after or simultaneously with a potential activation of the linker. Suitable protecting groups are described above.

A cleaving group may preferably be at the end of the chain of chemical building blocks that form the chemical portion attached to the scaffold (i.e. the distal end of the chemical portion). Alternatively, a cleaving group may be attached to a chemical building block within the chain other than the terminal chemical building block or incorporated between two chemical building blocks in the chain.

In some embodiments, a compound or member may comprise multiple different cleaving groups. For example, two different or orthogonal cleaving groups may be attached to two different positions in the chemical portion.

The choice of cleaving group will depend on the activatable linker.

Suitable cleaving groups may comprise or consist of a thiol. In some embodiments, a thiol cleaving group may be used to cleave a thioester linker, for example, a thioester linker produced by activation of a masked thioester, or masked N-alkyl cysteine; or a diaminobenzoyl linker such as the MeNbz linker, for example a MeNbz linker produced by activation of MeDbz. The thiol may be protected during the incorporation into the solid support compound.

Other suitable cleaving groups may comprise or consist of a selenothiol, which may be protected during incorporation into the member or compound on the solid support. The selenothiol may display similar reactivity with a linker compared to a thiol. The selenothiol may be protected during the incorporation into the solid support compound. For example, the cleaving group may be cysteine or a derivative thereof, or selenocysteine, or a derivative thereof. An amine in the cleaving group in addition to a thiol or selenothiol may result in intramolecular cleavage of the thioester or selenoester formed after reaction of the cleaving group with the linker by the amine.

In some embodiments, the cleaving group may comprise multiple thiol or selenothiol groups.

Other suitable cleaving groups may comprise or consist of an ortho-dithiophenol. An ortho-dithiophenol cleaving group may be used to cleave a substituted quinoxaline linker. Substituted quinoxaline linkers are described in more detail above.

Other suitable cleaving groups may comprise or consist of an enzyme. An enzyme cleaving group may be used to cleave a linker comprising an enzymatically cleavable structure. For example, a nuclease cleaving group may be used to cleave a polynucleotide linker and a peptidase may be used to cleave peptide linker.

Other reactions may be used for the cleavage of the linker by the cleaving group. Reactions which are compatible with the selective reaction of a cleaving group and a linker as described herein may be employed. For example, the linker may be an N- and O-substituted hydroxylamine, and the cleaving group may comprise an alpha-ketoacid group. In another example, the linker may comprise a carboxylic acid ortho-hydroxybenzaldehyde ester, which is further derivatized on the aromatic ring, and the cleaving group may comprise serine, or a derivative thereof, or threonine, or a derivative thereof, linked to the chemical portion, the scaffold, or the coding nucleic acid portion via its carboxyl group.

The same or different types of protecting groups may be used for the scaffold, chemical building blocks and the cleaving group, as long as they do not undesirably interfere in the synthesis of a self-purified compound or member.

For example, a thiol cleaving group may be protected by covalent linkage to an allyloxycarbonyl-aminomethyl group, o-nitrobenzyl group, 2-nitroveratryl (Nv) group, tert-butyl (tBu) group, 9-fluorenylmethyl group, through formation of a S-sulfonate, or through a disulfide bond or selensulfide bond. Preferably, the protection through a disulfide or selensulfide bond may be intramolecular.

A selenothiol cleaving group may be protected through a selensulfide or a diselenide bond.

An ortho-dithiophenol cleaving group may be protected by covalent linkage by intermolecular disulfide bond formation of each thiol. For example, thiol groups in the ortho-dithiophenol cleaving group may each be protected by a S-tert-butyl group. In some embodiments, the thiols may be protected by other protecting groups mentioned above.

In some embodiments, the protecting group of a functionality in the cleaving group may be photolabile and may be attached to the cleaving group by a photolabile bond. The protecting group may be removed by the application of light to cleave the photolabile bond and activate the cleaving group. The cleaving group may be deprotected before, after or simultaneously with a potential activation of the linker. Suitable photolabile protecting groups include 2-nitrobenzyl groups, such as the 2-nitroveratryl group.

In other embodiments, the solid support member may comprise first and second linker that are independently cleavable by exposing the member to suitable conditions, without the requirement for a cleaving group. The first linker may connect the scaffold to the solid support. The second linker may connect the chemical portion to the solid support. Cleavage of the first linker may release members that are not also connected through a second linker (i.e. members with an incomplete chemical portion).

One enzyme or different enzymes may mediate one reaction or multiple reactions involved in the production of a self-purified compound or member as described herein.

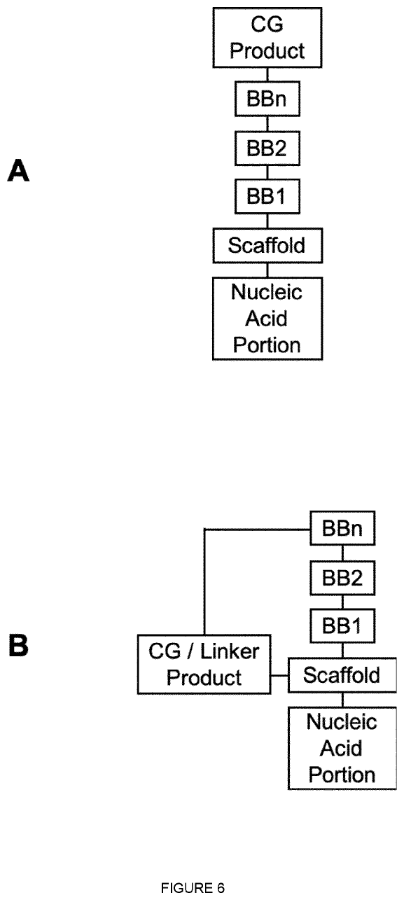

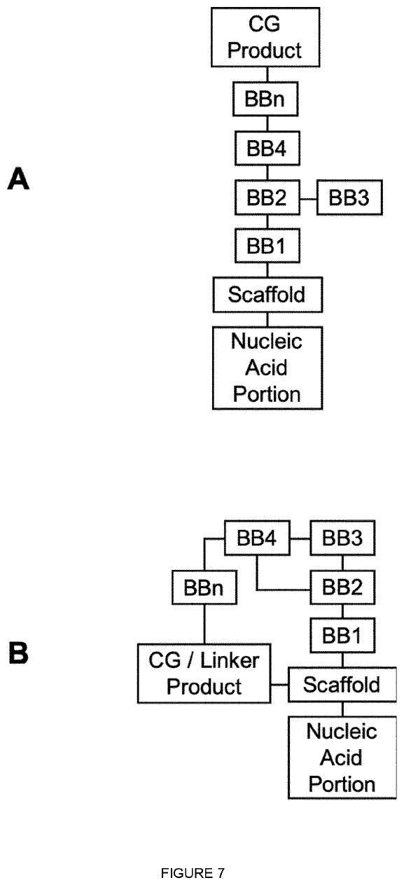

Selectively released self-purified library members may comprise a linear, branched, cyclic, macrocyclic, or polycyclic structure formed from the chemical portion and optionally the scaffold, and one or more cleavage moieties resulting from the reaction of the linker and the cleaving group, or one or more moieties remaining after cleavage of linkers. FIG. 6A shows an example of a self-purified compound with a linear structure. FIG. 6B shows example of a self-purified compound with a cyclic or macrocyclic structure. FIG. 7A shows an example of a self-purified compound with a branched structure. FIG. 7B shows an example of a self-purified compound with bicyclic structure.

After self-purified library members are selectively released from solid support, further reactions may be performed. For example, additional transformations may be performed in solution, or the self-purified compound may be re-captured onto solid support for additional transformations including transformations followed by self-purification reactions. Further transformations may include chemical building block incorporation reactions, crosslinking of chemical building blocks, crosslinking of chemical building blocks to the scaffold, cleavage reactions, ring-opening reactions, and macrocyclization reactions. For example, a bicyclic structure may be formed after a macrocyclic self-purified library member is released from solid support by crosslinking of two building blocks in a CuAAC (copper-catalysed azide-alkyne cycloaddition).

In some embodiments, individual solid support-linked library members may be compartmentalised before the member is released from the solid support. This allows for activity-based assays of self-purified library members. This also allows the released member to be physically separated from the nucleic acid molecule comprising the coding oligonucleotides within the compartment. The presence of the nucleic acid molecule and the released member in the same compartment allows the nucleic acid molecule encoding the released member to be identified and sequenced to determine the chemical building blocks that form the chemical portion of the member. This may be useful for example in activity-based assay systems.

Members may be compartmentalised by isolating each particle of the solid support in a separate compartment. Segregation prevents solid support-bound members of different particles from interacting with each other and allows the coding nucleic acid to remain associated with the released member, even it when it is physically separated from it by the cleavage of the linker. A compartment may comprise an isolated volume or droplet. For example, a volume or droplet of between about 0.5 pL and about 100 nL may be used. However, smaller or larger volumes may also be used. The compartment may comprise the solid support-bound member and suitable reactants, buffers and other reagents to facilitate cleavage of the linker and release of the member from the support. The compartmentalized library may be in any suitable format, for example in an array, microfluidic or micropatterned device, or multiwell dish.

In some embodiments, the coding nucleic acid portion comprising the coding oligonucleotides may be attached to an anchor located between the linker and the solid support, such that the anchor and the coding nucleic acid portion remain linked to the solid support when the linker is cleaved and the member released.

The anchor is a chemical moiety to which the attachment oligonucleotide is attached. Preferably, the same chemical moiety forms the anchor for all of the members of the library.

In some embodiments, a method of producing a DNA encoded library may comprise for each member the steps of;
providing a nascent member comprising a scaffold and an anchor, wherein the scaffold is connected to a linker by the anchor; and the anchor comprises an attachment oligonucleotide and is attached to a solid support,
covalently attaching one or more chemical building blocks to the nascent member to form a chemical portion attached to the scaffold,
covalently attaching one or more coding oligonucleotides to the attachment oligonucleotide to form a nucleic acid portion attached to the anchor,
attaching a cleaving group to the chemical portion or the scaffold,
isolating the nascent member in a compartment,
reacting the linker and the cleaving group, such that the linker is cleaved and the chemical portion attached to the scaffold is released from the solid support in the compartment.

In other embodiments, a method of producing a DNA encoded library may comprise for each member the steps of;
providing a nascent member comprising a scaffold and an anchor, wherein the scaffold is connected to a first linker by the anchor; and the anchor comprises an attachment oligonucleotide and is attached to a solid support,
covalently attaching one or more chemical building blocks to the nascent member to form a chemical portion attached to the scaffold,
covalently attaching one or more coding oligonucleotides to the attachment oligonucleotide to form a nucleic acid portion attached to the anchor,
attaching a second linker to the chemical portion to connect the chemical portion to the solid support,
cleaving the first linker,
isolating the nascent member in a compartment, and
cleaving the second linker, such that the second linker is cleaved and the chemical portion attached to the scaffold is released from the solid support in the compartment.

The chemical portion remains compartmentally associated with the nucleic acid portion, allowing the sequencing to the nucleic portion to identify the compartmentally associated chemical portion.

In other embodiments, the coding nucleic acid portion comprising the coding oligonucleotides may be attached to the solid support, such that the coding nucleic acid portion remains linked to the solid support when the linker is cleaved and the member released. For example, a method of producing a DNA encoded library may comprise for each member the steps of;
providing a nascent member comprising a scaffold connected to a solid support by a linker; and the solid support comprises an attachment oligonucleotide,
covalently attaching one or more chemical building blocks to the nascent member to form a chemical portion attached to the scaffold,
covalently attaching one or more coding oligonucleotides to the attachment oligonucleotide to form a coding nucleic acid portion attached to the solid support,
attaching a cleaving group to the chemical portion or the scaffold,
isolating the nascent member in a compartment,
reacting the linker and the cleaving group, such that the linker is cleaved and the chemical portion attached to the scaffold is released from the solid support in the compartment.

Another method of producing a DNA encoded library may comprise for each member the steps of;
providing a nascent member comprising a scaffold connected to a solid support by a first linker; and the solid support comprises an attachment oligonucleotide,
covalently attaching one or more chemical building blocks to the nascent member to form a chemical portion attached to the scaffold,
covalently attaching one or more coding oligonucleotides to the attachment oligonucleotide to form a coding nucleic acid portion attached to the solid support,
attaching a second linker to the chemical portion to connect the chemical portion to the solid support,
cleaving the first linker, isolating the nascent member in a compartment, and
cleaving the second linker, such that the second linker is cleaved and the chemical portion attached to the scaffold is released from the solid support in the compartment.

In some preferred embodiments of the above aspects of the invention, between 1 and 10¹⁵ copies of the linker may be present on one solid support entity, such as a particle or bead.

Following release from the support, the member or compound may be additionally purified. For example, additional HPLC purification may be performed after self-purification.

A library produced by a method described herein may be screened for members that bind to a target molecule. Library members that bind to the target molecule may be identified and the nucleic acid molecules sequenced to identify the chemical building blocks that form the chemical entities displayed by the identified library members.

The binding of identified library members may be validated in the absence of coding nucleic acid. For example, a method may comprise;
(i) providing a nascent member comprising a labelled scaffold connected to a solid support by a linker,
(ii) covalently attaching the chemical building blocks of the chemical portion of an identified library member to the scaffold to produce a labelled nascent member that comprises said chemical portion,
(iii) attaching a cleaving group to the chemical portion or scaffold,
(iv) reacting the linker with the cleaving group, thereby cleaving the linker and releasing the member from the solid support, and
(vi) determining the binding of the released labelled member to the target molecule.

The binding of the released member to the target molecule may be determined using the label. The scaffold may be labelled with any convenient label, for example a fluorescent label.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

Priority is claimed from EP20203475.7, the disclosure of which is also incorporated herein by reference in its entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Experiments

Materials and Methods

Resin Swelling

Before first use or after storage at −20° C., TentaGel® beads (Rapp Polymere GmbH) were incubated with a suitable solvent for 10 min.

Solid Support Drying and Washing

TentaGel® beads (Rapp Polymere GmbH) were used in a solid-phase synthesis reaction vessel with a frit. Vacuum filtration was used to remove any solvent or solution incubated with the TentaGel® beads. The beads were washed by adding a suitable solvent, and subsequently removing the resulting solvent or solution. Magnetic solid support was separated from solutions by using a magnet.

Solid Support Quantities

The quantity of solid support used in each experiment is given in mmol of the respective surface functionality (loading amount), in mass (g or mg), or as a volume (μL), wherein the volume of beads refers to the volume of bead suspension used at the same concentration as supplied by the manufacturer.

Solid Support Storage

Functionalized TentaGel® beads (Rapp Polymere GmbH) were stored dried at −20° C. Other solid support types were stored as a suspension in a suitable solvent or aqueous solution at 4° C.

Oligonucleotides

Custom oligonucleotides purchased were additionally purified by ethanol precipitation. The redissolved oligonucleotides in mQ were analysed by LCMS and their concentration was measured using a NanoDrop 2000c Spectrophotometer.

Amino-modified single-stranded (ss) DNA
(Sequence 1):
5′ d Amino C6-GGAGCTTCTGAATT 3′
Molecular Weight = 4473.02 Da, ϵ₂₆₀ = 133700 M⁻¹ cm⁻¹
Amino-modified double-stranded (ds) DNA
(Sequence 2):
5′ d Phos-GAGTCA-Spacer 9-Amino C7-Spacer 9-
TGACTCCC 3′
Molecular Weight = 4937.24 Da, ϵ₂₆₀ = 127872 M⁻¹ cm⁻¹
Adaptor (Sequence 3):
3′ CCTCGAAGACTTAAGACACACGAC 5′
Code (Sequence 4):
5′ d Phos-CTGTGTGCTGACAGCTCGAGTCCCATGGCGC 3′
Molecular Weight = 9584.16 Da, ϵ₂₆₀ = 280300 M⁻¹ cm⁻¹

Ethanol Precipitation of Oligonucleotides

Ethanol precipitation was performed by adding 10% (v/v) 5 M NaCl and 3.5 volumes of EtOH. The samples were stored at −20° C. for at least 2 h, and then centrifuged at 4° C. for 1 h at 20800×g. The supernatants were discarded following centrifugation. The precipitate was completely dried in a Christ Alpha RVC Speedvac rotational vacuum concentrator instrument, and then dissolved in mQ (Milli-Q®) water.

Concentration Assessment of Oligonucleotide Solutions

The concentration of oligonucleotide solutions was determined using a NanoDrop 2000c Spectrophotometer instrument by the measurement of UV absorbance at 260 nm. 2 μL of the oligonucleotide solution was used for each measurement. The concentration of the oligonucleotide was calculated from the known absorption coefficient of the DNA sequence and the measured UV absorbance at 260 nm.

Liquid Chromatography-Mass Spectrometry (LCMS)

Mass spectrometry (LCMS) spectra were recorded using a Waters Acquity UPLC and Xevo G2-XS QTof Quadrupole Time of Flight Mass Spectrometer (Waters). An XBridge® Oligonucleotide BEH™ C18, 130 Å, 2.5 μm, 2.1 mm×50 mm column or an XBridge® Oligonucleotide BEH™ C18, 130 Å, 1.7 μm, 2.1 mm×50 mm column was used for LCMS analysis of oligonucleotides. An Acquity UPLC® CSH™ C18, 1.7 μm, 2.1 mm×50 mm column was used for LCMS analysis of small molecules.

General Procedure 1: General Procedure for Amide Coupling of a Carboxylic Acid to Amine-Functionalized Solid Support

The functionalized TentaGel® beads were swollen in DMF (10 mL). The amine-functionalized TentaGel® beads (1 equiv. amine loading amount) were incubated with 66.7 mM respective acid (4 equiv.), 133.3 mM N,N-diisopropylethylamine (DIPEA) (8 equiv.), and 66.7 mM N-[(Dimethylamino)-1H-1,2,3-triazolo-[4,5-b]pyridin-1-ylmethylene]-N-methylmethanaminium hexafluorophosphate N-oxide (HATU) (4 equiv.) in dimethylformamide (6 mL per 0.1 mmol loading amount) at room temperature on a rotational shaker for 2 h. The functionalized TentaGel® beads were washed with dimethylformamide (3×10 mL), dichloromethane (3×10 mL), and then dimethylformamide (3×10 mL).

General Procedure 2: General Procedure for Fmoc Deprotection on Solid Support

The functionalized TentaGel® beads were swollen in DMF (10 mL). The dried functionalized TentaGel® beads were then incubated with 20% (v/v) piperidine in dimethylformamide (6 mL per 0.1 mmol loading amount) at room temperature on a rotational shaker for 30 min. The functionalized TentaGel® beads were washed with dimethylformamide (3×10 mL), dichloromethane (3×10 mL), and then dimethylformamide (3×10 mL).

General Procedure 3: General Procedure for Amide Coupling of an Acid to Amine-Functionalized Magnetic Solid Support

Amine-functionalized solid support (50 μL) was washed with dimethylformamide (200 μL). The amine-functionalized beads were incubated with a solution of 50 mM diisopropylcarbodiimide (DIC), 50 mM ethyl cyano(hydroxyimino)acetate (OxymaPure) and 50 mM respective acid in dimethylformamide (150 μL) on a rotational shaker at room temperature for 4 h. The functionalized beads were washed with dimethylformamide (6×200 μL).

General Procedure 4: General Procedure for Fmoc Deprotection on Magnetic Solid Support

The functionalized beads (50 μL) were washed with dimethylformamide (200 μL). The functionalized beads were incubated with 20% (v/v) piperidine in dimethylformamide (200 μL) at room temperature on a rotational shaker for 1 h. The functionalized beads were washed with dimethylformamide (6×200 μL).

General Procedure 5: Attachment of 5′-Azido Modified Single-Stranded Oligonucleotide (or 5′-Azido Modified Double-Stranded Oligonucleotide) to Solid Support by CuAAC Method 1

The procedure was adapted from MacConnell et al 2015. Alkyne-functionalized TentaGel® beads (20 mg) were swollen in 30 mM triethylammonium acetate pH 8.5 in 50% DMSO in mQ water with 0.034% (v/v) Tween 20 on a rotational shaker at room temperature for 15 min. The dried solid support was reacted with 1-5 nmol 5′-azido modified single-stranded oligonucleotide (synthesized in Example 1) in a solution of 2.6 mM 1-(Phenylmethyl)-N,N-bis[[1-(phenylmethyl)-1H-1,2,3-triazol-4-yl]methyl]-1H-1,2,3-triazole-4-methanamine (TBTA), 14.0 mM sodium ascorbate, and 2.8 mM copper sulfate in 20 mM triethylammonium acetate pH 8.5 in 48% DMSO in mQ water with 0.035% (v/v) Tween 20 (195 μL) on a rotational shaker at 60° C. for 3 h. The solid support was washed with a solution of 10 mM 2,2′-(1,3-Propanediyldiimino)bis[2-(hydroxymethyl)-1,3-propanediol], 100 mM sodium chloride, 10 mM N,N′-1,2-Ethanediylbis[N-(carboxymethyl)glycine] (EDTA) with 1% (v/v) Tween 20 and 1% (w/v) sodium dodecyl sulfate (SDS), pH 7.6 (3×0.6 mL).

General Procedure 6: Attachment of 5′-Azido Modified Single-Stranded Oligonucleotide to Solid Support by CuAAC Method 2

Alkyne-functionalized magnetic solid support (25 μL) with an excess loading capacity was washed with 50% DMSO in mQ water (3×200 μL). The solid support was reacted with 1-5 nmol 5′-azido modified single-stranded oligonucleotide (Synthesized in Example 1) in a mixture of 993 μM 1-(Phenylmethyl)-N,N-bis[[1-(phenylmethyl)-1H-1,2,3-triazol-4-yl]methyl]-1H-1,2,3-triazole-4-methanamine (TBTA), 945 μM copper sulfate (CuSO₄), and 5.672 mM sodium ascorbate (NaAsc), and 100 mM lithium chloride in 42% DMSO in mQ water (90 μL) on a rotational shaker at room temperature for 1 h. The solid support was washed with 50% DMSO in mQ water (3×200 μL).

### Example 1: Preparation of a 5′-Azido Modified Single-Stranded Oligonucleotide

Amino-modified ssDNA: 5′ d Amino C6-GGAGCTTCTGAATT 3′ (Sequence 1)

Acid azide: 3-[2-[2-[2-(2-Azidoethoxy)ethoxy]ethoxy]ethoxy]propanoic acid

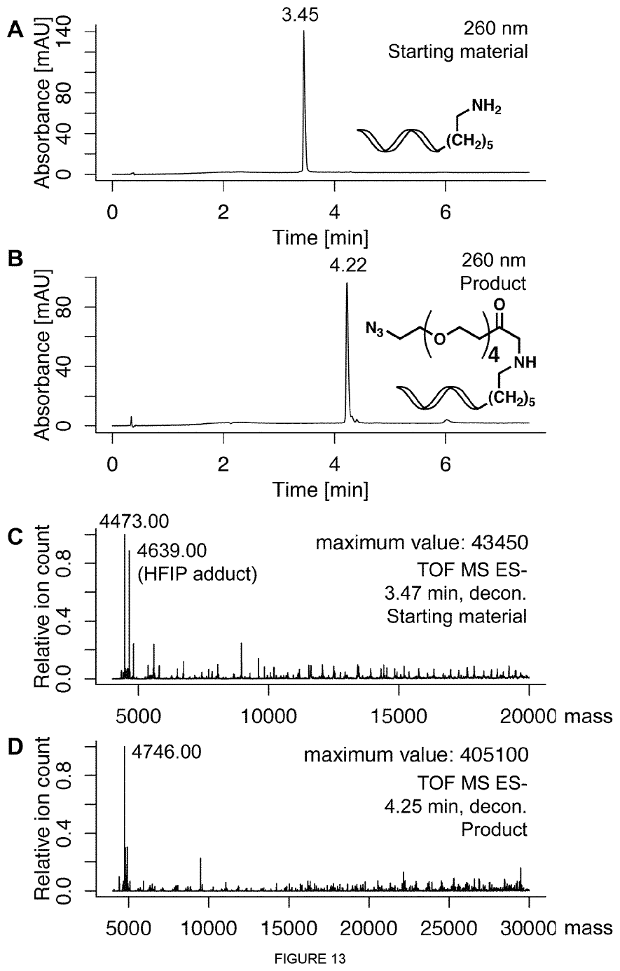

3-[2-[2-[2-(2-Azidoethoxy)ethoxy]ethoxy]ethoxy]propanoic acid (300 μL, 100 mM in DMSO), 1-Hydroxy-2,5-dioxo-3-pyrrolidinesulfonic acid (S—NHS) (145 μL, 100 mM in 33% (v/v) mQ water in DMSO), and N³-(Ethylcarbonimidoyl)-N¹,N¹-dimethyl-1,3-propanediamine (EDC) (145 μL, 100 mM in DMSO) were added to a tube containing 1 mL dimethylsulfoxide (DMSO) and were incubated on a rotational shaker at 37° C. for 30 min. In the meantime, 250 nmol amino-modified ssDNA in 504 μL mQ water was incubated with 350 μL 250 mM borate buffer pH 9.5 on a rotational shaker at 37° C. for 30 min. The activation solution containing S—NHS, EDC, and 3-[2-[2-[2-(2-Azidoethoxy)ethoxy]ethoxy]ethoxy]propanoic acid was combined with the DNA in borate buffer and left to react for 45 min at 37° C. on a rotational shaker. The reaction was monitored by LCMS. The DNA was precipitated by addition of 20% (v/v) of 5 M NaCl followed by 3.5 volumes of absolute ethanol. The sample was stored at −20° C. for 18 h, and was then centrifuged at 4° C. and 4000×g. The supernatant was discarded, and the precipitate was completely dried in a Speedvac rotational vacuum concentrator instrument. The dried precipitate was dissolved in 1 mL 100 mM TEAA pH 7.0. The crude was purified by RP-HPLC using a Waters XBridge® BEH C18 OBD™ Prep Column (130 Å, 5 μm, 10 mm×150 mm) and a gradient of buffer 1, 100 mM TEAA pH 7.0 buffer, and buffer 2, 100 mM TEAA pH 7.0 in 80% acetonitrile in mQ water. The collected fractions were combined and concentrated, and the oligonucleotide was precipitated by addition of 20% (v/v) of 5 M NaCl followed by 3.5 volumes of absolute ethanol. The sample was stored at −20° C. for 2 h, and was then centrifuged at 4° C. and 4000×g. The supernatant was discarded, and the precipitate was completely dried in a Speedvac rotational vacuum concentrator instrument. The dried precipitate was dissolved in 500 μL mQ water. Concentration assessment by measurement of UV Absorbance at 260 nm on a NanoDrop 2000c spectrophotometer showed a 61% yield. The product was analyzed by LCMS (FIG. 13). In (FIG. 13B), the chromatogram measuring 260 nm absorbance is shown for the product, 5′-azido modified single-stranded oligonucleotide. The desired product is the major product. (FIG. 13D) shows the deconvoluted (decon.) mass spectrum for the product peak. The mass corresponding to the desired product is observed. For comparison, LCMS spectra for the starting material are shown in (FIG. 13A) and (FIG. 13C). (FIG. 13A), the chromatogram measuring 260 nm absorbance is shown for the starting material oligonucleotide (Sequence 1). The deconvoluted (decon.) mass spectrum is shown in (FIG. 13C) for the starting material oligonucleotide (Sequence 1).

### Example 2: Nascent Library Member Preparation (Linker+Cleaving Group Approach)

NH2-functionalized 10 μm TentaGel® beads (Rapp Polymere GmbH) (385 mg, 0.1 mmol loading amount) were added to a solid-phase synthesis reaction vessel with a frit. The TentaGel® beads were swollen with dimethylformamide (6 mL) at room temperature for 10 min on a rotational shaker, and then washed with dimethylformamide (3×10 mL).

Step 2.1 Coupling to Mono-Tert-Butyl Succinate

The functionalized TentaGel® beads were coupled to mono-tert-butyl succinate following general procedure 1.

Step 2.2 Capping

The functionalized TentaGel® beads were swollen with dichloromethane (6 mL) at room temperature for 10 min on a rotational shaker, and then washed with dichloromethane (3×10 mL). The functionalized TentaGel® beads were incubated with a mixture of dichloromethane (6 mL), acetic anhydride (2 mL), and N,N-diisopropylethylamine (DIPEA) (2 mL) at room temperature for 1 h. The functionalized TentaGel® beads were washed with DCM (3×10 mL).

Step 2.3 Tert-Butyl Deprotection

To the functionalized TentaGel® beads was added a mixture of 50% (v/v) trifluoroacetic acid in dichloromethane (5 mL). After 10 min on the rotational shaker at room temperature, the solution was removed. Then, a fresh mixture of 50% (v/v) trifluoroacetic acid in dichloromethane (5 mL) was incubated with the TentaGel® beads for 20 min at room temperature on the rotational shaker. The resin was washed with dichloromethane (3×10 mL), and then dimethylformamide (3×10 mL).

Step 2.4 Coupling to N-Boc-ethylenediamine

The functionalized TentaGel® beads were swollen in DMF (10 mL). The dried functionalized TentaGel® beads were then incubated with N-Boc-ethylenediamine (63 μL, 0.4 mmol, 4 equiv.), N,N-diisopropylethylamine (DIPEA) (136 μL, 0.8 mmol, 8 equiv.), and N-RDimethylamino)-1H-1,2,3-triazolo-[4,5-b]pyridin-1-ylmethyleneFN-methylmethanaminium hexafluorophosphate N-oxide (HATU) (152 mg, 0.4 mmol, 4 equiv.) in dimethylformamide (6 mL) at room temperature on a rotational shaker for 2 h. The functionalized TentaGel® beads were washed with dimethylformamide (3×10 mL), dichloromethane (3×10 mL), and then dimethylformamide (3×10 mL).

Step 2.5 Boc Deprotection

To the functionalized TentaGel® beads was added a mixture of 50% (v/v) trifluoroacetic acid in dichloromethane (5 mL). After 10 min on the rotational shaker at room temperature, the solution was removed. Then, a fresh mixture of 50% (v/v) trifluoroacetic acid in dichloromethane (5 mL) was incubated with the TentaGel® beads for 20 min at room temperature on the rotational shaker. The resin was washed with dichloromethane (3×10 mL), and then dimethylformamide (3×10 mL).

Step 2.6 Coupling to 6-(Fmoc-amino)hexanoic Acid

The functionalized TentaGel® beads were swollen in DMF (10 mL). The functionalized TentaGel® beads were then coupled to 6-(Fmoc-amino)hexanoic acid following general procedure 1.

Step 2.7 Fmoc Deprotection

The functionalized TentaGel® beads were Fmoc deprotected according to general procedure 2.

Step 2.8 Coupling to 3-[[(9H-Fluoren-9-ylmethoxy)carbonyl]amino]-4-(methylamino)benzoic Acid

The functionalized TentaGel® beads were reacted with 3-[[(9H-Fluoren-9-ylmethoxy)carbonyl]amino]-4-(methylamino)benzoic acid (Fmoc-MeDbz-OH) following general procedure 1.

Step 2.9 Fmoc Deprotection

The functionalized TentaGel® beads were Fmoc deprotected according to general procedure 2.

Step 2.10 Coupling to 6-(Fmoc-amino)hexanoic Acid

The functionalized TentaGel® beads were reacted with 6-(Fmoc-amino)hexanoic acid following general procedure 1.

Step 2.11 Fmoc Deprotection

The functionalized TentaGel® beads were Fmoc deprotected according to general procedure 2.

Step 2.12 Coupling to (2S)-2-[[(9H-Fluoren-9-ylmethoxy)carbony]amino]-4-pentynoic Acid

The functionalized TentaGel® beads were coupled to (2S)-2-[[(9H-Fluoren-9-ylmethoxy)carbonyl]amino]-4-pentynoic acid (Fmoc-Pra-OH) following general procedure 1.

Step 2.13 Fmoc Deprotection

The functionalized TentaGel® beads were Fmoc deprotected according to general procedure 2.

Step 2.14 Oligonucleotide Attachment

The compound synthesized in steps 2.1-2.13 is solid support with a MeDbz linker connecting to the scaffold, which comprises a site for building block attachment at the amine functional group, and an alkyne for nucleic acid attachment. 5 nmol 5′-azido modified single-stranded oligonucleotide (synthesized in Example 1) was attached to functionalized TentaGel (steps 2.1-2.13, 20 mg) following general procedure 5.

Nascent Library Member Cleavage for LCMS Analysis

Step 2.15 MeDbz Linker Activation Step 1—Incubation with p-Nitrophenyl Chloroformate

The functionalized TentaGel® beads (20 mg) with oligonucleotide attached were incubated with 100 mM p-nitrophenyl chloroformate in dichloromethane (600 μL) on a rotational shaker at room temperature for 30 min. The resin was washed with dichloromethane (3×600 μL).

Step 2.16 MeDbz Linker Activation Step 2—Incubation with N,N-diisopropylethylamine (DIPEA)

The functionalized TentaGel® beads (20 mg) with oligonucleotide attached were incubated with 8.7% (v/v) N,N-diisopropylethylamine (DIPEA) in dichloromethane (600 μL) on a rotational shaker at room temperature for 30 min. The resin was washed with dichloromethane (2×600 μL).

Step 2.17 MeDbz Cleavage by Cysteamine

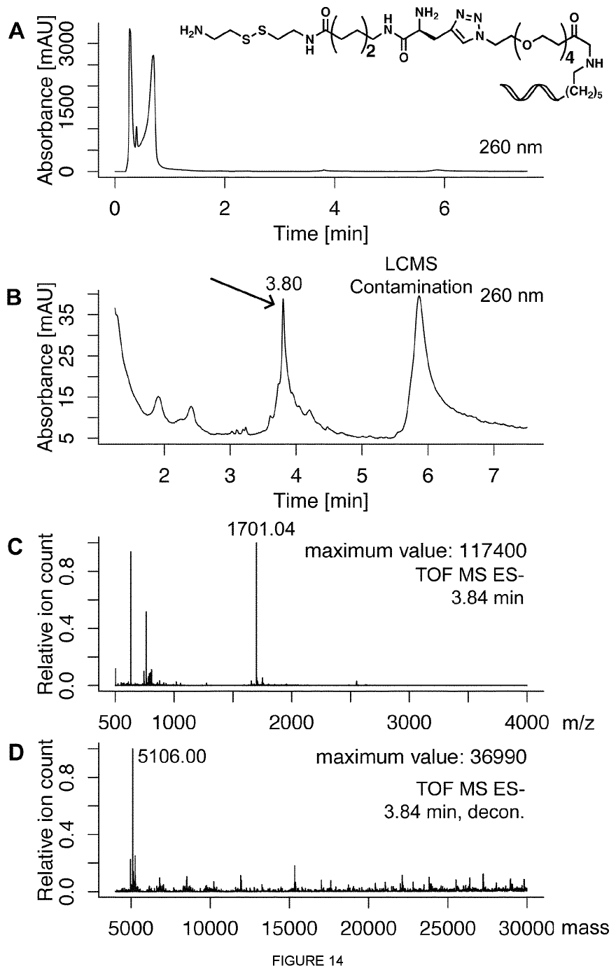

The functionalized TentaGel® beads (20 mg) with oligonucleotide attached were incubated with excess cysteamine in 50% (v/v) DMSO in mQ water with 0.01% (w/v) sodium dodecyl sulfate (SDS) (150 μL) on a rotational shaker at 60° C. for 1 h. The resulting solution was separated from the beads by centrifugation and was collected. The collected solution was analyzed by LCMS (FIG. 14). FIG. 14 shows analytical LCMS data for the cleavage of the nascent library member. (FIG. 14A), and (FIG. 14B) show chromatograms measuring 260 nm absorbance. (FIG. 14C), and (FIG. 14D) show the mass spectrum of the product peak at 3.80 min, and the deconvoluted mass spectrum, respectively. These LCMS spectra show that the desired nascent library member was obtained. The large peak observed at a later retention time is an LCMS impurity seen in all LCMS measurements on that machine at the time.

### Example 3: Synthesis of a Self-Elution Model Compound Off-DNA

The functionalized solid support prepared in steps 2.1-2.13 was used as a starting material for the synthesis of the self-elution model compound off-DNA. The functionalized TentaGel® beads were swollen with dimethylformamide (6 mL) at room temperature for 10 min on a rotational shaker, and then washed with dimethylformamide (3×10 mL).

Step 3.1 Resin Splitting

The functionalized TentaGel® beads prepared in steps 2.1-2.13 were split into two portions of 0.05 mmol. The synthesis was continued on a 0.05 mmol scale with one of the two portions.

Step 3.2 Coupling to 6-(Fmoc-amino)hexanoic Acid

The functionalized TentaGel® beads were reacted with 6-(Fmoc-amino)hexanoic acid following general procedure 1.

Step 3.3 Fmoc Deprotection

The functionalized TentaGel® beads were Fmoc deprotected according to general procedure 2.

Step 3.4 Coupling to (2S)-2-([[(9H-Fluoren-9-yl)methoxy]carbonyl]amino)-3-(naphthalen-1-yl)propanoic Acid

The functionalized TentaGel® beads were reacted with (2S)-2-([[(9H-Fluoren-9-yl)methoxy]carbonyl]amino)-3-(naphthalen-1-yl)propanoic acid (Fmoc-1-NaI—OH) following general procedure 1.

Step 3.5 Resin Splitting

The functionalized TentaGel® beads were split into two portions of 0.025 mmol. The synthesis was continued on a 0.025 mmol scale with one of the two portions.

Step 3.6 Fmoc Deprotection

The functionalized TentaGel® beads were Fmoc deprotected according to general procedure 2.

Step 3.7 Coupling to (RS)-Lipoic Acid

The functionalized TentaGel® beads were reacted with (RS)-Lipoic acid following general procedure 1.

### Example 4: Self-Elution of a Model Nucleic Acid Encoded Library Member

Step 4.1 Oligonucleotide Attachment

5 nmol 5′-azido modified single-stranded oligonucleotide was attached to the self-elution model compound synthesized in Example 3 (20 mg) following general procedure 5.

Step 4.2 MeDbz Linker Activation Step 1—Incubation with p-Nitrophenyl Chloroformate

The functionalized TentaGel® beads (20 mg) with oligonucleotide attached were incubated with 100 mM p-nitrophenyl chloroformate in dichloromethane (600 μL) on a rotational shaker at room temperature for 30 min. The resin was washed with dichloromethane (3×600 μL).

Step 4.3 MeDbz Linker Activation Step 2—Incubation with N,N-diisopropylethylamine (DIPEA)

The functionalized beads were incubated with 8.7% (v/v) N,N-diisopropylethylamine (DIPEA) in dichloromethane (600 μL) on a rotational shaker at room temperature for 30 min. The resin was washed with dichloromethane (3×600 μL).

Step 4.4 Cleaving Group Deprotection

The functionalized beads were incubated in a solution of 100 mM Tris(2-carboxyethyl)phosphine hydrochloride (TCEP) in 6% (v/v) triethylamine, 53% dimethylsulfoxide (DMSO) in mQ water with 0.01% (w/v) sodium dodecyl sulfate (SDS), pH 8-9 (150 μL) on a rotational shaker at 60° C. for 1 h. The beads were dried by centrifugation.

Step 4.5 Self-Elution of Model Nucleic Acid Encoded Library Member

The functionalized beads were incubated with a solution of 10% (v/v) N,N-diisopropylethylamine (DIPEA) in 80% acetonitrile in mQ water with 0.01% (w/v) sodium dodecyl sulfate (SDS) (150 μL) on a rotational shaker at 60° C. for 1 h. The resulting solution was separated from the beads by centrifugation and was collected. The collected solution was concentrated using a Speedvac rotational vacuum concentrator instrument.

Step 4.6 Ethanol Precipitation of Self-Eluted Model Nucleic Acid Encoded Library Member

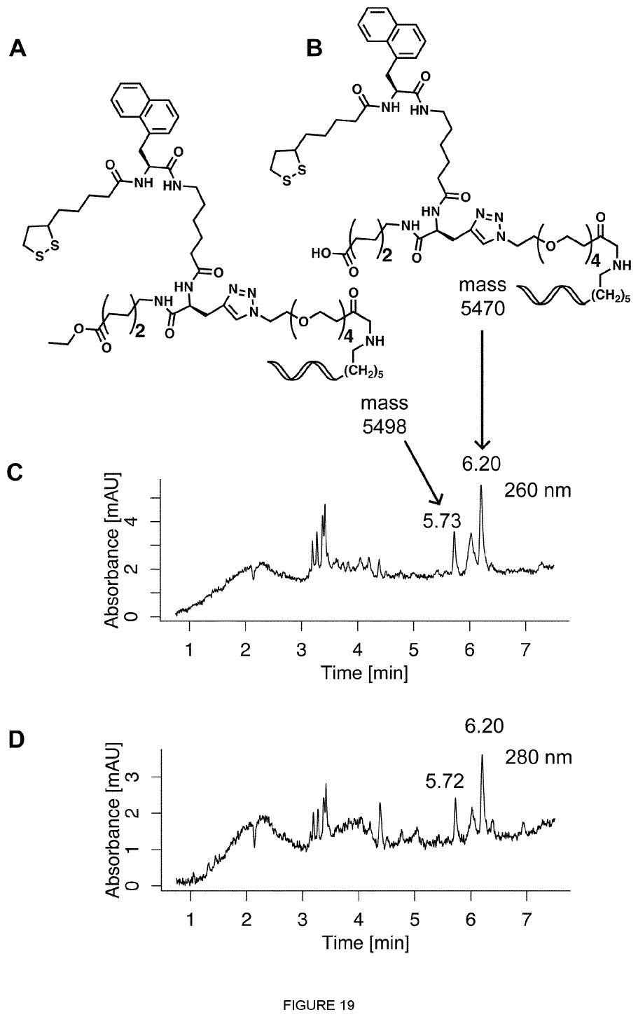

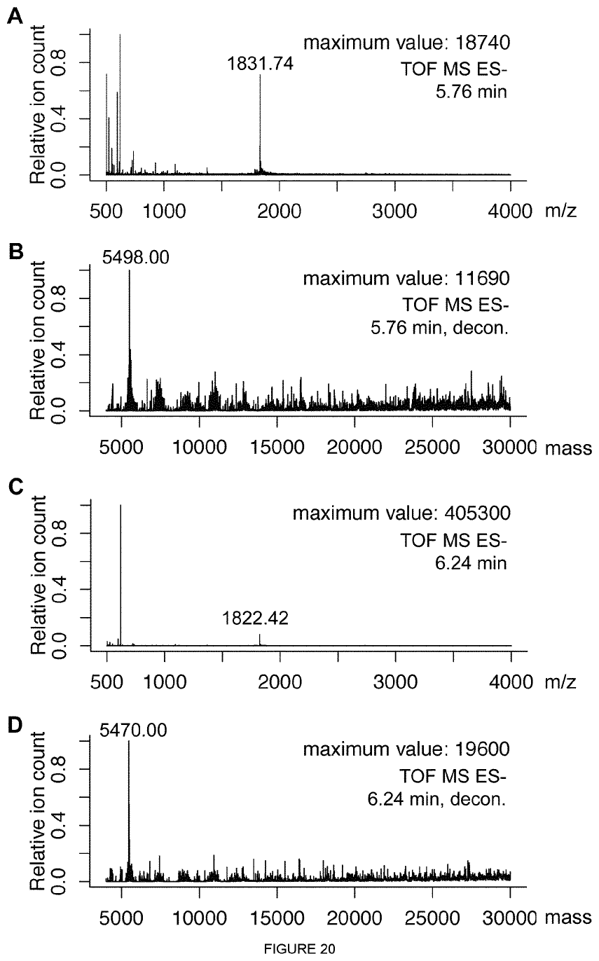

The residue obtained after step 4.5 was resuspended in 50% (v/v) dimethylsulfoxide (DMSO) in mQ water (150 μL). The sample was filtered and 10 μL were of the sample was used for LCMS analysis. 125 μL of the sample was used to ethanol precipitate the self-eluted model nucleic acid encoded library member by adding 10% (v/v) of 5 M sodium chloride (12.5 μL), 10% (v/v) of 2.5 M sodium acetate buffer pH 4.79 (12.5 μL), followed by 3.5 volumes of absolute ethanol (525 μL). The sample was stored at −20° C. for 18 h, and was then centrifuged at 4° C. and 20800×g for 1 h. The supernatant was discarded, and the precipitate was completely dried in a Speedvac rotational vacuum concentrator instrument. The dried precipitate was dissolved in mQ water (100 μL). LCMS analysis showed the mass of the self-eluted model nucleic acid encoded library member (FIG. 19, FIG. 20). The chemical structures (FIG. 19A), and (FIG. 19B) were observed in the chromatograms at 260 nm (FIG. 19C), and 280 nm (FIG. 19D) absorbance. (FIG. 19B) is the linear self-eluted product formed by the hydrolysis of the cyclic thioester intermediate. (FIG. 19A) is the linear self-eluted product formed by the ring opening of the cyclic thioester intermediate by ethanol, which was added for the precipitation step. The compounds observed may be formed by the oxidation of the thiol groups to form an intramolecular disulfide bond. (FIG. 20A), and (FIG. 20B) show the mass spectrum and the deconvoluted mass spectrum, respectively, for the self-eluted library member compound illustrated in (FIG. 19A). (FIG. 20C), and (FIG. 20D) show the mass spectrum and the deconvoluted mass spectrum, respectively, for the self-eluted library member compound illustrated in (FIG. 19B). These data show that self-elution was achieved.

### Example 5: Self-Elution of a Model Nucleic Acid Encoded Library Member

Step 5.1 Oligonucleotide Attachment

5 nmol of 5′-azido modified single-stranded oligonucleotide was attached to the self-elution model compound synthesized in Example 3 (20 mg) following general procedure 5.

Step 5.1 MeDbz Linker Activation Step 1—Incubation with p-nitrophenyl Chloroformate

The functionalized TentaGel® beads (20 mg) with oligonucleotide attached were incubated with 100 mM p-nitrophenyl chloroformate in dichloromethane (600 μL) on a rotational shaker at room temperature for 30 min. The resin was washed with dichloromethane (3×600 μL).

Step 5.2 MeDbz Linker Activation Step 2—Incubation with N,N-diisopropylethylamine (DIPEA)

The functionalized beads were incubated with 8.7% (v/v) N,N-diisopropylethylamine (DIPEA) in dichloromethane (600 μL) on a rotational shaker at room temperature for 30-40 min. The resin was washed with dichloromethane (3×600 μL).

Step 5.3 Cleaving Group Deprotection

The functionalized beads were incubated in a solution of 100 mM Tris(2-carboxyethyl)phosphine hydrochloride (TCEP) in 6% (v/v) triethylamine, 53% dimethylsulfoxide (DMSO) in mQ water with 0.01% (w/v) sodium dodecyl sulfate (SDS), pH 8-9, on a rotational shaker at 60° C. for 1 h. The beads were dried by centrifugation.

Step 5.4 Self-Elution of Model Nucleic Acid Encoded Library Member

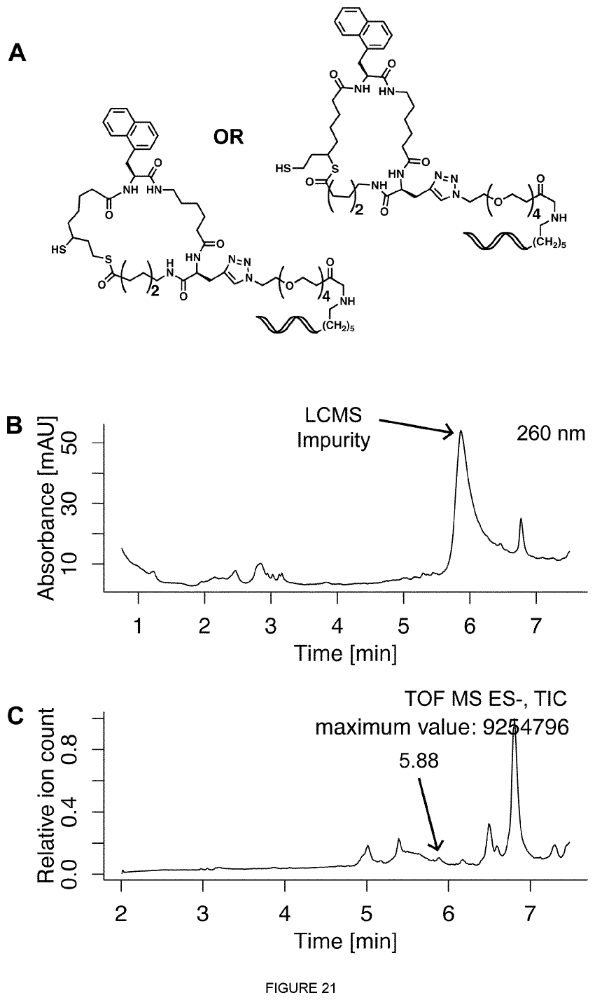

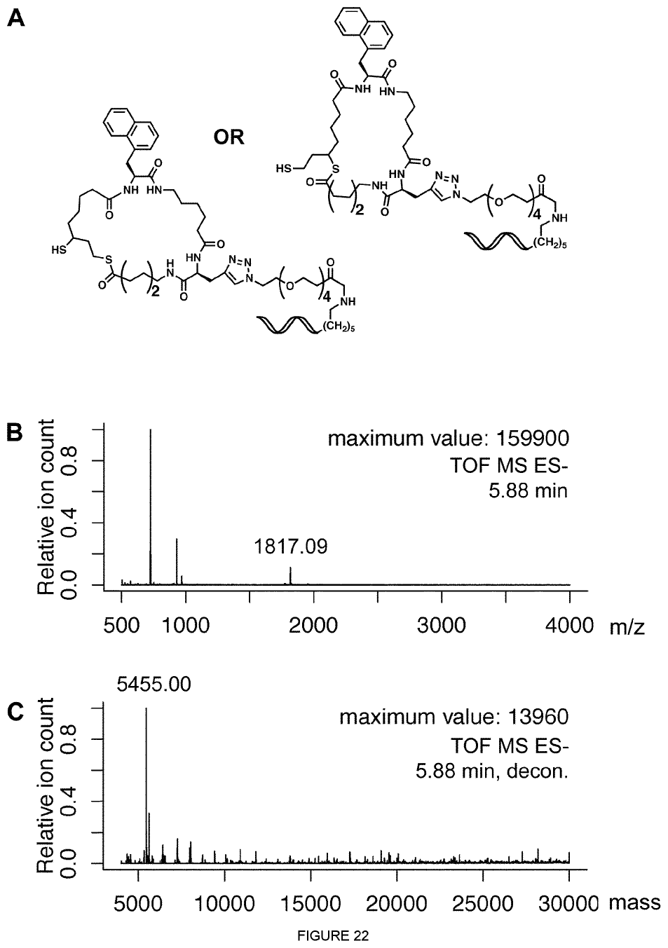

The functionalized beads were incubated with a solution of 10% (v/v) N,N-diisopropylethylamine (DIPEA) in dichloromethane (150 μL) on a rotational shaker at 60° C. for 1 h. The beads were then dried by centrifugation. The beads were washed with a solution of 1 mM sodium carbonate (Na₂CO₃) in 49% DMSO in mQ water, with 0.01% SDS, pH 9, and the resulting solution collected by centrifugation was analyzed by LCMS (FIG. 21, FIG. 22). (FIG. 21A) shows the structures of the two possible cyclic self-eluted nucleic acid encoded library members. (FIG. 21B) is the chromatogram at 260 nm, and (FIG. 21C) is the TIC. The peak with the mass corresponding to the cyclic self-eluted library members is observed at 5.88 min. (FIG. 22B), and (FIG. 22C) are the mass spectrum for the peak for the self-eluted nucleic acid library member, and the corresponding deconvoluted mass spectrum, respectively. The mass of the desired cyclic self-eluted nucleic acid encoded library member was observed, which shows that self-elution was achieved.

### Example 6: Self-Purification of Model Nucleic Acid Encoded Library Member

Step 6.1 Dbz Linker Derivative Synthesis

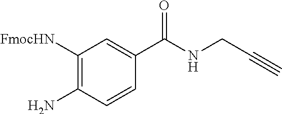

4-Amino-3-[[(9H-fluoren-9-ylmethoxy)carbonyl]amino]benzoic acid (Fmoc-Dbz-OH) (200 mg, 0.534 mmol, 1.0 equiv) was dissolved in 10 mL dimethylformamide (DMF) in a round bottom flask. Propargylamine (68.3 μL, 1.068 mmol, 2.0 equiv), N,N-diisopropylethylamine (DIPEA) (372 μL, 2.137 mmol, 4.0 equiv.), and N-[(Dimethylamino)-1H-1,2,3-triazolo-[4,5-b]pyridin-1-ylmethylene]-N-methylmethanaminium hexafluorophosphate N-oxide (HATU) (407 mg, 1.068 mmol, 2.0 equiv.) were added and the solution was stirred at room temperature for 2 h. The reaction mixture was quenched with water. The aqueous component was extracted with ethyl acetate. The combined organic components were concentrated under reduced pressure. The crude product was purified by flash column chromatography (70% (v/v) ethyl acetate in hexane) to yield a white solid.

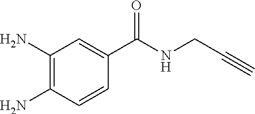

The above product was stirred in 20% (v/v) diethylamine in THF (10 mL) at room temperature for 5 h. The reaction mixture was concentrated under reduced pressure. The crude reaction product was used for attachment to solid support.

Preparation of a Nascent Library Member

Step 6.2 Coupling to N²-[(9H-Fluoren-9-ylmethoxy)carbonyl]-N⁶-[(4-methylphenyl)diphenylmethyl]-L-lysine (Fmoc-Lys(Mtt)-OH)

Amine-functionalized solid support (functionalized ProMag® 1, Bangs Labarotories, Inc.) (50 μL) was coupled to N²-[(9H-Fluoren-9-ylmethoxy)carbonyl]-N⁶-[(4-methylphenyl)diphenylmethyl]-L-lysine (Fmoc-Lys(Mtt)-OH) following general procedure 3.

Step 6.3 Fmoc Deprotection

The functionalized beads (50 μL) were Fmoc deprotected following general procedure 4.

Step 6.4 Coupling to 4-(Hydroxymethyl)benzoic acid (HMBA)

Amine-functionalized solid support (50 μL) was coupled to 4-(Hydroxymethyl)benzoic acid (HMBA) following general procedure 3.

Step 6.5 Coupling to (2S)-2-[[(9H-Fluoren-9-ylmethoxy)carbonyl]amino]-4-pentynoic acid (Fmoc-Pra-OH)

Alcohol-functionalized solid support (50 μL) was washed with dimethylformamide (200 μL). The functionalized beads were incubated with a solution of 100 mM diisopropylcarbodiimide (DIC), 5.76 mM DMAP and 80 mM (2S)-2-[[(9H-Fluoren-9-ylmethoxy)carbonyl]amino]-4-pentynoic acid (Fmoc-Pra-OH) in dimethylformamide (150 μL) on a rotational shaker at 4° C. for 4 h. The functionalized beads were washed with dimethylformamide (6×200 μL).

Step 6.6 Fmoc Deprotection

The functionalized beads (50 μL) were Fmoc deprotected following general procedure 4.

Step 6.7 Coupling to 1-(1,1-Dimethylethyl) Butanedioate Building Block

Amine-functionalized solid support (50 μL) was coupled to 1-(1,1-Dimethylethyl) butanedioate following general procedure 3.

Step 6.8 Mtt Deprotection and tBu Deprotection

The functionalized beads (50 μL) were incubated with 50% (v/v) trifluoroacetic acid in dichloromethane (600 μL) at room temperature on a rotational shaker for 3 min (step repeated 3×). The functionalized beads were washed with 10% (v/v) N,N-diisopropylethylamine (DIPEA) in dimethylformamide (3×200 μL), and then with dimethylformamide (3×200 μL).

Step 6.9 Oligonucleotide Attachment to Solid Support

5 nmol 5′-azido modified single-stranded oligonucleotide (synthesized in Example 1) was attached to the alkyne-functionalized beads (50 μL) following general procedure 6, using double the stated volumes.

Step 6.10 Small Molecule Alkyne Quenching

The functionalized beads (50 μL) after DNA attachment were subjected to CuAAC conditions with benzyl azide to quench any unreacted alkyne functional groups. The functionalized solid support (50 μL) was washed with dimethylsulfoxide (DMSO) (3×200 μL). The solid support was incubated in a mixture of 50 mM benzyl azide, 993 μM 1-(Phenylmethyl)-N,N-bis[[1-(phenylmethyl)-1H-1,2,3-triazol-4-yl]methyl]-1H-1,2,3-triazole-4-methanamine (TBTA), 945 μM copper sulfate (CuSO₄), and 5.672 mM sodium ascorbate (NaAsc) 89% dimethylsulfoxide (DMSO) in mQ water (360 μL) on a rotational shaker at room temperature for 1 h. The solid support was washed with DMSO (3×200 μL). 5 μL of the functionalized solid support was kept for analysis.

Installation of Linker 2

Step 6.11 Coupling to 5-azidopentanoic acid (Reaction On-DNA on Solid Support)

50 mg/mL stocks of N³-(Ethylcarbonimidoyl)-N¹,N¹-dimethyl-1,3-propanediamine (EDC) and of 1-Hydroxy-2,5-dioxo-3-pyrrolidinesulfonic acid (S—NHS) were prepared in 100 mM 2-(N-morpholino)ethanesulfonic acid (MES) buffer pH 4.5. 40 μL of each stock was mixed with 120 μL of 450 mM 5-azidopentanoic acid in dimethylformamide (DMF) to result in a final volume of 200 μL. The solution was incubated for 15 min at room temperature. The solution was then added to the functionalized beads (45 μL) and the reaction was left on a rotational shaker at room temperature for 2 h. The functionalized beads were washed with dimethylformamide (DMF) (3×200 μL).

Step 6.12 Coupling to Reverse Dbz Linker 2 (Reaction On-DNA on Solid Support)

5 μL of the functionalized solid support was kept for analysis. The rest of the functionalized solid support (40 μL) was washed with dimethylformamide (DMF) (200 μL). The functionalized beads were incubated with a solution of 360 mM amine (reverse Dbz linker prepared in step 6.1), 360 mM HATU, and 1.50 M DIPEA in dimethylformamide (DMF) (100 μL) at 40° C. on a rotational shaker at room temperature for 1 h. The functionalized beads were washed with dimethylformamide (DMF) (3×200 μL).

Step 6.13 Oligonucleotide Cleavage of 10 μL Beads from Solid Support for LCMS Analysis

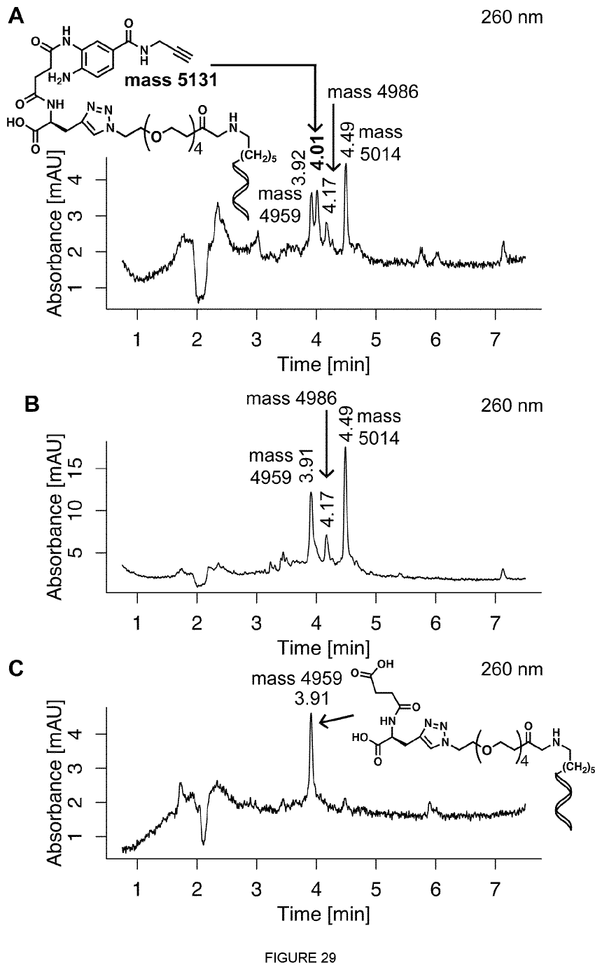

A portion of the functionalized solid support (10 μL) was incubated with 100 mM lithium hydroxide (LiOH) in 25% dimethylsulfoxide (DMSO) in mQ water (60 μL) for 1 h at 40° C. on a rotational shaker. The sample was filtered and analyzed by LCMS (FIG. 29A). The LCMS spectrum shows that cleavage of the HMBA linker yields the desired intermediate, shown at 4.01 min in chromatogram (FIG. 29A). The chemical structure of the desired intermediate is shown (FIG. 29A). Additionally, the sample analyzed comprises undesired intermediate products. These may, for example, be compounds which did not undergo a previous amide coupling step in the synthesis.

Step 6.14 Cyclisation by CuAAC

The remaining functionalized solid support (30 μL) was washed with dimethylsulfoxide (DMSO) (3×200 μL). The solid support was incubated in a mixture of 993 μM 1-(Phenylmethyl)-N,N-bis[[1-(phenylmethyl)-1H-1,2,3-triazol-4-yl]methyl]-1H-1,2,3-triazole-4-methanamine (TBTA), 945 μM copper sulfate (CuSO₄), and 5.672 mM sodium ascorbate (NaAsc) in 89% DMSO in mQ water (270 μL) on a rotational shaker at room temperature for 1 h. The solid support was washed with DMSO (3×200 μL).

Self-Purification

Step 6.15 Cleavage of Linker 1

For the functionalized solid support (30 μL), the HMBA linker 1 was cleaved by 100 mM lithium hydroxide (LiOH) in 25% DMSO in mQ water (60 μL) at 40° C. for 1.5 h on a rotational shaker. LCMS analysis of this solution showed undesired products cleaved from solid support during this step (FIG. 29B). Comparison of chromatogram (FIG. 29B) with chromatogram (FIG. 29A) shows that all compounds are cleaved from solid support in step 6.15, except the desired intermediate in formed in step 6.12, since this compound comprises an alkyne and has undergone a CuAAC reaction before the cleavage in step 6.15. This illustrates that the CuAAC reaction has been successful and only undesired products are released from solid support in this step.

Step 6.16 Cleavage of Linker 2 (Terminal Linker)

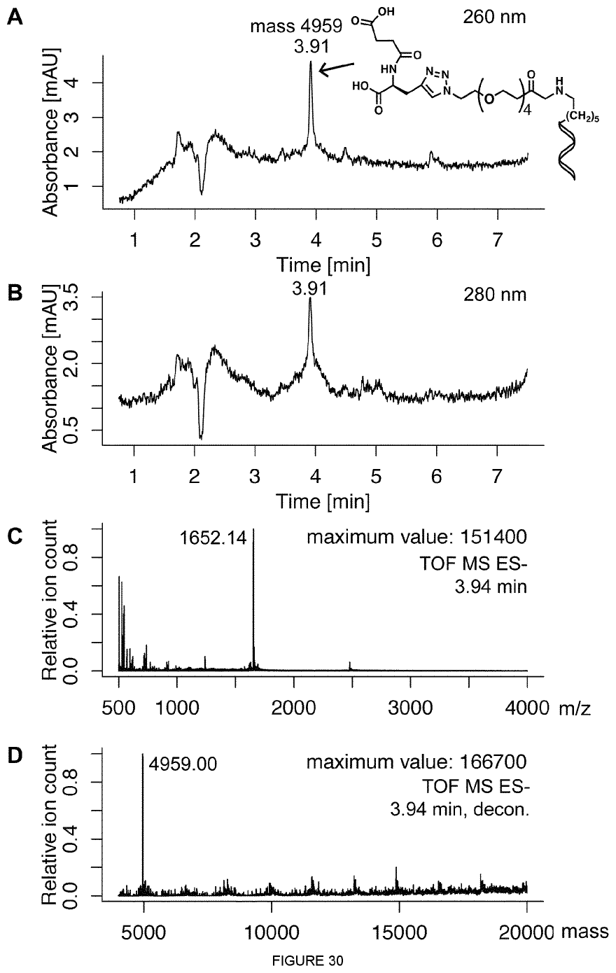

The Dbz linker 2 on the functionalized solid support (30 μL) was activated by incubation with 36 mM isopentyl nitrite in mQ water (200 μL) for 1.5 h at room temperature on a rotational shaker. The solid support was washed with mQ water (2×200 μL). The activated linker was cleaved in 100 mM lithium hydroxide (LiOH) in 25% DMSO in mQ water (60 μL). LCMS analysis showed the desired self-purified nucleic acid encoded library member (FIG. 29C, FIG. 30). FIG. 29C shows a chromatogram of the sample obtained after cleaving the second linker, Dbz. The chromatogram shows the self-purified nucleic acid encoded library member. The structure of the desired, self-purified nucleic acid encoded library member is shown (FIG. 29C). FIG. 30A shows the chromatogram for 260 nm absorbance. FIG. 30B shows the chromatogram for 280 nm absorbance. FIG. 30C shows the mass spectrum of the product peak at 3.94 min. FIG. 30D shows the deconvoluted mass spectrum of the product peak at 3.94 min. The mass corresponding to the desired self-purified model nucleic acid encoded library member is observed. The desired self-purified model nucleic acid encoded library member is the major product obtained in step 6.16.

### Example 7: Self-Purification of Model Nucleic Acid Encoded Library Member

Step 7.1 Coupling to N²-[(9H-Fluoren-9-ylmethoxy)carbonyl]-N⁶-[(4-methylphenyl)diphenylmethyl]-L-lysine (Fmoc-Lys(Mtt)-OH)

Amine-functionalized solid support (functionalized ProMag® 1, Bangs Labarotories, Inc.) (50 μL) was coupled to N²-[(9H-Fluoren-9-ylmethoxy)carbonyl]-N⁶-[(4-methylphenyl)diphenylmethyl]-L-lysine (Fmoc-Lys(Mtt)-OH) following general procedure 3.

Step 7.2 Fmoc Deprotection

The functionalized beads (50 μL) were Fmoc deprotected following general procedure 4.

Step 7.3 Coupling to 4-(Hydroxymethyl)benzoic Acid (HMBA)

Amine-functionalized solid support (50 μL) was coupled to 4-(Hydroxymethyl)benzoic acid (HMBA) following general procedure 3.

Step 7.4 Coupling to (2S)-2-[[(9H-Fluoren-9-ylmethoxy)carbonyl]amino]-4-pentynoic Acid (Fmoc-Pra-OH)

Alcohol-functionalized solid support (50 μL) was washed with dimethylformamide (DMF) (200 μL). The functionalized beads were incubated with a solution of 100 mM diisopropylcarbodiimide (DIC), 5.76 mM N,N-dimethyl-4-pyridinamine (DMAP) and 80 mM (2S)-2-[[(9H-Fluoren-9-ylmethoxy)carbonyl]amino]-4-pentynoic acid (Fmoc-Pra-OH) in dimethylformamide (150 μL) on a rotational shaker at 4° C. for 4 h. The functionalized beads were washed with dimethylformamide (DMF) (6×200 μL).

Step 7.5 Fmoc Deprotection

The functionalized beads (50 μL) were Fmoc deprotected following general procedure 4.

Step 7.6 Coupling to Fmoc-Dbz-OH

Amine-functionalized solid support (50 μL) was coupled to 4-Amino-3-[[(9H-fluoren-9-ylmethoxy)carbonyl]amino]benzoic acid (Fmoc-Dbz-OH) following general procedure 3.

Step 7.7 Mtt Deprotection

The functionalized beads (50 μL) were incubated with 50% (v/v) trifluoroacetic acid in dichloromethane (600 μL) at room temperature on a rotational shaker for 3 min (step repeated 3×). The functionalized beads were washed with 10% (v/v) N,N-diisopropylethylamine (DIPEA) in dimethylformamide (3×200 μL), and then with dimethylformamide (3×200 μL).

Step 7.8 Oligonucleotide Attachment to Solid Support

5 nmol 5′-azido modified single-stranded oligonucleotide (synthesized in Example 1) was attached to the alkyne-functionalized beads (50 μL) following general procedure 6, using double the stated volumes.

Step 7.9 Small Molecule Alkyne Quenching

The functionalized beads (50 μL) after DNA attachment were subjected to CuAAC conditions with benzyl azide to quench any unreacted alkyne functional groups. The functionalized solid support (50 μL) was washed with DMSO (3×200 μL). The solid support was incubated in a mixture of 50 mM benzyl azide, 993 μM 1-(Phenylmethyl)-N,N-bis[[1-(phenylmethyl)-1H-1,2,3-triazol-4-yl]methyl]-1H-1,2,3-triazole-4-methanamine (TBTA), 945 μM copper sulfate (CuSO4), and 5.672 mM sodium ascorbate (NaAsc) 89% DMSO in mQ water (360 μL) on a rotational shaker at room temperature for 1 h. The solid support was washed with DMSO (3×200 μL). 5 μL of the functionalized solid support was kept for analysis.

Step 7.10 Coupling to 5-azidopentanoic Acid (Reaction On-DNA on Solid Support)

50 mg/mL stocks of N³-(Ethylcarbonimidoyl)-N¹,N¹-dimethyl-1,3-propanediamine (EDC) and of 1-Hydroxy-2,5-dioxo-3-pyrrolidinesulfonic acid (S—NHS) were prepared in 100 mM 2-(N-morpholino)ethanesulfonic acid (MES) buffer pH 4.5. 40 μL of each stock was mixed with 120 μL of 450 mM 5-azidopentanoic acid in dimethylformamide to result in a final volume of 200 μL. The solution was incubated for 15 min at room temperature. The solution was then added to the functionalized beads (45 μL) and the reaction was left on a rotational shaker at room temperature for 2 h. The functionalized beads were washed with dimethylformamide (3×200 μL). 5 μL of the functionalized solid support was kept for analysis.

Step 7.11 Fmoc Deprotection

The functionalized beads (40 μL) were Fmoc deprotected following general procedure 4, using 80% of the stated volumes.

Step 7.12 Coupling to 5-hexynoic Acid

The functionalized solid support (40 μL) was washed with DMSO (200 μL). The solid support was incubated in a solution of 5 mM 3-Hydroxy-3H-1,2,3-triazolo[4,5-b]pyridine (HOAt), 500 mM 5-hexynoic acid, and 50 mM N³-(Ethylcarbonimidoyl)-N¹,N¹-dimethyl-1,3-propanediamine (EDC) in DMSO (500 μL) at room temperature for 2 h on a rotational shaker. The solid support was washed with DMSO (3×200 μL). 10 μL of the functionalized solid support was kept for analysis.

Step 7.13 Cyclisation by CuAAC

Alkyne- and azide-functionalized solid support (30 μL) was washed with DMSO (3×200 μL).

The solid support was incubated in a mixture of 993 μM 1-(Phenylmethyl)-N,N-bis[[1-(phenylmethyl)-1H-1,2,3-triazol-4-yl]methyl]-1H-1,2,3-triazole-4-methanamine (TBTA), 945 μM copper sulfate (CuSO₄), and 5.672 mM sodium ascorbate (NaAsc) 89% DMSO in mQ water (360 μL) on a rotational shaker at room temperature for 1 h. The solid support was washed with DMSO (3×200 μL).

Self-Purification

Step 7.14 Cleavage of Linker 1

For the functionalized solid support (30 μL), the HMBA linker 1 was cleaved by 100 mM lithium hydroxide (LiOH) in 25% DMSO in mQ water (60 μL) at 40° C. for 1.5 h on a rotational shaker.

Step 7.15 Cleavage of Linker 2 (Terminal Linker)

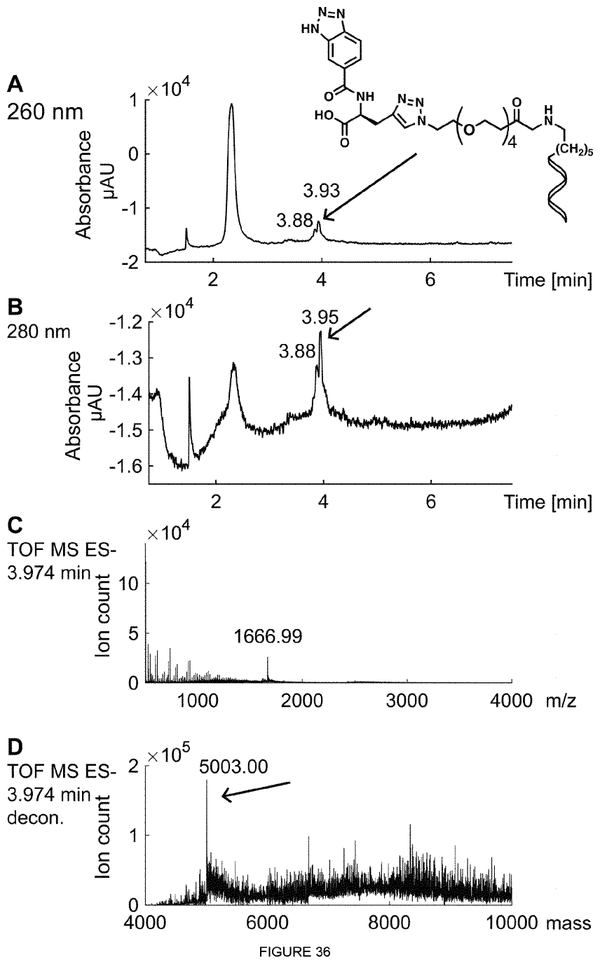

For the functionalized solid support (30 μL), the Dbz linker 2 was activated by incubation with 36 mM isopentyl nitrite in mQ water (200 μL) for 2 h at room temperature on a rotational shaker. The solid support was washed with mQ water (2×200 μL). The activated linker was cleaved in 100 mM lithium hydroxide (LiOH) in 25% DMSO in mQ water (60 μL). The cleavage solution was analyzed by LCMS (FIG. 36). FIG. 36A shows the chromatogram for 260 nm absorbance. FIG. 36B shows the chromatogram for 280 nm absorbance. FIG. 36C shows the mass spectrum of the product peak at 3.97 min. FIG. 36D shows the deconvoluted mass spectrum of the product peak at 3.97 min. The mass corresponding to the desired self-purified model nucleic acid encoded library member is observed. This example additionally shows that the Dbz linker has been activated prior to cleavage.

### Example 8: DNA Ligation on Solid Support

Adaptor (Sequence 3):
3′-CCTCGAAGACTTAAGACACACGAC-5′
Code (Sequence 4):
5′ d Phos-CTGTGTGCTGACAGCTCGAGTCCCATGGCGC 3′
Molecular Weight = 9584.16 Da, ϵ₂₆₀ = 280300 M⁻¹ cm⁻¹

Step 8.1 Coupling to Ethylenediamine

Carboxylic acid functionalized magnetic solid support (ProMag® 1, Bangs Labarotories, Inc.) (25 μL) was washed with DMSO (1×1 mL) and dimethylformamide (DMF) (2×1 mL). The functionalized beads were incubated with 360 mM HATU, 360 mM ethylenediamine and 1.1 M DIPEA in DMF (25 μL, 2×30 min). The functionalized beads were washed with dimethylformamide (3×200 μL).

Step 8.2 Coupling to 4-(Hydroxymethyl)benzoic Acid (HMBA)

Amine-functionalized solid support (25 μL) was coupled to 4-(Hydroxymethyl)benzoic acid (HMBA) following general procedure 3, using half of the respective stated volumes.

Step 8.3 Coupling to N²-[(9H-Fluoren-9-ylmethoxy)carbonyl]-N⁶-[(4-methylphenyl)diphenylmethyl]-L-lysine (Fmoc-Lys(Mtt)-OH)

Amine-functionalized solid support (25 μL) was coupled to N²-[(9H-Fluoren-9-ylmethoxy)carbonyl]-N⁶-[(4-methylphenyl)diphenylmethyl]-L-lysine (Fmoc-Lys(Mtt)-OH) following general procedure 3, using half of the respective stated volumes.

Step 8.4 Fmoc Deprotection

The functionalized beads (25 μL) were Fmoc deprotected following general procedure 4, using half of the respective stated volumes.

Step 8.5 Coupling to 1-(9H-Fluoren-9-ylmethyl) 5,8,11,14-tetraoxa-2-azaheptadecanedioate (Fmoc-PEG₄-OH)

Amine-functionalized solid support (25 μL) was coupled to 1-(9H-Fluoren-9-ylmethyl) 5,8,11,14-tetraoxa-2-azaheptadecanedioate (Fmoc-PEG₄-OH) following general procedure 3, using half of the respective stated volumes.

Step 8.6 Fmoc Deprotection

The functionalized beads (25 μL) were Fmoc deprotected following general procedure 4, using half of the respective stated volumes.

Step 8.7 Coupling to 5-hexynoic Acid

Amine-functionalized solid support (25 μL) was coupled to 5-hexynoic acid following general procedure 3, using half of the respective stated volumes.

Step 8.8 Mtt Deprotection

The functionalized beads (25 μL) were incubated with 50% (v/v) trifluoroacetic acid in dichloromethane (300 μL) at room temperature on a rotational shaker for 3 min (step repeated 3×). The functionalized beads were washed with 10% (v/v) N,N-diisopropylethylamine (DIPEA) in dimethylformamide (3×200 μL), and then with dimethylformamide (3×200 μL).

Step 8.9 Oligonucleotide Attachment to Solid Support

1 nmol 5′-azido modified single-stranded oligonucleotide (synthesized in Example 1) was attached to the functionalized solid support (25 μL) following general procedure 6, however without any lithium chloride.

Step 8.10 Ligation on Solid Support

The procedure for ligation was adapted from Pengpumkiat et al 2016. The functionalized solid support was washed with a solution of 10 mM Tris, 1 mM EDTA, 2 M NaCl, and 0.05% (v/v) Tween 20, pH 7.4 (1×200 μL). The functionalized solid support was then washed with mQ water (2×200 μL). To the functionalized solid support (25 μL) was added 1.9 nmol adaptor (Sequence 3) in 90 μL mQ water, and 10 μL of a solution of 100 mM Tris, 500 mM NaCl, 10 mM EDTA, pH 7.4. The mixture was heated to 95° C. for 10 min. The sample was left to cool to room temperature for 1 h. 1.5 nmol code (Sequence 4), 17 μL mQ water, 2 μL of 10× T4 ligase buffer (500 mM Tris-HCl, 100 mM MgCl₂, 10 mM ATP, 100 mM DTT, pH 7.5, New England Biolabs) and 600 U (1 μL) T4 DNA ligase (New England Biolabs) were added to the mixture, and the ligation was performed at room temperature for 18 h. The functionalized solid support was washed with TE Buffer (10 mM Tris, 1 mM EDTA and 0.05% (v/v) Tween 20, pH 7.4).

Step 8.11 Oligonucleotide Cleavage from Solid Support for LCMS Analysis

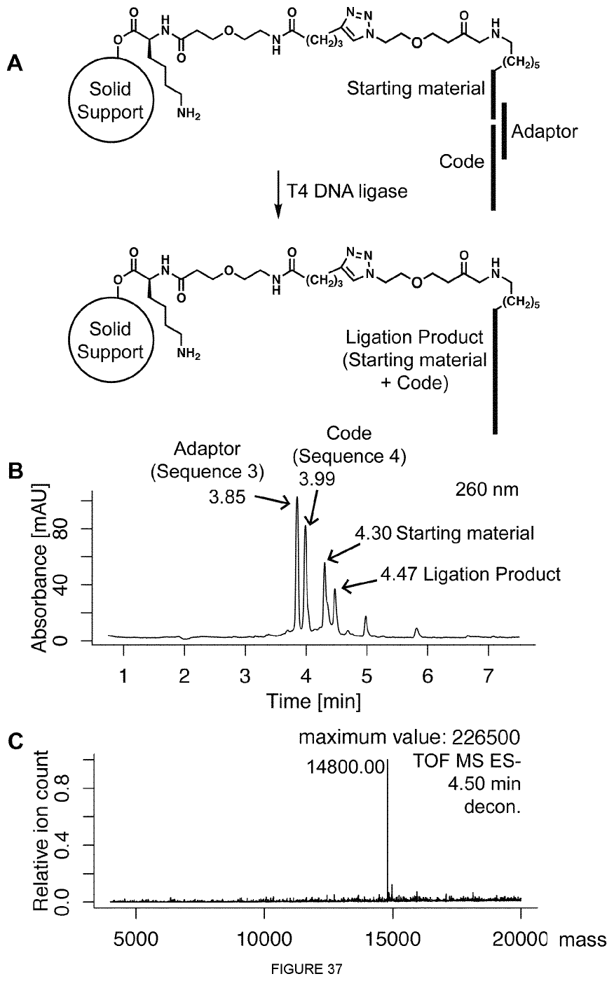

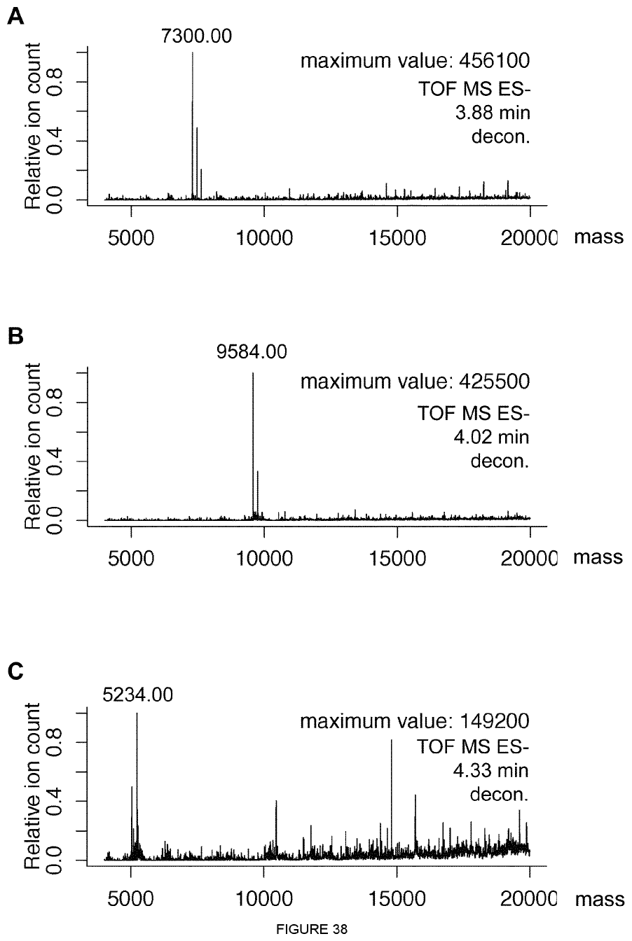

The functionalized solid support (25 μL) was incubated with 100 mM lithium hydroxide (LiOH) in 25% DMSO in mQ water (60 μL) for 1 h at 40° C. on a rotational shaker. The sample was filtered and analysed by LCMS (FIG. 37, FIG. 38). (FIG. 37A) shows a schematic representation for DNA ligation on solid support. The code is ligated to the oligonucleotide attached to the solid support by using an adaptor oligonucleotide and T4 DNA ligase. The LCMS chromatogram for 260 nm absorbance (FIG. 37B) was obtained by cleaving the ester linker after the ligation conditions (step 8.11). The ligation product can be seen at 4.47 min. Remaining adaptor, code, and unligated starting material was additionally observed. FIG. 37C shows the deconvoluted mass spectrum for the ligation product peak. The mass for the desired ligation product was observed. FIG. 38 shows deconvoluted mass spectra for (FIG. 38A) the adaptor oligonucleotide, (FIG. 38B) the code, and (FIG. 38C) the starting material peaks for the LCMS chromatogram shown in FIG. 37B.

### Example 9: Reaction On-DNA On-Solid Support

Carboxylic acid-functionalized solid support (functionalized ProMag® 1, Bangs Laboratories, Inc.) was used.

Step 9.1 Coupling to N-Boc-ethanolamine

Carboxylic acid functionalized magnetic solid support (50 μL) with was washed with DMSO (1×1 mL) and dimethylformamide (2×1 mL). The functionalized beads were incubated with 360 mM HATU, 360 mM N-Boc-ethanolamine and 1.1 M DIPEA in DMF (25 μL, 2×30 min). The functionalized beads were washed with dimethylformamide (DMF) (3×200 μL).

Step 9.2 Boc Deprotection

The functionalized beads (50 μL) were incubated with 50% (v/v) trifluoroacetic acid in dichloromethane (600 μL) at room temperature on a rotational shaker for 5 min. The beads were then again incubated with a fresh solution of 50% (v/v) trifluoroacetic acid in dichloromethane (600 μL) at room temperature on a rotational shaker for 15 min. The functionalized beads were washed with 1×PBS pH 7.4 (3×600 μL), and then with dimethylformamide (3×600 μL).

Step 9.3 Coupling to (2S)-2-[[(9H-Fluoren-9-ylmethoxy)carbonyl]amino]-4-pentynoic acid (Fmoc-Pra-OH)

Amine-functionalized solid support (50 μL) was coupled to (2S)-2-[[(9H-Fluoren-9-ylmethoxy)carbonyl]amino]-4-pentynoic acid (Fmoc-Pra-OH) following general procedure 3.

Step 9.4 Fmoc Deprotection

The functionalized beads (50 μL) were Fmoc deprotected following general procedure 4.

Step 9.5 Oligonucleotide Attachment to Solid Support

2 nmol 5′-azido modified single-stranded oligonucleotide (synthesized in Example 1) was attached to the alkyne-functionalized beads (50 μL) following general procedure 6, using double the respective stated volumes.

Step 9.6 Oligonucleotide Cleavage of 25 μL Beads from Solid Support for LCMS Analysis

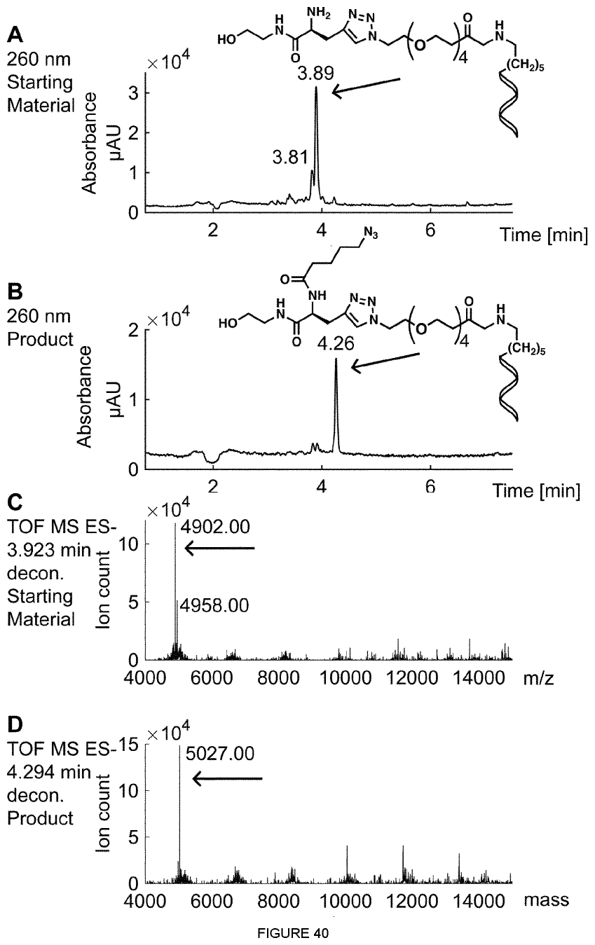

Half of the functionalized solid support (25 μL) was incubated with 100 mM lithium hydroxide (LiOH) in 25% DMSO in mQ water (60 μL) for 1 h at 40° C. on a rotational shaker. The sample was filtered and analysed by LCMS. FIG. 40A shows the chromatogram at 260 nm, and the chemical structure of the starting material. The major peak observed in FIG. 40A corresponds to the desired starting material. The deconvoluted mass spectrum at the retention time of the major peak in the chromatogram (FIG. 40A) is shown in FIG. 40C. The mass corresponding to the desired starting material is observed.

Step 9.7 Coupling to 5-azidopentanoic Acid (Reaction On-DNA on Solid Support)

The remaining half of the functionalized solid support (25 μL) prepared in step 9.5 was used. 50 mg/mL stocks of N³-(ethylcarbonimidoyl)-N¹,N¹-dimethyl-1,3-propanediamine (EDC) and of 1-Hydroxy-2,5-dioxo-3-pyrrolidinesulfonic acid (S—NHS) were prepared in 100 mM 2-(N-morpholino) ethanesulfonic acid (MES) buffer pH 4.5. 20 μL of each stock was mixed with 60 μL of 450 mM 5-azidopentanoic acid in dimethylformamide (DMF) to result in a final volume of 100 μL. The solution was incubated for 15 min at room temperature. The solution was then added to the functionalized beads (25 μL) and the reaction was left on a rotational shaker at room temperature for 2 h. The functionalized beads were washed with dimethylformamide (DMF) (3×200 μL).

Step 9.8 Oligonucleotide Cleavage from Solid Support for LCMS Analysis

The functionalized solid support (25 μL) prepared in step 9.7 was incubated with 100 mM lithium hydroxide (LiOH) in 25% dimethylsulfoxide (DMSO) in mQ water (60 μL) for 1 h at 40° C. on a rotational shaker. The sample was filtered and analysed by LCMS. FIG. 40B shows the chromatogram at 260 nm, and the chemical structure of the desired product. The major peak observed in FIG. 40B corresponds to the desired product. The deconvoluted mass spectrum at the retention time of the major product in the chromatogram (FIG. 40B) is shown in FIG. 40D. The mass corresponding to the desired product is observed. This example shows that chemical transformations can be performed on solid support on DNA with a high conversion.

### REFERENCES

(1) Agouridas, V et al Native Chemical Ligation and Extended Methods: Mechanisms, Catalysis, Scope, and Limitations. Chemical Reviews. American Chemical Society Jun. 26, 2019. https://doi.org/10.1021/acs.chemrev.8b00712.
(2) Li, Y. et al. Bioorthogonal Ligation and Cleavage by Reactions of Chloroquinoxalines with Ortho-Dithiophenols. Angew. Chemie Int. Ed. 2020, 59 (9), 3671-3677. https://doi.org/10.1002/anie.201913620.
(3) Gless B. H., et al. Direct Peptide Cyclization and One-Pot Modification Using the MeDbz Linker. J. Org. Chem. 2018, 83 (17), 10525-10534. https://doi.org/10.1021/acs.joc.8b01237.
(4) Fan, L.; Davie, C. P. Zirconium(IV)-Catalyzed Ring Opening of on-DNA Epoxides in Water. ChemBioChem 2017, 18 (9), 843-847. https://doi.org/10.1002/cbic.201600563.
(5) Serra, G.; et al. On-Resin Synthesis of Cyclic Peptides: Via Tandem N-to-S Acyl Migration and Intramolecular Thiol Additive-Free Native Chemical Ligation. Chem. Commun. 2020, 56 (6), 956-959. https://doi.org/10.1039/c9cc07783a.
(6) Kondasinghe, T. D. et al Direct Palladium-Mediated on-Resin Disulfide Formation from Allocam Protected Peptides. Org. Biomol. Chem. 2017, 15 (14), 2914-2918. https://doi.org/10.1039/c7ob00536a.
(7) Mende, F.; Seitz, O. Solid-Phase Synthesis of Peptide Thioesters with Self-Purification. Angew. Chemie Int. Ed. 2007, 46 (24), 4577-4580. https://doi.org/10.1002/anie.200700356.
(8) Mende, F.; Beisswenger, M.; Seitz, O. Automated Fmoc-Based Solid Phase Synthesis of Peptide Thioesters with Self-Purification Effect and Application in the Construction of Immobilized SH3 Domains. J. Am. Chem. Soc. 2010, 132 (32), 11110-11118. https://doi.org/10.1021/ja101732a.
(9) Liu, H.; Li, X. Serine/Threonine Ligation: Origin, Mechanistic Aspects, and Applications. Acc. Chem. Res. 2018, 51 (7), 1643-1655. https://doi.org/10.1021/acs.accounts.8b00151.
(10) Clark, M. A. et al. A. Design, Synthesis and Selection of DNA-Encoded Small-Molecule Libraries. Nat. Chem. Biol. 2009, 5 (9), 647-654. https://doi.org/10.1038/nchembio.211.
(11) Rhodes, C. A.; Pei, D. Bicyclic Peptides as Next-Generation Therapeutics. Chem. A Eur. J. 2017, 23 (52), 12690-12703. https://doi.org/10.1002/chem.201702117.
(12) Malone, M. L, Paegel, B. M., What is a “DNA-Compatible” Reaction?, ACS Comb. Sci. 2016, 18 (4), 182-187. https://doi.org/10.1021/acscombsci.5b00198.
(13) Decurtins, W., Wichert, M., Franzini, R. et al. Automated screening for small organic ligands using DNA-encoded chemical libraries. Nat Protoc 11, 764-780 (2016). https://doi.org/10.1038/nprot.2016.039
(14) Selvaraj, A.; Chen, H.-T.; Ya, A.; Huang, T.; Kao, C.-L. Expedient On-Resin Modification of a Peptide C-Terminus through a Benzotriazole Linker. Chem. Sci., 2018, 9, 345-349. https://doi.org/10.1039/C7SC03229C.
(15) Andrew B. MacConnell, Patrick J. McEnaney, Valerie J. Cavett, and Brian M. Paegel, ACS Combinatorial Science 2015 17 (9), 518-534, DOI: 10.1021/acscombsci.5b00106
(16) Pengpumkiat S, Koesdjojo M, Rowley E R, Mockler T C, Remcho V T (2016). PLOS ONE 11(3): e0149774. https://doi.org/10.1371/journal.pone.0149774
(17) Scott, P. J. H. Linker Strategies in Solid-Phase Organic Synthesis. Link. Strateg. Solid-Phase Org. Synth. 1-677 (2009). https://doi/10.1002/9780470749043.
(18) Hermanson, G. T. Bioconjugate Techniques: Third Edition. Bioconjugate Tech. Third Ed. 1-1146 (2013) https://doi.org/10.1016/C2009-0-64240-9.
(19) Leriche, G., Chisholm, L. & Wagner, A. Cleavable linkers in chemical biology. Bioorg. Med. Chem. 20, 571-582 (2012). https://doi.org/10.1016/j.bmc.2011.07.048
(20) Chaudhuri, R. G. & Paria, S. Core/Shell Nanoparticles: Classes, Properties, Synthesis Mechanisms, Characterization, and Applications. Chem. Rev. 112, 2373-2433 (2011). https://doi.org/10.1021/cr100449n (21) Wu, W., He, Q. & Jiang, C. Magnetic Iron Oxide Nanoparticles: Synthesis and Surface Functionalization Strategies. Nanoscale Res. Lett. 2008 311 3, 397-415 (2008). https://doi.org/10.1007/s11671-008-9174-9
(22) Pon, R. T. Solid-Phase Supports for Oligonucleotide Synthesis. Curr. Protoc. Nucleic Acid Chem. 00, (2000). https://doi.org/10.1002/0471142700.nc0301s00
(23) Wang, W., Khojasteh, S. C. & Su, D. Biosynthetic Strategies for Macrocyclic Peptides. Mol. 26, 3338 (2021). https://doi.org/10.3390/molecules26113338
(24) Zhang, R. Y., Thapa, P., Espiritu, M. J., Menon, V. & Bingham, J. P. From nature to creation: Going around in circles, the art of peptide cyclization. Bioorg. Med. Chem. 26, 1135-1150 (2018). https://doi.org/10.1016/j.bmc.2017.11.017

1. A method for producing a nucleic acid encoded compound which includes the steps of;
providing a nascent compound that comprises a scaffold connected to a solid support by a linker,
covalently attaching one or more chemical building blocks to the nascent compound to form the chemical portion attached to the scaffold,
covalently attaching coding oligonucleotides encoding the one or more chemical building blocks to the nascent compound to form a coding nucleic acid portion attached to the scaffold,
attaching a cleaving group to the chemical portion, coding nucleic acid portion, or scaffold,
reacting the linker and the cleaving group, such that the linker is cleaved and the compound released from the solid support.

2. A method for producing a nucleic acid encoded chemical library comprising, for each library member, the steps of;
providing a nascent member that comprises a scaffold connected to a solid support by a linker,
covalently attaching one or more chemical building blocks to the nascent member to form the chemical portion attached to the scaffold,
covalently attaching coding oligonucleotides encoding the one or more chemical building blocks to the nascent member to form a coding nucleic acid portion attached to the scaffold,
attaching a cleaving group to the chemical portion, coding nucleic acid portion, or scaffold,
reacting the linker and the cleaving group, such that the linker is cleaved and the member is released from the solid support.

3. A method according to claim 1 or claim 2 wherein the scaffold of the nascent member is connected to the solid support by a first linker and a second linker and the method further comprises;
attaching a first cleaving group and a second cleaving group to the nascent compound chemical portion or scaffold,
reacting the first linker and the first cleaving group, such that the first linker is cleaved,
reacting the second linker and the second cleaving group, such that the second linker is cleaved and the compound or member is released from the solid support.

4. A method according to any one of claims 1 to 3 wherein the cleaving group is attached to the chemical portion or the scaffold.

5. A method according to claim 4 wherein the cleaving group is covalently attached to the terminal chemical building block of the chemical portion.

6. A method according to claim 4 wherein the cleaving group is attached to the chemical portion or scaffold by a method comprising;
attaching an anchor oligonucleotide to the chemical portion or scaffold,
providing an auxiliary oligonucleotide covalently attached to a cleaving group,
hybridizing the auxiliary oligonucleotide with the anchor oligonucleotide, and
reacting the linker and the cleaving group, such that the linker is cleaved.

7. A method according to any one of claims 1 to 3 wherein the cleaving group is attached to the coding nucleic acid portion.

8. A method according to claim 7 wherein the cleaving group is attached to the coding nucleic acid portion by a method comprising;
providing an auxiliary oligonucleotide covalently attached to a cleaving group,
hybridizing the auxiliary oligonucleotide covalently attached to the cleaving group to the coding nucleic acid portion, and
reacting the linker and the cleaving group, such that the linker is cleaved.

9. A method according to any one of claims 1 to 8 wherein the linker is activated before reaction with the cleaving group.

10. A method according to any one of claims 1 to 9 wherein the cleaving group is activated before reaction with the linker.

11. A method for producing a nucleic acid encoded compound comprising;
providing a nascent compound that comprises a scaffold connected to a solid support by a first linker,
covalently attaching one or more chemical building blocks to the nascent compound to form a chemical portion attached to the scaffold,
covalently attaching coding oligonucleotides encoding the one or more chemical building blocks to the nascent compound to form a coding nucleic acid portion attached to the scaffold,
connecting the chemical portion of the compound to the solid support with a second linker,
cleaving the first linker,
cleaving the second linker, such that the compound is released from the solid support.

12. A method for producing a nucleic acid encoded chemical library comprising, for each library member, the steps of;
providing a nascent member that comprises a scaffold connected to a solid support by a first linker,
covalently attaching one or more chemical building blocks to the nascent member to form a chemical portion attached to the scaffold,
covalently attaching coding oligonucleotides encoding the one or more chemical building blocks to the nascent member to form a coding nucleic acid portion attached to the scaffold,
connecting the chemical portion of the member to the solid support with a second linker,
cleaving the first linker,
cleaving the second linker, such that the member is released from the solid support.

13. A method according to claim 11 and claim 12 wherein the second linker connects the solid support with the scaffold or the nucleic acid portion.

14. A method according to any one of claims 2 to 13 wherein the nucleic acid encoded chemical library is produced by a method comprising the steps of;
splitting nascent members or nucleic acid encoded library intermediates into separate compartments,
attaching one or more chemical building blocks,
attaching one or more coding oligonucleotides encoding the chemical building blocks,
and pooling members or intermediates from separate compartments into one or more compartments.

15. A method according to claim 14 wherein the chemical building blocks and coding oligonucleotides attached are different for members or intermediates in different compartments.

16. A method comprising repeating the steps of claim 14 or 15 one or more times.

17. A method according to any one of claims 11 to 16 where the first and second linkers are orthogonally cleavable.

18. A method according to any one of claims 11 to 17 wherein the first and/or the second linker are activated before cleavage.

19. A method according to any one of claims 1 to 18 wherein the chemical building blocks are attached sequentially to the nascent member to form the chemical portion.

20. A method according to any one of claims 1 to 19 wherein comprising covalently attaching a first chemical building block to the scaffold.

21. A method according to claim 20 wherein the scaffold comprises a capture group and the method comprises reacting a first chemical building block with the capture group to covalently attach the first chemical building block to the scaffold.

22. A method according to claim 21 wherein the first chemical building block comprises a proximal binding group and the method comprises reacting the proximal binding group with the capture group of the scaffold to covalently attach the first chemical building block to the scaffold.

23. A method according to claim 21 or claim 22 wherein the capture group is protected and the method comprises deprotecting the capture group of the scaffold before attachment of the first chemical building block of the chemical portion.

24. A method according to any one of claims 21 to 23 wherein the method comprises capping unreacted capture groups that are not covalently attached to the first chemical building block.

25. A method according to any one of claims 20 to 24 comprising covalently attaching a second chemical building block to the first chemical building block to form a chemical portion having the second chemical building block at an end position.

26. A method according to claim 25 wherein the first chemical building block comprises a distal binding group and the method comprises reacting the second chemical building block with the distal binding group of the first chemical building block to covalently attach the second chemical building block to the first chemical building block.

27. A method according to claim 26 wherein the second chemical building block comprises a proximal binding group and the method comprises reacting the proximal binding group of the second chemical building block with the distal binding group of the first chemical building block to covalently attach the second chemical building block to the first chemical building block.

28. A method according to claim 26 or claim 27 wherein the distal binding group of the first chemical building block is protected and the method comprises deprotecting the distal binding group of the first chemical building block before attachment of the second chemical building block.

29. A method according to any one of claims 26 to 28 wherein the method comprises capping unreacted distal binding groups of the first chemical building block that are not covalently attached to the second chemical building block.

30. A method according to any one of claims 25 to 29 comprising attaching a further chemical building block to the chemical building block at the end position of the chemical portion.

31. A method according to claim 30 wherein the chemical building block at the end position comprises a distal binding group and the method comprises reacting the further chemical building block with the distal binding group of the chemical building block at the end position to covalently attach the further chemical building block to form a chain of chemical building blocks with the further chemical building block at the end position.

32. A method according to claim 31 wherein the further chemical building block comprises a proximal binding group and the method comprises reacting the proximal binding group of the further chemical building block with the distal binding group of the chemical building block at the end position to covalently attach the further chemical building block.

33. A method according to claim 31 or claim 32 wherein the distal binding group of the chemical building block at the end position is protected and the method comprises deprotecting the distal binding group of the chemical building block at the end position before attachment of the further chemical building block.

34. A method according to any one of claims 31 to 33 wherein the method further comprises capping unreacted distal binding groups that are not covalently attached to the further chemical building block.

35. A method comprising repeating the steps of claims 30 to 34 one or more times.

36. A method according to any one of claims 1 to 35 wherein coding oligonucleotides encoding each chemical building block are added sequentially to the nascent member to form the coding nucleic acid portion, wherein the coding oligonucleotide encoding a chemical building block is added to the nascent member before, after or simultaneously with the addition of the chemical building block to the nascent binding member.

37. A method according to claim 36 wherein the method comprises covalently attaching a first coding oligonucleotide encoding the first chemical building block to the scaffold.

38. A method according to claim 37 wherein the scaffold comprises an attachment oligonucleotide and the method comprises covalently attaching the first coding oligonucleotide to the attachment oligonucleotide to form a coding nucleic acid portion attached to the scaffold.

39. A method according to claims 37 to 38 comprising covalently attaching a second coding oligonucleotide encoding the second chemical building block to the coding nucleic acid portion.

40. A method according to claim 39 comprising attaching one or more further coding oligonucleotides encoding further chemical building blocks to the coding nucleic acid portion.

41. A method according to any one of claims 1 to 40 comprising cross-linking two or more chemical building blocks in the chemical portion.

42. A method of producing a DNA encoded library comprising for each member the steps of;
providing a nascent member that comprises a scaffold connected to an anchor by a linker, wherein the anchor is connect to a solid support,
covalently attaching one or more chemical building blocks to the nascent member to form a chemical portion attached to the scaffold,
covalently attaching one or more coding oligonucleotides to the nascent member to form a coding nucleic acid portion attached to the anchor,
attaching a cleaving group to the chemical portion,
isolating the nascent member in a compartment, and
reacting the linker and the cleaving group, such that the linker is cleaved and the scaffold released from the solid support in the compartment.

43. A method of producing a DNA encoded library comprising for each member the steps of;
providing a nascent member comprising a scaffold and an anchor, wherein the scaffold is connected to a first linker by the anchor; and the anchor comprises an attachment oligonucleotide and is attached to a solid support,
covalently attaching one or more chemical building blocks to the nascent member to form a chemical portion attached to the scaffold,
covalently attaching one or more coding oligonucleotides to the attachment oligonucleotide to form a nucleic acid portion attached to the anchor,
attaching a second linker to the chemical portion to connect the chemical portion to the solid support,
cleaving the first linker,
isolating the nascent member in a compartment, and
cleaving the second linker, such that the second linker is cleaved and the chemical portion attached to the scaffold is released from the solid support in the compartment.

44. A method according to claim 42 or 43 wherein the anchor comprises an attachment oligonucleotide and the coding oligonucleotides encoding each chemical building block are added sequentially to the attachment oligonucleotide to form the coding nucleic acid portion attached to the anchor.

45. A method of producing a nucleic acid encoded library comprising for each member the steps of;
providing a nascent member comprising a scaffold connected to a solid support by a linker,
covalently attaching one or more chemical building blocks to the nascent member to form a chemical portion attached to the scaffold,
covalently attaching one or more coding oligonucleotides to the solid support to form a coding nucleic acid portion attached to the solid support,
attaching a cleaving group to the chemical portion,
isolating the nascent member in a compartment, and
reacting the linker and the cleaving group, such that the linker is cleaved, and the scaffold released from the solid support in the compartment.

46. A method of producing a nucleic acid encoded library comprising for each member the steps of;
providing a nascent member comprising a scaffold connected to a solid support by a first linker; and the solid support comprises an attachment oligonucleotide,
covalently attaching one or more chemical building blocks to the nascent member to form a chemical portion attached to the scaffold,
covalently attaching one or more coding oligonucleotides to the attachment oligonucleotide to form a coding nucleic acid portion attached to the solid support,
attaching a second linker to the chemical portion to connect the chemical portion to the solid support,
cleaving the first linker,
isolating the nascent member in a compartment, and
cleaving the second linker, such that the second linker is cleaved, and the chemical portion attached to the scaffold is released from the solid support in the compartment.

47. A method according to claim 45 or 46 wherein the solid support comprises an attachment oligonucleotide and the coding oligonucleotides encoding each chemical building block are added sequentially to the attachment oligonucleotide to form the coding nucleic acid portion.

48. A method according to any one of claims 42 to 47 wherein the compartment is a microvolume located at an addressable feature on an array.

49. A method according to any one of claims 1-48 comprising further purifying the compound or member or library of compounds or members.

50. A method according to any one of claims 1 to 49 further comprising producing a diverse population of compounds or library members, screening the diverse population for binding to a target molecule and identifying a library member in the population that binds to the target molecule.

51. A method according to claim 50 comprising;
providing nascent member comprising a labelled scaffold connected to a solid support by a linker,
covalently attaching the chemical portion of an identified library member to the scaffold to produce a labelled nascent member that comprises said chemical portion,
attaching a cleaving group to the chemical portion or scaffold,
reacting the linker with the cleaving group, thereby cleaving the linker and releasing the member from the solid support, and
determining the binding of the released labelled member to the target molecule.

52. A method according to claim 50 comprising;
providing nascent member comprising a labelled scaffold connected to a solid support by a first linker,
covalently attaching the chemical portion of an identified library member to the scaffold to produce a labelled nascent member that comprises said chemical portion,
connecting the chemical portion to the solid support with a second linker,
cleaving the first linker,
cleaving the second linker and releasing the member from the solid support, and
determining the binding of the released labelled member to the target molecule.

53. A nucleic acid encoded library produced by a method according to any one of claims 1 to 52.
